SEQUENCE LISTING (77 sequences)

SEQ ID NO: 1 - DNA, 24 bp, synthetic construct
  misc_feature 1..24: note = Synthetic: Chr.8 control
  source 1..24: mol_type = other DNA
caccgacatttctttccccactgg

SEQ ID NO: 2 - DNA, 24 bp, synthetic construct
  misc_feature 1..24: note = Synthetic: Chr.8 control
  source 1..24: mol_type = other DNA
aaacccagtggggaaagaaatgtc

SEQ ID NO: 3 - DNA, 25 bp, synthetic construct
  misc_feature 1..25: note = Synthetic: Acly sg1
  source 1..25: mol_type = other DNA
caccggggcgtacttgaaccggttc

SEQ ID NO: 4 - DNA, 25 bp, synthetic construct
  misc_feature 1..25: note = Synthetic: Acly sg1
  source 1..25: mol_type = other DNA
aaacgaaccggttcaagtacgcccc

SEQ ID NO: 5 - DNA, 25 bp, synthetic construct
  misc_feature 1..25: note = Synthetic: Acly sg2
  source 1..25: mol_type = other DNA
caccggaaccggttcaagtacgccc

SEQ ID NO: 6 - DNA, 25 bp, synthetic construct
  misc_feature 1..25: note = Synthetic: Acly sg2
  source 1..25: mol_type = other DNA
aaacgggcgtacttgaaccggttcc

SEQ ID NO: 7 - DNA, 25 bp, synthetic construct
  misc_feature 1..25: note = Synthetic: Aco2 sg1
  source 1..25: mol_type = other DNA
caccggccaaccaggagatcgagcg

SEQ ID NO: 8 - DNA, 25 bp, synthetic construct
  misc_feature 1..25: note = Synthetic: Aco2 sg1
  source 1..25: mol_type = other DNA
aaaccgctcgatctcctggttggcc

SEQ ID NO: 9 - DNA, 25 bp, synthetic construct
  misc_feature 1..25: note = Synthetic: Slc25a1 sg1
  source 1..25: mol_type = other DNA
caccgcttcacgtattcggtcggga

SEQ ID NO: 10 - DNA, 25 bp, synthetic construct
  misc_feature 1..25: note = Synthetic: Slc25a1 sg1
  source 1..25: mol_type = other DNA
aaactcccgaccgaatacgtgaagc

SEQ ID NO: 11 - DNA, 25 bp, synthetic construct
  misc_feature 1..25: note = Synthetic: Slc25a1 sg2
  source 1..25: mol_type = other DNA
caccggagaggactattgtgcggtc

SEQ ID NO: 12 - DNA, 25 bp, synthetic construct
  misc_feature 1..25: note = Synthetic: Slc25a1 sg2
  source 1..25: mol_type = other DNA
aaacgaccgcacaatagtcctctcc

SEQ ID NO: 13 - DNA, 25 bp, synthetic construct
  misc_feature 1..25: note = Synthetic: Mdh1 sg1
  source 1..25: mol_type = other DNA
caccgttggacatcacccccatgat

SEQ ID NO: 14 - DNA, 25 bp, synthetic construct
  misc_feature 1..25: note = Synthetic: Mdh1 sg1
  source 1..25: mol_type = other DNA
aaacatcatgggggtgatgtccaac

SEQ ID NO: 15 - DNA, 25 bp, synthetic construct
  misc_feature 1..25: note = Synthetic: Mdh1 sg2
  source 1..25: mol_type = other DNA
caccgtcagccagctgtcgtctttc

SEQ ID NO: 16 - DNA, 25 bp, synthetic construct
  misc_feature 1..25: note = Synthetic: Mdh1 sg2
  source 1..25: mol_type = other DNA
aaacgaaagacgacagctggctgac

SEQ ID NO: 17 - DNA, 25 bp, synthetic construct
  misc_feature 1..25: note = Synthetic: ACACA sg1
  source 1..25: mol_type = other DNA
caccgaagtgtatctgagctgacgg

SEQ ID NO: 18 - DNA, 25 bp, synthetic construct
  misc_feature 1..25: note = Synthetic: ACACA sg1
  source 1..25: mol_type = other DNA
aaacccgtcagctcagatacacttc

SEQ ID NO: 19 - DNA, 25 bp, synthetic construct
  misc_feature 1..25: note = Synthetic: ACACA sg2
  source 1..25: mol_type = other DNA
caccgctcacaacgagatttcactg

SEQ ID NO: 20 - DNA, 25 bp, synthetic construct
  misc_feature 1..25: note = Synthetic: ACACA sg2
  source 1..25: mol_type = other DNA
aaaccagtgaaatctcgttgtgagc

SEQ ID NO: 21 - DNA, 25 bp, synthetic construct
  misc_feature 1..25: note = Synthetic: sgTcf7l1
  source 1..25: mol_type = other DNA
caccgccgggcaagctcatagtatt

SEQ ID NO: 22 - DNA, 25 bp, synthetic construct
  misc_feature 1..25: note = Synthetic: sgTcf7l1
  source 1..25: mol_type = other DNA
aaacaatactatgagcttgcccggc

SEQ ID NO: 23 - DNA, 97 bp, synthetic construct
  misc_feature 1..97: note = Synthetic: shRen
  source 1..97: mol_type = other DNA
tgctgttgacagtgagcgcaggaattataatgcttatctatagtgaagccacagatgtatagataagcattataattcctatgcctactgcctcgga

SEQ ID NO: 24 - DNA, 97 bp, synthetic construct
  misc_feature 1..97: note = Synthetic: shAcly-1
  source 1..97: mol_type = other DNA
tgctgttgacagtgagcgcctgtattaatctgatttttaatagtgaagccacagatgtattaaaaatcagattaatacagttgcctactgcctcgga

SEQ ID NO: 25 - DNA, 97 bp, synthetic construct
  misc_feature 1..97: note = Synthetic: shAcly-2
  source 1..97: mol_type = other DNA
tgctgttgacagtgagcgctgtaacatacaagtgtttaaatagtgaagccacagatgtatttaaacacttgtatgttacaatgcctactgcctcgga

SEQ ID NO: 26 - DNA, 97 bp, synthetic construct
  misc_feature 1..97: note = Synthetic: shAco2-1
  source 1..97: mol_type = other DNA
tgctgttgacagtgagcgctcaccagatcattctagaaaatagtgaagccacagatgtattttctagaatgatctggtgaatgcctactgcctcgga

SEQ ID NO: 27 - DNA, 97 bp, synthetic construct
  misc_feature 1..97: note = Synthetic: shAco2-2
  source 1..97: mol_type = other DNA
tgctgttgacagtgagcgacagtatgaccaagtgattgaatagtgaagccacagatgtattcaatcacttggtcatactggtgcctactgcctcgga

SEQ ID NO: 28 - DNA, 20 bp, synthetic construct
  misc_feature 1..20: note = Synthetic: Actin F
  source 1..20: mol_type = other DNA
gctcttttccagccttcctt

SEQ ID NO: 29 - DNA, 20 bp, synthetic construct
  misc_feature 1..20: note = Synthetic: Actin R
  source 1..20: mol_type = other DNA
cttctgcatcctgtcagcaa

SEQ ID NO: 30 - DNA, 20 bp, synthetic construct
  misc_feature 1..20: note = Synthetic: Nanog F
  source 1..20: mol_type = other DNA
aagatgcggactgtgttctc

SEQ ID NO: 31 - DNA, 20 bp, synthetic construct
  misc_feature 1..20: note = Synthetic: Nanog R
  source 1..20: mol_type = other DNA
cgcttgcacttcatcctttg

SEQ ID NO: 32 - DNA, 20 bp, synthetic construct
  misc_feature 1..20: note = Synthetic: Esrrb F
  source 1..20: mol_type = other DNA
aacagcccctacctgaacct

SEQ ID NO: 33 - DNA, 20 bp, synthetic construct
  misc_feature 1..20: note = Synthetic: Esrrb R
  source 1..20: mol_type = other DNA
tgccaattcacagagagtgg

SEQ ID NO: 34 - DNA, 20 bp, synthetic construct
  misc_feature 1..20: note = Synthetic: Klf2 F
  source 1..20: mol_type = other DNA
taaaggcgcatctgcgtaca

SEQ ID NO: 35 - DNA, 20 bp, synthetic construct
  misc_feature 1..20: note = Synthetic: Klf2 R
  source 1..20: mol_type = other DNA
cgcacaagtggcactgaaag

SEQ ID NO: 36 - DNA, 21 bp, synthetic construct
  misc_feature 1..21: note = Synthetic: Rex1 F
  source 1..21: mol_type = other DNA
tccatggcatagttccaacag

SEQ ID NO: 37 - DNA, 22 bp, synthetic construct
  misc_feature 1..22: note = Synthetic: Rex1 R
  source 1..22: mol_type = other DNA
taactgattttctgccgtatgc

SEQ ID NO: 38 - DNA, 19 bp, synthetic construct
  misc_feature 1..19: note = Synthetic: Klf4 F
  source 1..19: mol_type = other DNA
cgggaagggagaagacact

SEQ ID NO: 39 - DNA, 18 bp, synthetic construct
  misc_feature 1..18: note = Synthetic: Klf4 R
  source 1..18: mol_type = other DNA
gagttcctcacgccaacg

SEQ ID NO: 40 - DNA, 20 bp, synthetic construct
  misc_feature 1..20: note = Synthetic: Sox1 F
  source 1..20: mol_type = other DNA
cctcggatctctggtcaagt

SEQ ID NO: 41 - DNA, 22 bp, synthetic construct
  misc_feature 1..22: note = Synthetic: Sox1 R
  source 1..22: mol_type = other DNA
gcaggtacatgctgatcatctc

SEQ ID NO: 42 - DNA, 19 bp, synthetic construct
  misc_feature 1..19: note = Synthetic: Otx2 F
  source 1..19: mol_type = other DNA
gacccggtacccagacatc

SEQ ID NO: 43 - DNA, 24 bp, synthetic construct
  misc_feature 1..24: note = Synthetic: Otx2 R
  source 1..24: mol_type = other DNA
gctcttcgattcttaaaccatacc

SEQ ID NO: 44 - DNA, 22 bp, synthetic construct
  misc_feature 1..22: note = Synthetic: Fgf5 F
  source 1..22: mol_type = other DNA
aaactccatgcaagtgccaaat

SEQ ID NO: 45 - DNA, 22 bp, synthetic construct
  misc_feature 1..22: note = Synthetic: Fgf5 R
  source 1..22: mol_type = other DNA
tctcggcctgtcttttcagttc

SEQ ID NO: 46 - DNA, 20 bp, synthetic construct
  misc_feature 1..20: note = Synthetic: Oct4 F
  source 1..20: mol_type = other DNA
tggatcctcgaacctggcta

SEQ ID NO: 47 - DNA, 20 bp, synthetic construct
  misc_feature 1..20: note = Synthetic: Oct4 R
  source 1..20: mol_type = other DNA
ccctccgcagaactcgtatg

SEQ ID NO: 48 - DNA, 25 bp, synthetic construct
  misc_feature 1..25: note = Synthetic: Acly sg1 + sg2 F
  source 1..25: mol_type = other DNA
accggcaaagaactcctgtacaagt

SEQ ID NO: 49 - DNA, 22 bp, synthetic construct
  misc_feature 1..22: note = Synthetic: Acly sg1 + sg2 R
  source 1..22: mol_type = other DNA
caagtctgcaatcacggcgctc

SEQ ID NO: 50 - DNA, 23 bp, synthetic construct
  misc_feature 1..23: note = Synthetic: Aco2 sg1 F
  source 1..23: mol_type = other DNA
tgcacacagatgcttctgtgcct

SEQ ID NO: 51 - DNA, 22 bp, synthetic construct
  misc_feature 1..22: note = Synthetic: Aco2 sg1 R
  source 1..22: mol_type = other DNA
atagccatctgggctgtggcat

SEQ ID NO: 52 - DNA, 20 bp, synthetic construct
  misc_feature 1..20: note = Synthetic: Slc25a1 sg1 F
  source 1..20: mol_type = other DNA
tgtccctagccccaggcatt

SEQ ID NO: 53 - DNA, 20 bp, synthetic construct
  misc_feature 1..20: note = Synthetic: Slc25a1 sg1 R
  source 1..20: mol_type = other DNA
ttgccgcacgcagtctccta

SEQ ID NO: 54 - DNA, 23 bp, synthetic construct
  misc_feature 1..23: note = Synthetic: Slc25a1 sg2 F
  source 1..23: mol_type = other DNA
ttgggatgttcgagttcctcagc

SEQ ID NO: 55 - DNA, 22 bp, synthetic construct
  misc_feature 1..22: note = Synthetic: Slc25a1 sg2 R
  source 1..22: mol_type = other DNA
ttcccaccttgacagcctccat

SEQ ID NO: 56 - DNA, 24 bp, synthetic construct
  misc_feature 1..24: note = Synthetic: Mdh1 sg1 F
  source 1..24: mol_type = other DNA
gcagctactaagcaagctatggca

SEQ ID NO: 57 - DNA, 22 bp, synthetic construct
  misc_feature 1..22: note = Synthetic: Mdh1 sg1 R
  source 1..22: mol_type = other DNA
gacttcccaactcaccctgcag

SEQ ID NO: 58 - DNA, 24 bp, synthetic construct
  misc_feature 1..24: note = Synthetic: Mdh1 sg2 F
  source 1..24: mol_type = other DNA
ccagatgtcaatcatgccaaggtg

SEQ ID NO: 59 - DNA, 26 bp, synthetic construct
  misc_feature 1..26: note = Synthetic: Mdh1 sg2 R
  source 1..26: mol_type = other DNA
gtgtcatagatcaggtcacatcagac

SEQ ID NO: 60 - DNA, 23 bp, synthetic construct
  misc_feature 1..23: note = Synthetic: ACACA sg1 F
  source 1..23: mol_type = other DNA
cctggtgaagctggacctagaag

SEQ ID NO: 61 - DNA, 22 bp, synthetic construct
  misc_feature 1..22: note = Synthetic: ACACA sg1 R
  source 1..22: mol_type = other DNA
tcatagaagatgggccagggaa

SEQ ID NO: 62 - DNA, 21 bp, synthetic construct
  misc_feature 1..21: note = Synthetic: ACACA sg2 F
  source 1..21: mol_type = other DNA
gcatgtctggcttgcacctag

SEQ ID NO: 63 - DNA, 25 bp, synthetic construct
  misc_feature 1..25: note = Synthetic: ACACA sg2 R
  source 1..25: mol_type = other DNA
ggagacagaaaggatagccctgcat

SEQ ID NO: 64 - DNA, 243 bp, synthetic construct
  misc_feature 1..243: note = Synthetic: ACO2-1 allele 1
  source 1..243: mol_type = other DNA
tgcacacagatgcttctgtgcctgctcactgccacacatgtgagagggaaaagctgtgccaggctgagtcccaaagctacttccttctttgtgcccgtaggttgaaccggcctcttactctctcagagaagattgtatatggacacctggatgacccagccaaccaggagatcggggaaagacatacctgcgtttacggcccgaccgggtggccatgcaggatgccacagcccagatggctat

SEQ ID NO: 65 - DNA, 244 bp, synthetic construct
  misc_feature 1..244: note = Synthetic: ACO2-1 allele 2
  source 1..244: mol_type = other DNA
tgcacacagatgcttctgtgcctgctcactgccacacatgtgagagggaaaagctgtgccaggctgagtcccaaagctacttccttctttgtgcccgtaggttgaaccggcctcttactctctcagagaagattgtatatggacacctggatgacccagccaaccaggagatcaggggaaagacatacctgcgtttacggcccgaccgggtggccatgcaggatgccacagcccagatggctat

SEQ ID NO: 66 - DNA, 236 bp, synthetic construct
  misc_feature 1..236: note = Synthetic: ACO2-2 allele 1
  source 1..236: mol_type = other DNA
tgcacacagatgcttctgtgcctgctcactgccacacatgtgagagggaaaagctgtgccaggctgagtcccaaagctacttccttctttgtgcccgtaggttgaaccggcctcttactctctcagagaagattgtatatggacacctggatgacccagccaaccaggagaaagacatacctgcgtttacggcccgaccgggtggccatgcaggatgccacagcccagatggctat

SEQ ID NO: 67 - DNA, 271 bp, synthetic construct
  misc_feature 1..271: note = Synthetic: ACO2-2 allele 2
  source 1..271: mol_type = other DNA
tgcacacagatgcttctgtgcctgctcactgccacacatgtgagagggaaaagctgtgccaggctgagtcccaaagctacttccttctttgtgcccgtaggttgaaccggcctcttactctctcagagaagattgtatatggacacctggatgacccagccaaccaggagatcgagctgctggggaaagacatcgagcagcggggaaagacatacctgcgtttacggcccgaccgggtggccatgcaggatgccacagcccagatggctat

SEQ ID NO: 68 - DNA, 280 bp, synthetic construct
  misc_feature 1..280: note = Synthetic: MDH1-1 allele 1
  source 1..280: mol_type = other DNA
gcagctactaagcaagctatggcatctctcttccataattatgactaattagtgggaactaaatatatacaaacaaaaatagggtgggactttgtgtgagagagaatgcagtaaccaccccgaccttcaccataaccgcattgcctgctgtccttgctctttggcagcccatcattcttgtgctgttggacatcacccccattgatgggtgttctggacggtgtcctgatggaactgcaagactgtgcccttccccttctgcagggtgagttgggaagtc

SEQ ID NO: 69 - DNA, 276 bp, synthetic construct
  misc_feature 1..276: note = Synthetic: MDH1-2 allele 1
  source 1..276: mol_type = other DNA
ccagatgtcaatcatgccaaggtgaaactgcaaggaaaggaagtcggtgtgtatgaagccctgaagacgacagctggctgaagggagagttcatcacggtaagaaggatgtgaaccctctgagcagccagaggagacacacagcacactgctgagctgagtgtctgccagggccctgtgggaccctcaaggacagctgctgttttactctaatatattcttttgaagtgtgcaaaaagatggttttcattgtctgatgtgacctgatctatgacac

SEQ ID NO: 70 - DNA, 229 bp, synthetic construct
  misc_feature 1..229: note = Synthetic: MDH1-2 allele 2
  source 1..229: mol_type = other DNA
ccagatgtcaatcatgccaaggtgagctggctgaagggagagttcatcacggtaagaaggatgtgaaccctctgagcagccagaggagacacacagcacactgctgagctgagtgtctgccagggccctgtgggaccctcaaggacagctgctgttttactctaatatattcttttgaagtgtgcaaaaagatggttttcattgtctgatgtgacctgatctatgacac

SEQ ID NO: 71 - DNA, 229 bp, synthetic construct
  misc_feature 1..229: note = Synthetic: ACLY-1 allele 1
  source 1..229: mol_type = other DNA
caagtctgcaatcacggcgctccttgagacacctccaacaaagcaaatgctttcccctcctcagagcctgagctggacagaagggctcagggtgcaggcagagcttacctggctgagcagccacgggtggtcctgcagcagatgggcccagtctgtgtcgggagtcacccgggcgtacttggatggccgaggtggtgcagatgtacttgtacaggagttctttgccggt

SEQ ID NO: 72 - DNA, 236 bp, synthetic construct
  misc_feature 1..236: note = Synthetic: ACLY-1 allele 2
  source 1..236: mol_type = other DNA
caagtctgcaatcacggcgctccttgagacacctccaacaaagcaaatgctttcccctcctcagagcctgagctggacagaagggctcagggtgcaggcagagcttacctggctgagcagccacgggtggtcctgcagcagatgggcccagtctgtgtcgggagtcacccgggcgtacttgaaccggggatggccgaggtggtgcagatgtacttgtacaggagttctttgccggt

SEQ ID NO: 73 - DNA, 231 bp, synthetic construct
  misc_feature 1..231: note = Synthetic: ACLY-2 allele 1
  source 1..231: mol_type = other DNA
caagtctgcaatcacggcgctccttgagacacctccaacaaagcaaatgctttcccctcctcagagcctgagctggacagaagggctcagggtgcaggcagagcttacctggctgagcagccacgggtggtcctgcagcagatgggcccagtctgtgtcgggagtctacatctgcaccacctcggccatccagaaccggttcaagtacttgtacaggagttctttgccggt

SEQ ID NO: 74 - DNA, 231 bp, synthetic construct
  misc_feature 1..231: note = Synthetic: SLC25A1-1 allele 1
  source 1..231: mol_type = other DNA
tgtccctagccccaggcatttggtccccacaggcggcctggctggaggcatcgaaatctgcatcaccttcccgaatacgtgaagacgcagctgcagctagatgaacgagcgaacccaccgcggtaccggggcatcggtaaggagggactcccgggggtggctagggggacactgcggagcagaggagctagggtcttgaccgcgcctcccgtaggagactgcgtgcggcaa

SEQ ID NO: 75 - DNA, 233 bp, synthetic construct
  misc_feature 1..233: note = Synthetic: SLC25A1-1 allele 2
  source 1..233: mol_type = other DNA
tgtccctagccccaggcatttggtccccacaggcggcctggctggaggcatcgaaatctgcatcaccttcgaccgaatacgtgaagacgcagctgcagctagatgaacgagcgaacccaccgcggtaccggggcatcggtaaggagggactcccgggggtggctagggggacactgcggagcagaggagctagggtcttgaccgcgcctcccgtaggagactgcgtgcggcaa

SEQ ID NO: 76 - DNA, 219 bp, synthetic construct
  misc_feature 1..219: note = Synthetic: SLC25A1-2 allele 1
  source 1..219: mol_type = other DNA
ttgggatgttcgagttcctcagcaaccacatgcgggatgcccaaggtaggctcgacagcaggagaggactattgtgccggcgtggcagaagcagtggtagtcgtgtgccctatggagaccatcaaggtgagggattgagcctttcaggggtagccagggtatcttcgggctcccaggttttaatggcatgtgcccccatggaggctgtcaaggtgggaa

SEQ ID NO: 77 - DNA, 224 bp, synthetic construct
  misc_feature 1..224: note = Synthetic: SLC25A1-2 allele 2
  source 1..224: mol_type = other DNA
ttgggatgttcgagttcctcagcaaccacatgcgggatgcccaaggtaggctcgacagcaggagaggacattgtctgggtgccggcgtggcagaagcagtggtagtcgtgtgccctatggagaccatcaaggtgagggattgagcctttcaggggtagccagggtatcttcgggctcccaggttttaatggcatgtgcccccatggaggctgtcaaggtgggaa

(19) United States
(12) Patent Application Publication
(10) Pub. No.: US 2024/0392239 A1
(43) Pub. Date: Nov. 28, 2024

(54) METHODS FOR MODULATING CELL PLURIPOTENCY AND SELF-RENEWAL PROPERTY

(71) Applicant: Memorial Sloan-Kettering Cancer Center, New York, NY (US)

(72) Inventors: Lydia W.S. Finley, New York, NY (US); Paige K, Arnold, New York, NY (US); Benjamin T. Jackson, New York, NY (US)

(73) Assignee: Memorial Sloan-Kettering Cancer Center, New York, NY (US)

(21) Appl. No.: 18/647,236

(22) Filed: Apr. 26, 2024

Related U.S. Application Data
(63) Continuation of application No. PCT/US2022/048196, filed on Oct. 28, 2022.
(60) Provisional application No. 63/272,940, filed on Oct. 28, 2021.

(51) Int. Cl. C12N 5/0735

(52) U.S. Cl. CPC C12N 5/0606; C12N 2501/235; C12N 2501/72; C12N 2510/00

(57) ABSTRACT

The present disclosure relates to methods for modulating (e.g., maintaining) pluripotency and self-renewal property of cells (e.g., stem cells) by blocking the non-canonical tricarboxylic acid (TCA) cycle (e.g. using an inhibitor of ATP citrate lyase (ACL) or acetate), and kits and compositions relating thereto.

### CROSS-REFERENCE TO RELATED APPLICATIONS

This application is a continuation of International Patent Application No. PCT/US2022/048196, filed Oct. 28, 2022, which claims priority to U.S. Provisional Patent Application No. 63/272,940, filed on Oct. 28, 2021, the content of which is incorporated by reference in its entirety, and to which priority is claimed.

### 1. GRANT INFORMATION

This invention was made with government support under HD098824, and CA252305awarded by National Institutes of Health. The government has certain rights in the invention.

### SEQUENCE LISTING

The present application contains a Sequence Listing which has been submitted via EFS-Web and is hereby incorporated by reference in its entirety. Said Sequence Listing, created on Apr. 26, 2024, is named 0727341624.xml and is 102203 bytes in size.

### 2. INTRODUCTION

The present disclosure relates to methods for modulating (e.g., maintaining) pluripotency and self-renewal property of cells (e.g., stem cells) by blocking the non-canonical tricarboxylic acid (TCA) cycle (e.g., using an inhibitor of ATP citrate lyase (ACL) or acetate), and kits and compositions relating thereto.

### 3. BACKGROUND

Mammalian cells exploit diverse strategies to meet their metabolic demands. In particular, cells exhibit heterogeneity in the choice of nutrients to fuel the TCA cycle and the activity of many enzymatic reactions in the TCA cycle¹⁻⁴. While the understanding of why TCA cycle activity varies between different cells is incomplete, emerging evidence indicates that metabolic phenotypes are determined by a combination of cell genotype, phenotype, lineage and environmental milicu²⁻⁴. For example, while cancer cells growing in vitro rely on glutamine as a major source of TCA cycle intermediates, glutamine is a relatively minor source of TCA cycle carbon for cancer cells growing in vivo⁵⁻⁷. Likewise, stem cells alter TCA cycle substrate preferences as they undergo lineage commitment and terminal differentiation⁸⁻¹⁰. These observations suggest that cells can selectively engage components of the TCA cycle to meet their metabolic demands.

It is still unknown whether enzymes involved in the TCA cycle might form discrete functional modules beyond the canonical pathway. The variable essentiality of TCA cycle enzymes across hundreds of cultured cancer cell lines provides a unique opportunity to uncover potential metabolic networks that may underlie cell-state specific metabolic diversity, as genes participating in the same metabolic pathway exhibit similar patterns of essentiality¹¹⁻¹³. Studies correlating gene essentiality profiles have been used to delineate functional gene networks, enabling identification of previously unknown members of specific metabolic pathways and assigning function to unidentified genes^{12.14}. While the enzymes that comprise the TCA cycle are well defined, how these enzymes assemble into functional networks remains largely unexplored.

### 4. SUMMARY OF THE INVENTION

The present disclosure provides methods for modulating (e.g., maintaining) pluripotency and self-renewal property of cells (e.g., stem cells) by blocking the non-canonical TCA cycle (e.g., using an inhibitor of ATP citrate lyase (ACL) or acetate). It is based on the discovery that blocking the non-canonical TCA cycle prevented stem cells from exiting the pluripotent state.

In certain embodiments, the present disclosure provides a method for maintaining pluripotency of cells, comprising blocking non-canonical tricarboxylic acid (TCA) cycle of the cells. In certain embodiments, the present disclosure provides a method for maintaining self-renewal property of the cells, comprising blocking non-canonical tricarboxylic acid (TCA) cycle of the cells.

In certain embodiments, blocking the non-canonical TCA cycle comprises contacting the cells with an ATP citrate lyase (ACL) inhibitor. In certain embodiments, the ACL inhibitor is a synthetic ACL inhibitor, a natural ACL inhibitor, or a combination thereof.

In certain embodiments, the synthetic ACL inhibitor is selected from the group consisting of BMS-303141, sulfoximine, 2,2-difluorocitrate, expoxide, SB-201076, SB-204990, medica 16, 3-thiadicarboxylic acid, ETC-1002, ETC-1002-CoA, 2-hydroxy-narylbenzenesulfonamide, furan carboxylate derivatives, derivatives thereof, and combinations thereof. In certain embodiments, the synthetic ACL inhibitor is BMS-303141.

In certain embodiments, the natural ACL inhibitor is selected from the group consisting of (−)-hydroxycitric acid, 2-chloro-1,3,8-trihydroxy-6-methylanthracen-9(10H)-one, antimycins A2, antimycins A8, purpurone, radicicol, cucurbitacin B, derivatives thereof, and combinations thereof.

In certain embodiments, the concentration of the ACL inhibitor is between about 10 μM and about 100 μM. In certain embodiments, the concentration of the ACL inhibitor is about 50 μM.

In certain embodiments, the cells are contacted with the ACL inhibitor for at least about 12 hours. In certain embodiments, the cells are contacted with the ACL inhibitor for about 24 hours.

In certain embodiments, blocking the non-canonical TCA cycle comprises contacting the cells with acetate.

In certain embodiments, the cells are pluripotent cells, stem cells, progenitor cells, or a combination thereof. In certain embodiments, the stem cells are selected from the group consisting of pluripotent stem cells, embryonic stem cells (ESCs), induced pluripotent stem cells (iPSCs), parthenogenetic stem cells, primordial germ cell-like pluripotent stem cells, epiblast stem cells, F-class pluripotent stem cells, somatic stem cells, cancer stem cells, any other cell capable of lineage specific differentiation, and combinations thereof.

In certain embodiments, the cells are ESCs. In certain embodiments, the ESCs are human ESCs or mouse ESCs.

In certain embodiments, the present disclosure provides plurality of cells, wherein pluripotency of the cells is maintained, after blocking the non-canonical tricarboxylic acid (TCA) cycle of the cells.

In certain embodiments, the present disclosure provides plurality of cells, wherein self-renewal property of the cells is maintained, after blocking the non-canonical tricarboxylic acid (TCA) cycle of the cells.

In certain embodiments, blocking the non-canonical TCA cycle comprises contacting the cells with an ATP citrate lyase (ACL) inhibitor. In certain embodiments, the ACL inhibitor is a synthetic ACL inhibitor, a natural ACL inhibitor, or a combination thereof.

In certain embodiments, the synthetic ACL inhibitor is selected from the group consisting of BMS-303141, sulfoximine, 2,2-difluorocitrate, expoxide, SB-201076, SB-204990, medica 16, 3-thiadicarboxylic acid, ETC-1002, ETC-1002-CoA, 2-hydroxy-narylbenzenesulfonamide, furan carboxylate derivatives, derivatives thereof, and combinations thereof. In certain embodiments, the synthetic ACL inhibitor is BMS-303141.

In certain embodiments, the natural ACL inhibitor is selected from the group consisting of (−)-hydroxycitric acid, 2-chloro-1,3,8-trihydroxy-6-methylanthracen-9(10H)-one, antimycins A2, antimycins A8, purpurone, radicicol, cucurbitacin B, derivatives thereof, and combinations thereof.

In certain embodiments, the concentration of the ACL inhibitor is between about 10 μM and about 100 μM. In certain embodiments, the concentration of the ACL inhibitor is about 50 μM.

In certain embodiments, the cells are contacted with the ACL inhibitor for at least about 12 hours. In certain embodiments, the cells are contacted with the ACL inhibitor for about 24 hours.

In certain embodiments, blocking the non-canonical TCA cycle comprises contacting the cells with acetate.

In certain embodiments, the cells are pluripotent cells, stem cells, progenitor cells, or a combination thereof. In certain embodiments, the stem cells are selected from the group consisting of pluripotent stem cells, embryonic stem cells (ESCs), induced pluripotent stem cells (iPSCs), parthenogenetic stem cells, primordial germ cell-like pluripotent stem cells, epiblast stem cells, F-class pluripotent stem cells, somatic stem cells, cancer stem cells, any other cell capable of lineage specific differentiation, and combinations thereof.

In certain embodiments, the cells are ESCs. In certain embodiments, the ESCs are human ESCs or mouse ESCs.

In certain embodiments, the present disclosure provides a composition comprising the presently disclosed cells.

In certain embodiments, the present disclosure provides a kit for maintaining pluripotency or self-renewal property of cells, comprising: an agent that blocks non-canonical tricarboxylic acid (TCA) cycle, and a plurality of cells.

In certain embodiments, the cells are pluripotent cells, stem cells, progenitor cells, or a combination thereof. In certain embodiments, the stem cells are selected from the group consisting of pluripotent stem cells, embryonic stem cells (ESCs), induced pluripotent stem cells (iPSCs), parthenogenetic stem cells, primordial germ cell-like pluripotent stem cells, epiblast stem cells, F-class pluripotent stem cells, somatic stem cells, cancer stem cells, any other cell capable of lineage specific differentiation, and combinations thereof.

In certain embodiments, the agent is an ATP citrate lyase (ACL) inhibitor. In certain embodiments, the ACL inhibitor is a synthetic ACL inhibitor, a natural ACL inhibitor, or a combination thereof.

In certain embodiments, the synthetic ACL inhibitor is selected from the group consisting of BMS-303141, sulfoximine, 2,2-difluorocitrate, expoxide, SB-201076, SB-204990, medica 16, 3-thiadicarboxylic acid, ETC-1002, ETC-1002-CoA, 2-hydroxy-narylbenzenesulfonamide, furan carboxylate derivatives, derivatives thereof, and combinations thereof. In certain embodiments, the synthetic ACL inhibitor is BMS-303141.

In certain embodiments, the natural ACL inhibitor is selected from the group consisting of (−)-hydroxycitric acid, 2-chloro-1,3,8-trihydroxy-6-methylanthracen-9(10H)-one, antimycins A2, antimycins A8, purpurone, radicicol, cucurbitacin B, derivatives thereof, and combinations thereof.

In certain embodiments, the concentration of the ACL inhibitor is between about 10 μM and about 100 μM. In certain embodiments, the concentration of the ACL inhibitor is about 50 μM.

In certain embodiments, the cells are contacted with the ACL inhibitor for at least about 12 hours. In certain embodiments, the cells are contacted with the ACL inhibitor for about 24 hours.

In certain embodiments, the kit further comprises instructions of contacting the cells with the ACL inhibitor for at least about 12 hours.

In certain embodiments, the kit further comprises instructions of contacting the cells with the ACL inhibitor for about 24 hours.

In certain embodiments, the agent is acetate.

### 5. BRIEF DESCRIPTION OF THE DRAWINGS

FIGS. 1A-1E. Genetic coessentiality mapping of metabolic enzymes reveals two TCA cycle modules in cancer cell lines. FIG. 1A. Two-dimensional network diagram representing gene essentiality score correlations between genes from four core metabolic pathways (GO terms: tricarboxylic acid cycle, canonical glycolysis, 1-carbon metabolic process and fatty-acyl-CoA metabolic process). The strength of the correlation between genes is represented by both the length and thickness of the connecting edge. FIG. 1B. Schematic representing the two distinct TCA cycle modules that emerge from gene essentiality score correlation clustering. Left, TCA cycle genes are annotated on the traditional TCA cycle pathway. Genes in cluster 2 are shown in bold. Right, cluster 1 genes are annotated on a ‘non-canonical’ TCA cycle in which citrate is exported to the cytoplasm and cleaved by ACL to liberate acetyl-CoA and regenerate oxaloacetate, which can yield malate for mitochondrial import and oxaloacetate regeneration. Genes are colored according to their GO term (red, TCA cycle; blue, fatty acyl metabolism) or grey (no significant correlation). FIG. 1C. Schematic depicting possible fates for citrate containing 2 carbons derived from [U-¹³C] glucose. Top, m+2 labeled citrate will generate m+2 labeled malate downstream if metabolized by aconitase in the traditional TCA cycle. Bottom, m+2 labeled citrate cleaved in the cytoplasm by ACL will lose its two heavy-labeled carbons and any four-carbon metabolites generated from this backbone will be unlabeled. FIG. 1D. Fractional m+2 enrichment of citrate and downstream TCA cycle intermediates fumarate and malate in 82 non-small cell lung cancer (NSCLC) cell lines cultured in medium containing [U-¹³C] glucose for 6 h. Data mined from Chen et al., 2019. FIG. 1E. Fractional enrichment of glucose-derived malate m+2 relative to citrate m+2 (Mal+2/Cit+2) in three NSCLC cell lines following incubation with vehicle or 50 μM BMS-303141 (ACLi) for 24 h. Data are means±SD, n=3 independent replicates. Significance was assessed in comparison to citrate by one-way ANOVA (d) or vehicle-treated cells by two-way ANOVA (e) with Sidak's multiple comparisons post-test (***, P<0.001; ****, P<0.0001).

FIGS. 2A-2I: ACL loss disrupts TCA cycle metabolism in ESCs. FIG. 2A. Fractional m+2 enrichment of citrate and malate in mouse ESCs cultured in medium containing [U-¹³C] glucose. FIG. 2B. Fractional enrichment of malate m+2 relative to citrate m+2 (Mal+2/Cit+2) derived from [U-¹³C] glucose in ESCs following treatment with vehicle or 50 UM BMS-303141 (ACLi) for 24 h. FIGS. 2C and 2D. Fractional m+2 enrichment of citrate (Cit), fumarate (Fum), malate (Mal) and aspartate (Asp) (C) or Mal+2/Cit+2 ratio (D) in control (Ctrl) and Acly-edited (ACLY-1 and ACLY-2) mouse ESCs cultured in medium containing [U-¹³C] glucose. FIG. 2E. Steady-state levels of TCA cycle metabolites in Acly-edited mouse ESCs. Levels are represented as the fold change (expressed in log₂) relative to control cells. FIG. 2F. Schematic depicting deuterium label transfer from [4-²H] glucose first onto NADH during glycolysis and subsequently onto either malate or lactate in the cytoplasm through MDH1 or LDH activity, respectively. If deuterium-labeled malate is imported into the mitochondria, the deuterium label can be scrambled by fumarate hydratase due to the symmetry of fumarate. Deuterium-labeled malate/fumarate can generate deuterium-labeled citrate. FIGS. 2G and 2H. Fractional m+1 enrichment of malate (G) or citrate (H) in control and Acly-edited ESCs cultured in medium containing [4-²H] glucose. FIG. 2I. Quantification of the lactate over pyruvate ratio in control and Acly-edited ESCs. Data are means±SD, n=3 independent replicates. Significance was assessed using unpaired two-tailed Student's t-test (A, B), by two-way ANOVA (C) or one-way ANOVA (D, G-I) with Sidak's multiple comparisons post-test relative to controls (*, P<0.05; **, P<0.01; ***, P<0.001; ****, P<0.0001).

FIGS. 3A-3D: Engagement of the non-canonical TCA cycle is cell-state dependent. FIG. 3A. Western blot comparing expression of myogenesis markers MYOG and MYH3 between proliferating (Prolif) and 100% confluent (Conf) myoblasts and myotubes differentiated for 3, 5 or 7 days. FIG. 3B Fractional enrichment of malate m+2 relative to citrate m+2 (Mal+2/Cit+2) derived from [U-¹³C] glucose in proliferating and confluent myoblasts and myotubes differentiated for 3, 5 or 7 days. FIGS. 3C and 3D Measurement of the glucose-derived Mal+2/Cit+2 ratio (C) or steady-state levels of TCA cycle metabolites (expressed as the log₂ fold change relative to shRenilla, D) in myoblasts (top) and myotubes (bottom). Myoblasts and myotubes expressing doxycycline-inducible shRNAs targeting Acly (shAcly-1 and shAcly-2), Aco2 (shAco2-1 and shAco2-2) or Renilla luciferase (shRen, used as a control) were cultured on doxycycline for two or four days, respectively, to induce shRNA expression. Data (B, C) are means±SD, n=3. Significance was assessed in comparison to proliferating myoblasts (B) or shRen-expressing control myoblasts (C, top) or myotubes (C, bottom) by one-way ANOVA with Sidak's multiple comparisons post-test (n.s., P>0.05; *, P<0.05; ***, P<0.001; ****, P<0.0001).

FIGS. 4A-4J: Exit from naïve pluripotency requires engagement of the non-canonical TCA cycle. FIGS. 4A and 4B. Fractional enrichment of malate m+2 relative to citrate m+2 (Mal+2/Cit+2) derived from [U-¹³C] glucose (A) or m+1 citrate derived from [4-²H] glucose (B) in ESCs subjected to 2i/LIF withdrawal for the indicated times. FIGS. 4C and 4D. Assessment of the [U-¹³C] glucose-derived Mal+2/Cit+2 ratio (C) or steady-state levels of TCA cycle metabolites (D) in naïve, 2i-adapted control (Ctrl) and Acly-edited (ACLY-1 and ACLY-2) ESCs. Steady-state levels are represented as the fold change (expressed in log₂) relative to control cells. FIGS. 3E and 3F. Assessment of the [U-¹³C] glucose-derived Mal+2/Cit+2 ratio (E) and steady-state levels of TCA cycle metabolites (F) in control and Acly-edited ESCs subjected to 2i/LIF withdrawal for 40 h. Steady-state levels are represented as the fold change (expressed in log₂) relative to control cells. FIG. 3G. Steady-state levels of citrate, malate and aspartate in control and Acly-edited ESCs subjected to 2i/LIF withdrawal for the indicated times. FIG. 3H. Relative viability (measured by PI exclusion) of control and Acly-edited ESCs that have been maintained in the naïve pluripotent state (+2i/LIF, left) or have exited from pluripotency for 40 h (−2i/LIF, right). i, Representative histogram of GFP intensity (left) and quantification of GFP mean fluorescence intensity (MFI; right) encoded by the Rex1::GFPd2 reporter in ESCs subjected to 2i/LIF withdrawal for 40 h in the presence of vehicle or 50 μM BMS-303141 (ACLi). ESCs maintained in the naïve, ground state of pluripotency (+2i/LIF) are included as a control. FIG. 3J. Quantification of alkaline phosphatase positive (AP+) colonies representing control and Acly-edited ESCs that failed to exit from the naïve pluripotent state. 2i-adapted ESCs were subjected to 2i/LIF withdrawal for 40 h and then resceded at clonal density into medium containing 2i/LIF. Data are means±SD, n=4 (H) or n=3 independent replicates for all other experiments. In A-C, E and H-J, significance was assessed in comparison to the 0 h timepoint (A-B, E) or control cells (C, H, J) by one-way ANOVA with Sidak's multiple comparisons post-test or in the indicated comparisons (I) with one-way ANOVA with Tukey's multiple comparisons post-test. In G, significance was assessed relative to control cells at each timepoint with stars colored according to comparison by two-way ANOVA with Sidak's multiple comparisons post-test (n.s., P>0.05; *, P<0.05; **, P<0.01; ***, P<0.001; ****, P<0.0001).

FIG. 5: Metabolic gene essentiality correlations across cancer cell lines. Heatmap depicting hierarchical clustering of pairwise genetic essentiality correlations of core metabolic pathway genes derived from four GO terms: tricarboxylic acid cycle, canonical glycolysis, one-carbon metabolic process and fatty-acyl-CoA metabolic process. Genes are color coded to the left of the heatmap according to the GO term. TCA cycle genes are highlighted (red) in the dendrogram.

FIGS. 6A-6F: Effect of ACL inhibition on ¹³C labeling of TCA cycle metabolites. FIG. 6A. Two-dimensional network diagram representing gene essentiality score correlations between TCA cycle genes and their top co-dependencies. The strength of the correlation between genes is represented by both the length and thickness of the connecting edge. FIGS. 6B and 6C. Fractional enrichment of citrate (left) and malate (right) in three non-small cell lung cancer (NSCLC) cell lines cultured in medium containing [U-¹³C] glucose (B) or [U-¹³C] glutamine (C) and treated with vehicle or 50 μM BMS-303141 (ACLi) for 24 h. FIG. 6D. Schematic depicting [U-¹³C] asparagine labeling of aspartate and citrate in cells expressing guinea pig asparaginase (ASNasc). Asparagine-derived aspartate will generate m+4 labeled citrate. Top, m+4 labeled citrate metabolized via the canonical TCA cycle will lose two labeled carbons as CO₂, ultimately regenerating citrate that retains two labeled carbons (m+2). Bottom, m+4 labeled citrate metabolized by ACL will yield m+4 labeled oxaloacetate that will ultimately regenerate m+4 labeled citrate. FIGS. 6E and 6F. Fractional labeling of aspartate (left) and citrate (right) (E) or citrate m+2 relative to citrate m+4 (Cit+2/Cit+4) (F) in ASNase-expressing 143B human osteosarcoma cells cultured in medium containing [U-¹³C] asparagine and treated with vehicle or 50 μM ACLi for 24 h. Data are means±SD, n=3 independent replicates. Significance was assessed in comparison to vehicle treatment by two-way ANOVA with Sidak's multiple comparisons post-test (B-C, E) or using unpaired two-tailed Student's 1-test (F) (n.s., P>0.05; *, P<0.05; **, P<0.01; ***, P<0.001; ****, P<0.0001).

FIGS. 7A-7J. ACO2 and ACL disruption in embryonic stem cells. FIGS. 7A and 7B Immunoblot of clonal mouse embryonic stem cells (ESCs) in which CRISPR/Cas9-mediated editing was used to target either a non-genic region of chromosome 8 (Ctrl) and Acly (ACLY-1 and ACLY-2) (A) or Aco2 (ACO2-1 and ACO2-2) (B). FIG. 7C. Fractional enrichment of malate m+2 relative to citrate m+2 (Mal+2/Cit+2) in control and Aco2-edited ESCs cultured with [U-¹³C] glucose. FIG. 7D. Steady-state levels of TCA cycle metabolites in Aco2-edited ESCs. Levels are represented as the fold change (expressed in log₂) relative to Ctrl. FIGS. 6E, 6F, 6H, and 6I. Fractional m+1 enrichment of NADH (E), lactate (F), fumarate (H) and succinate (I) in control and Acly-edited ESCs cultured in medium containing [4-²H] glucose. FIG. 7G. Schematic depicting deuterium transfer from [4-²H] glucose first onto malate in the cytoplasm then onto TCA cycle metabolites in the mitochondria. FIG. 7J. The baseline oxygen consumption rate (OCR) in control and Acly-edited ESCs normalized to protein content. Twelve technical replicates were averaged for each of three independent experiments. Data are means±SD, n=3 independent replicates unless otherwise noted. Significance was assessed in comparison to control cells by one-way ANOVA with Sidak's multiple comparisons post-test (n.s., P>0.05; *, P<0.05; **, P<0.01; ****, P<0.0001).

FIGS. 8A-8J: SLC25A1 and MDH1 contribute to TCA cycle metabolism in embryonic stem cells. FIGS. 8A and 8B. Immunoblot of clonal mouse embryonic stem cells (ESCs) in which CRISPR/Cas9-mediated editing was used to target either a non-genic region of chromosome 8 (Ctrl) and Slc25a1 (SLC25A1-1 and SLC25A1-2) (A) or Mdh1 (MDH1-1and MDH1-2) (B). FIGS. 8C and 8D. Fractional m+1 enrichment of malate (Mal), fumarate (Fum), aspartate (Asp) and citrate (Cit) in control (Ctrl) and Slc25a1-edited ESCs (C) or Mdh1-edited ESCs (D) cultured in medium containing [4-²H] glucose. FIGS. 8E and 8F. Fractional m+2 enrichment of citrate, fumarate, malate and aspartate (E) or malate m+2 relative to citrate m+2 (Mal+2/Cit+2) (F) derived from [U-¹³C] glucose in control and Slc25a1-edited ESCs. FIGS. 8G and 8H. Fractional m+2 enrichment of citrate, fumarate, malate and aspartate (G) or Mal+2/Cit+2 (H) derived from [U-¹³C] glucose in control and Mdh1-edited ESCs. FIGS. 8I and 8J. Steady-state levels of TCA cycle metabolites in Slc25a1-edited ESCs (I) or Mdh1-edited ESCs (J). Levels are represented as the fold change (expressed in log₂) relative to chromosome 8-targeted control cells. Data are means±SD, n=3 independent replicates. Significance was assessed in comparison to control cells by one-way ANOVA (F, H) or two-way ANOVA (C-E, G) with Sidak's multiple comparisons post-test (*, P<0.05; **, P<0.01; ***, P<0.001;****, P<0.0001).

FIGS. 9A-9E: Effect of myogenic differentiation on ¹³C-glucose labeling to TCA cycle intermediates. FIG. 9A Fractional labeling of citrate (left) and malate (right) in proliferating and confluent myoblasts and myotubes differentiated for 3, 5 or 7 days cultured in medium containing [U-¹³C] glucose. FIG. 9B Fractional m+1 enrichment from [4-²H] glucose of malate (Mal), fumarate (Fum), aspartate (Asp) and citrate (Cit) in myoblasts and myotubes differentiated for 5 days. FIG. 9C Western blot comparing expression of ACL and ACO2 in C2C12 cells expressing doxycycline-inducible shRNAs targeting Acly (shAcly-1 and shAcly-2), Aco2 (shAco2-1 and shAco2-2) or Renilla luciferase (shRen, used as a control). Cells were cultured on doxycycline for two days to induce shRNA expression. FIGS. 9D and 9E. Fractional m+2 enrichment of citrate (left) and malate (right) in myoblasts (D) or myotubes (E) expressing doxycycline-inducible shRNAs targeting Acly, Aco2 or Renilla luciferase cultured in medium containing [U-¹³C] glucose. Myoblasts and myotubes were cultured on doxycycline for two or four days, respectively, to induce shRNA expression. Data are means±SD, n=3 independent replicates. In A, significance was assessed using one-way ANOVA with Sidak's multiple comparisons post-test to compare total metabolite fraction labeled relative to proliferating myoblasts. In remaining panels, significance was assessed in comparison to myoblasts by two-way ANOVA (B) or shRen-expressing myoblasts (D) or myotubes (E) by one-way ANOVA with Sidak's multiple comparisons post-test (n.s., P>0.05; *, P<0.05; **, P<0.01; ***, P<0.001; ****, P<0.0001).

FIGS. 10A-10F: Transcriptional profiles associated with TCA cycle choice. FIG. 10A. Gene set enrichment analysis showing that genes positively correlated with fractional enrichment of malate m+2 relative to citrate m+2 (Mal+2/Cit+2) derived from [U-¹³C] glucose in 68 non-small cell lung cancer (NSCLC) are enriched for KEGG citric acid (TCA) cycle-associated genes. FIG. 10B. RNA-seq of TCA cycle-associated genes in C2C12 myoblasts and myotubes differentiated for 5 days. Levels are represented as the fold change (expressed in log₂) relative to the row mean. RNA-seq was performed on n=3 independently derived samples. FIGS. 10C and 10D. Fractional m+2 enrichment of citrate (Cit), fumarate (Fum), malate (Mal) and aspartate (Asp) (C) or Mal+2/Cit+2 (D) derived from [U-¹³C] glucose in C2C12 myoblasts following treatment with vehicle (Ctrl), 5 mM dichloroacetic acid (DCA) or 10 μM UK-5099 (MPCi) for 24 h. FIGS. 10E and 10F. Fractional m+2 enrichment of citrate, fumarate, malate and aspartate (E) or Mal+2/Cit+2 (F) derived from [U-¹³C] glucose in mouse embryonic stem cells (ESCs) following treatment with vehicle, 5 mM DCA or 10 μM MPCi for 24 h. Data are means±SD, n=3 independent samples. In B-F, Significance was assessed in comparison to myoblasts (B) or vehicle treatment (C-F) by two-way ANOVA (C, E) or one-way ANOVA (D, F) check these call outs with Sidak's multiple comparisons post-test (n.s., P>0.05; *, P<0.05; **, P<0.01; ***, P<0.001; ****, P<0.0001).

FIGS. 11A-11M: ACL loss blunts exit from naïve pluripotency. FIG. 11A. Experimental setup for conversion to the naïve pluripotent state and exit from naïve pluripotency. Mouse embryonic stem cells (ESCs) cultured in serum and leukemia inhibitory factor (LIF) are a heterogenous population of ESCs that can be converted to the naïve, ground state of pluripotency by addition of MEK and GSK3β inhibitors (2i) in either serum replete (scrum/LIF+2i, D-F) or scrum-free (2i/LIF, G-I) media formulations. Transition to serum-frec medium lacking 2i/LIF (−2i/LIF) induces exit from the naïve pluripotent state, enabling ESCs to gain differentiation competence. FIG. 11B. qRT-PCR of pluripotency-associated (Nanog, Esrrb, Klf2, Rex1 and Oct4) and early differentiation-associated (Fgf5, Otx2 and Sox1) genes in 2i/LIF-cultured ESCs subjected to 2i/LIF withdrawal for 12, 24 or 40 h. Levels are represented as the fold change (expressed in log₂) relative to naïve, 2i/LIF-cultured ESCs (0 h). FIG. 11C. Quantification of alkaline phosphatase (AP)-positive colonies representing ESCs that failed to exit from the pluripotent state. 2i/LIF-cultured ESCs were subjected to 2i/LIF withdrawal for 0, 12, 24 or 40 h and then reseeded at clonal density into medium containing 2i and LIF. FIGS. 11D-11F Fractional labeling of citrate (Cit), malate (Mal) and aspartate (Asp) in serum/LIF+2i-cultured ESCs incubated with [U-¹³C] glucose (D), [U-¹³C] glutamine (E) or [4-²H] glucose (F) subjected to exit from pluripotency for the indicated times. FIG. 11G Fractional enrichment of glucose-derived malate m+2 relative to citrate m+2 (Mal+2/Cit+2) in 2i/LIF-cultured ESCs subjected to 2i/LIF withdrawal for the indicated times. FIGS. 11H and 11I. Fractional labeling of citrate, malate and aspartate in 2i/LIF-cultured ESCs cultured in medium containing [U-¹³C] glucose (H) or [U-¹³C] glutamine (I) subjected to 2i/LIF withdrawal for the indicated times. FIG. 11J. Immunoblot of polyclonal ESCs in which CRISPR/Cas9-mediated editing was used to target cither a non-genic region of chromosome 8 (sgChr8) or Tcf7l1 (sgTcf7l1). FIG. 11K. qRT-PCR of pluripotency-associated (Nanog, Esrrb, and Rex1) and early differentiation-associated (Sox1) genes in control and Tcf7l1-edited ESCs adapted to the naïve, ground state and subjected to 2i/LIF withdrawal for the indicated times. Levels are represented as the fold change (expressed in log₂) relative to chromosome 8-targeted control cells in the naïve, ground state (0 h). FIGS. 11L and 11M Fractional labeling of citrate (left) and malate (right) (L) and glucose-derived Mal+2/Cit+2 ratio (M) in chromosome 8-targeted control or Tcf7l1-edited ESCs cultured in medium containing [U-¹³C] glucose subjected to 2i/LIF withdrawal for the indicated times. Data are means±SD, n=3 independent replicates. In D-E, H, I and L, significance was assessed using one-way ANOVA (D-E, H-I) or two-way ANOVA (L) with Sidak's multiple comparisons post-test to compare total metabolite fraction labeled relative to the 0 h timepoint (D-E, H-I) or control cells (L). In remaining panels, significance was assessed relative to the 0 h timepoint using one-way ANOVA (C, F-G) or chromosome 8-targeted control cells at each time point using two-way ANOVA (K, M) with Sidak's multiple comparisons post-test (n.s., P>0.05; *, P<0.05; **, P<0.01; ***, P<0.001;****, P<0.0001).

FIGS. 12A-12K: Acetate does not reverse the effects of ACL loss on exit from pluripotency. FIGS. 12A and 12B. Fractional labeling of citrate (Cit), malate (Mal) and aspartate (Asp) in control and Acly-edited ESCs cultured in medium containing [U-¹³C] glucose (A) or [U-¹³C] glutamine (B) following 40 h of 2i/LIF withdrawal. FIG. 12C Western blot showing expression of ACSS2, the enzyme that converts acetate to acetyl-CoA in the cytosol, in naïve, ground state ESCs subjected to 2i/LIF withdrawal for the indicated times. FIG. 12D Fractional labeling of palmitate in control and Acly-edited ESCs cultured in medium containing [U-¹³C] acetate following 40 h of 2i/LIF withdrawal. Each bar represents one independent sample. FIG. 12E. Western blot comparing levels of acetylation (ac) at indicated histone lysine residues in control and Acly-edited ESCs subjected to 2i/LIF withdrawal for 40 h in the presence of vehicle or 5 mM sodium acetate. FIG. 12F Relative viability (measured by PI exclusion) of control and Acly-edited ESCs subjected to 2i/LIF withdrawal for 40 h in the presence of vehicle or 5 mM sodium acetate. FIG. 12G. qRT-PCR of pluripotency-associated (Nanog, Esrrb and Rex1) and early differentiation-associated (Sox1) genes in control and Acly-edited ESCs subjected to 2i/LIF withdrawal for 40 h. Levels are represented as the fold change (expressed in log₂) relative to chromosome 8-targeted control cells. FIG. 12H. Alkaline phosphatase (AP) staining of colony formation assay representing control and Acly-edited ESCs that failed to exit the naïve pluripotent state. 2i-adapted ESCs were subjected to 2i/LIF withdrawal for 40 h and then resceded at clonal density into medium containing 2i/LIF. FIG. 12I. qRT-PCR of pluripotency-associated genes in control and Acly-edited ESCs subjected to 2i/LIF withdrawal for 40 h in the presence of vehicle or 5 mM sodium acetate. FIG. 12J. Mean fluorescence intensity (MFI) of GFP encoded by the Rex1::GFPd2 reporter in ESCs subjected to 2i/LIF withdrawal for 40 h in the presence of DMSO or 50 μM BMS-303141 (ACLi) and vehicle or 5 mM sodium acetate. FIG. 12K. Quantification of AP-positive colonies representing control and Acly-edited ESCs that failed to exit from the pluripotent state. ESCs were subjected to 2i/LIF withdrawal for 40 h in the presence of vehicle or 5 mM sodium acetate prior to resceding at clonal density into medium containing 2i and LIF. Data are means±SD, n=5 (J), n=4 (F) or n=3 (all other experiments) independent replicates. In A-B, significance was assessed using one-way ANOVA with Sidak's multiple comparisons post-test to compare total metabolite fraction labeled relative to control cells. In remaining panels, significance was assessed relative to control cells (F, K) or DMSO treatment (J) by two-way ANOVA with Sidak's multiple comparisons post-test (n.s., P>0.05; *, P<0.05; **, P<0.01; ***, P<0.001; ****, P<0.0001).

FIGS. 13A-13I: Effect of SLC25A1 and MDH1 loss in exit from naïve pluripotency. FIGS. 13A and 13B Relative viability (measured by PI exclusion) of control and Slc25a1-edited (left) and Mdh1-edited (right) ESCs maintained in the naïve pluripotent state (+2i/LIF, 13A) or subjected to 2i/LIF withdrawal for 40 h (−2i/LIF, 13C). FIG. 13C. Steady-state levels of TCA cycle metabolites in control and Slc25a1-edited (left) and Mdh1-edited (right) ESCs subjected to 2i/LIF withdrawal for 40 h. Steady-state levels are represented as the fold change (expressed in log₂) relative to control cells. FIGS. 13D and 13E. Measurement of relative O-propargyl-puromycin (OP-puro) mean fluorescence intensity (MFI) in control, Slc25a1-edited, Acly-edited and Mdh1-edited ESCs that have been maintained in the naïve pluripotent state (13D) or subjected to 2i/LIF withdrawal for 40 h (13E). Dotted line represents OP-puro MFI following cycloheximide (CHX) treatment as a control. FIGS. 13F and 13G. Population doublings of control, Slc25a1-edited, Acly-edited and Mdh1-edited ESCs that have been maintained in the naïve pluripotent state (13F) or subjected to 2i/LIF withdrawal for 40 h (13G). FIGS. 13H and 13I. qRT-PCR of pluripotency-associated (Nanog, Esrrb and Rex1) and early differentiation-associated (Sox1) genes in control and Slc25a1-edited (13H) and Mdh1-edited (13I) ESCs subjected to 2i/LIF withdrawal for 40 h. Data are means±SD, n=3 independent samples. Significance was assessed in comparison to control cells by one-way ANOVA with Sidak's multiple comparisons post-test (n.s., P>0.05; ****, P<0.0001).

FIGS. 14A-14C: Mode of TCA cycle engagement regulates cell fate. FIG. 14A Population doublings of control and Aco2-edited ESCs cultured in metastable (serum/LIF) conditions. FIG. 14B Cumulative population doublings over the indicated passages of control and Aco2-edited ESCs upon conversion to the naïve, ground state of pluripotency via addition of MEK and GSK3β inhibitors (+2i). FIG. 14C qRT-PCR of pluripotency-associated genes at the indicated passages in control and Aco2-edited ESCs following addition of 2i. Gene expression at every passage was normalized to passage 0 (p0). Data are means±SD, n=1 (14B) or n=3 (14A, 14C) independent replicates. Significance was assessed in comparison to control cells by one-way ANOVA with Sidak's multiple comparisons post-test (a) or relative to control cells at each timepoint with stars colored according to comparison by two-way ANOVA with Sidak's multiple comparisons post-test (c) (n.s., P>0.05; *, P<0.05; **, P<0.01; ****, P<0.0001).

FIGS. 15A-15D: Acetate supplementation enhances processivity in mouse embryonic stem cells. Mouse embryonic stem cells (ESCs) cultured in serum and leukemia inhibitory factor (LIF; S/L) are a heterogenous population of ESCs that can be converted to the naïve, ground state of pluripotency by addition of MEK and GSK3B inhibitors (+2i). FIG. 15A Fractional enrichment of malate m+2 relative to citrate m+2 (malate m+2/citrate m+2) derived from [U-¹³C] glucose in ESCs cultured under S/L conditions or converted to the naïve, ground state of pluripotency (+2i). FIG. 15B. The malate m+2/citrate m+2 ratio in S/L-cultured ESCs following supplementation with vehicle or 5 mM sodium acetate for 24 h. FIG. 15C. Quantification of GFP mean fluorescence intensity (MFI) encoded by the Rex1::GFPd2reporter in ESCs induced to exit from the naïve, ground state of pluripotency via withdrawal of 2i and LIF for 40 h in the presence of vehicle or 5 mM sodium acetate. FIG. 15D. Quantification of alkaline phosphatase positive (AP+) colonies representing ESCs that failed to exit from the naïve pluripotent state. 2i-adapted ESCs were subjected to 2i and LIF withdrawal for 40 h in the presence of vehicle or 5 mM sodium acetate and then reseeded at clonal density into medium containing 2i and LIF.

### 6. DETAILED DESCRIPTION

The tricarboxylic acid (TCA) cycle is a central pathway of carbon metabolism. The inventors of the present disclosure observed that genes encoding enzymes of the TCA cycle segregate into two functional modules with a clear division at the point of citrate metabolism. Enzymes involved in cytosolic citrate metabolism, including citrate lyase (ACL), which catabolizes citrate to acetyl-CoA for fatty acid synthesis, and citrate/malate mitochondrial antiporter (SLC25A1), were functionally correlated with enzymes mediating citrate production in mitochondria, evidencing that these pathways are linked. Indeed, isotope tracing revealed that in cancer cell lines and in embryonic stem cells, a considerable pool of citrate flows through ACL, generating cytosolic oxaloacetate; oxaloacetate is then metabolized to malate, which returns to mitochondria through SLC25A1 to regenerate citrate pools.

Utilization of this alternative cycle (“non-canonical”) versus the canonical TCA cycle was further found to be associated with cell state and regulation of pluripotency.

The present disclosure provides methods for modulating (e.g., maintaining) pluripotency and self-renewal property of cells (e.g., stem cells) by blocking the non-canonical TCA cycle (e.g., by using an inhibitor of ATP citrate lyase (ACL) or acetate), and kits and compositions relating thereto. It relates to the discovery that blocking the non-canonical TCA cycle using ACL inhibitors or acetate prevented stem cells from exiting the pluripotent state.

Non-limiting embodiments of the invention are described by the present specification and Examples.

For purposes of clarity of disclosure and not by way of limitation, the detailed description is divided into the following subsections:
6.1. Definitions;
6.2. Methods for modulating pluripotency of cells;
6.3. Cell population and compositions;
6.4. Kits; and
6.5. Exemplary embodiments.

### 6.1. Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “in vitro” refers to an artificial environment and to processes or reactions that occur within an artificial environment. In vitro environments exemplified, but are not limited to, test tubes and cell cultures.

As used herein, the term “a population of cells” or “a cell population” refers to a group of at least two cells. In non-limiting examples, a cell population can include at least about 10,at least about 100, at least about 200, at least about 300, at least about 400, at least about 500,at least about 600, at least about 700, at least about 800, at least about 900, at least about 1000cells. The population may be a pure population comprising one cell type, such as a population of pluripotent cells. Alternatively, the population may comprise more than one cell type, for example a mixed cell population, e.g., a mixed population of pluripotent and non-pluripotent cells.

As used herein, the term “self-renewal” refers to a process by which undifferentiated cells divide to make more undifferentiated cells. For example, stem cells divide to make more stem cells, perpetuating the stem cell pool throughout life. Self-renewal is division with maintenance of the undifferentiated state. This requires cell cycle control and often maintenance of stemness. Self-renewal programs involve networks that balance proto-oncogenes (promoting self-renewal), gate-keeping tumor suppressors (limiting self-renewal), and care-taking tumor suppressors (maintaining genomic integrity). These cell-intrinsic mechanisms are regulated by cell-extrinsic signals from the niche, the microenvironment that maintains stem cells and regulates their function in tissues.

As used herein, the term “stem cell” refers to a cell with the ability to divide for indefinite periods in culture and to give rise to specialized cells. A human stem cell refers to a stem cell that is from a human. A mouse stem cell refers to a stem cell that is from a mouse.

As used herein, the term “embryonic stem cell line” refers to a population of embryonic stem cells which have been cultured under in vitro conditions that allow proliferation without differentiation for up to days, months to years.

As used herein, the term “pluripotent”, “pluripotency” or “pluripotent state” refers the properties of a cell, i.e., an ability to differentiate into a variety of tissues or organs. In certain embodiments, a pluripotent cell has the ability to differentiate into all three embryonic germ layers: endoderm, mesoderm, and ectoderm. Pluripotent cells typically have the potential to divide extensively. In certain embodiments, a pluripotent cell expresses one or more genes selected from the group consisting of Nanog, Esrrb, Klf2, Rex1, and Oct4. In certain embodiments, a pluripotent cell does not express early differentiation-associated markers including, without any limitation, Fgf5, Otx2, Oct6, Brachyury, Gata4, and Sox1.

As used herein, the term “progenitor cell” refers to cells that have greater developmental potential, i.e., a cellular phenotype that is more primitive (e.g., is at an earlier step along a developmental pathway or progression) relative to a cell which it can give rise to by differentiation. Often, progenitor cells have significant or very high proliferative potential. Progenitor cells can give rise to multiple distinct cells having lower developmental potential, i.e., differentiated cell types, or to a single differentiated cell type, depending on the developmental pathway and on the environment in which the cells develop and differentiate.

As used herein, the term “induced pluripotent stem cell” or “iPSC” refers to a type of pluripotent stem cell, similar to an embryonic stem cell, formed by the introduction of certain embryonic genes (such as a OCT4, SOX2, and KLF4 transgenes) (see, for example, Takahashi and Yamanaka Cell 126, 663-676 (2006), herein incorporated by reference) into a somatic cell, for examples, CI 4, C72, and the like. Non-limiting exemplary somatic cells that can be reprogrammed into iPSCs include keratinocytes, fibroblasts, hepatocytes, and gastric epithelial cells.

As used herein, the term “somatic cell” refers to any cell in the body other than gametes (egg or sperm); sometimes referred to as “adult” cells.

As used herein, the term “somatic (adult) stem cell” refers to a relatively rare undifferentiated cell found in many organs and differentiated tissues with a limited capacity for both self-renewal (in the laboratory) and differentiation. Such cells vary in their differentiation capacity, but it is usually limited to cell types in the organ of origin.

As used herein, the term “cell culture” refers to a growth of cells in vitro in an artificial medium for research or medical treatment.

As used herein, the term “medium” or “culture medium” interchangeably refers to a liquid that covers cells in a culture vessel, such as a Petri plate, a multi-well plate, and the like, and contains nutrients to nourish and support the cells. Culture medium may also include growth factors added to produce desired changes in the cells.

### 6.2. Methods

The present disclosure provides methods for modulating pluripotency and self-renewal property of cells. In certain embodiments, the methods maintain pluripotency of cells. In certain embodiments, the methods maintain self-renewal property of cells. In certain embodiments, the methods prevent cells from existing pluripotent state. In certain embodiments, the pluripotent state is reinforced intrinsically by the activity and coordination of the pluripotency gene regulatory network (which includes, without any limitation, Nanog, Sox2, Oct4, etc.) and that this intrinsic regulation is supported by extrinsic culture conditions.

In certain embodiments, the cells are pluripotent cells. In certain embodiments, the cells are stem cells. In certain embodiments, the cells are progenitor cells. In certain embodiments, the stem cells are selected from the group consisting of pluripotent stem cells, embryonic stem cells (ESCs), induced pluripotent stem cells (iPSCs), parthenogenetic stem cells, primordial germ cell-like pluripotent stem cells, epiblast stem cells, F-class pluripotent stem cells, somatic stem cells, cancer stem cells, any other cell capable of lineage specific differentiation, and combinations thereof. In certain embodiments, the cells are ESCs.

In certain embodiments, the cells are human cells or mouse cells. In certain embodiments, the cells are human ESCs or mouse ESCs.

In certain embodiments, blocking the non-canonical TCA cycle comprises contacting the cells with an ATP citrate lyase (ACL) inhibitor. In certain embodiments, the ACL inhibitor is a synthetic ACL inhibitor, a natural ACL inhibitor, or a combination thereof.

Non-limiting examples of suitable synthetic ACL inhibitors that can be used with the present invention include BMS-303141, sulfoximine, 2,2-difluorocitrate, expoxide, SB-201076, SB-204990, medica 16, 3-thiadicarboxylic acid, ETC-1002, ETC-1002-CoA, 2-hydroxy-narylbenzenesulfonamide, furan carboxylate derivatives, derivatives thereof, and combinations thereof. In certain embodiments, the ACL inhibitor is BMS-303141 or a derivative thereof.

Non-limiting examples of suitable natural ACL inhibitors that can be used with the present invention include (-)-hydroxycitric acid, 2-chloro-1,3,8-trihydroxy-6-methylanthracen-9 (10H)-one, antimycins A2, antimycins A8, purpurone, radicicol, cucurbitacin B, derivatives thereof, and combinations thereof.

Non-limiting examples of suitable ACL inhibitor that can be used with the present invention are disclosed in Granchi, Eur J Med Chem. 2018 Sep. 5; 157:1276-1291, the contents of which are incorporated herein by reference in their entireties. In certain embodiments, the cells are contacted with the ACL inhibitor at a concentration of at least about 10 μM, at least about 20 μM, at least about 30 μM, at least about 40 μM, at least about 50 μM or more. In certain embodiments, the cells are contacted with the ACL inhibitor at a concentration of between about 10 μM and about 200 μM, between about 10 μM and about 150 μM, between about 10 μM and about 100 μM, between about 10 μM and about 80 μM, between about 10 μM and about 60 μM, between about 10 μM and about 50 μM, between about 20 μM and about 100 μM, between about 20 μM and about 80 μM, between about 20 μM and about 60 μM, between about 40 μM and about 100 μM, between about 40 μM and about 80 μM, between about 40 μM and about 60 μM. In certain embodiments, the cells are contacted with the ACL inhibitor at a concentration of between about 40 μM and about 50 μM. In certain embodiments, the cells are contacted with the ACL inhibitor at a concentration of about 50 μM. In certain embodiments, the ACL inhibitor is BMS-303141.

In certain embodiment, blocking the non-canonical TCA cycle comprises contacting the cells with acetate. In certain embodiment, blocking the non-canonical TCA cycle comprises contacting the cells with sodium acetate.

In certain embodiments, the cells are contacted with acetate at a concentration of at least about 1 mM, at least about 2 mM, at least about 3 mM, at least about 4 mM, at least about 5 mM or more. In certain embodiments, the cells are contacted with acetate at a concentration of between about 1 mM and about 20 mM, between about 1 mM and about 15 mM, between about 1 mM and about 10 mM, between about 1 mM and about 8 mM, between about 1 mM and about 6 mM, between about 1 mM and about 5 mM, between about 2 mM and about 10 mM, between about 2 mM and about 8 mM, between about 2 mM and about 6 mM, between about 4 mM and about 10 mM, between about 4 mM and about 8 mM, between about 4 mM and about 6 mM. In certain embodiments, the cells are contacted with acetate at a concentration of between about 4 mM and about 5 mM. In certain embodiments, the cells are contacted with acetate at a concentration of about 5 mM. In certain embodiments, acetate is sodium acetate.

In certain embodiments, the cells are contacted with the ACL inhibitor or acetate for at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 25 hours, at least about 26 hours, at least about 27 hours, at least about 28 hours, at least about 29 hours, at least about 30 hours, at least about 31 hours, at least about 32 hours, at least about 33 hours, at least about 34 hours, at least about 35 hours, at least about 36 hours, at least about 37 hours, at least about 38 hours, at least about 39 hours, at least about 40 hours, at least about 41 hours, at least about 42 hours, at least about 43 hours, at least about 44 hours, at least about 45 hours, at least about 46 hours, at least about 47 hours, or at least about 48 hours. In certain embodiments, the cells are contacted with the ACL inhibitor or acetate for at least about 40 hours. In certain embodiments, the cells are contacted with the ACL inhibitor or acetate for at least about 23 hours, at least about 24 hours, or at least about 25 hours. In certain embodiments, the cells are contacted with the ACL inhibitor for at least about 40 hours. In certain embodiments, the cells are contacted with the ACL inhibitor for about 23 hours, about 24 hours, about 25 hours, or about 40 hours.

### 6.3. Cell Population and Compositions

The present disclosure provides a plurality of cells (e.g., pluripotent cells, stem cells, progenitor cells) produced by the methods described herein (e.g., a method disclosed in Section 5.2). The present disclosure also provides a composition comprising the plurality of cells disclosed herein.

In certain embodiments, the cells are pluripotent cells. In certain embodiments, the cells are stem cells. In certain embodiments, the cells are progenitor cells. In certain embodiments, the stem cells are selected from the group consisting of pluripotent stem cells, embryonic stem cells (ESCs), induced pluripotent stem cells (iPSCs), parthenogenetic stem cells, primordial germ cell-like pluripotent stem cells, epiblast stem cells, F-class pluripotent stem cells, somatic stem cells, cancer stem cells, any other cell capable of lineage specific differentiation, and combinations thereof. In certain embodiments, the cells are ESCs.

In certain embodiments, the pluripotency of the cells is maintained, after blocking the non-canonical tricarboxylic acid (TCA) cycle of the cells. In certain embodiments, the self-renewal property of the cells is maintained, after blocking the non-canonical tricarboxylic acid (TCA) cycle of the cells.

In certain embodiments, the composition comprises from about 1×10⁴ to about 1×10¹⁰, from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ of the presently disclosed cells (e.g., pluripotent cells, stem cells, progenitor cells) produced by the methods described herein (e.g., a method disclosed in Section 6.2).

### 6.4. Kits

The present disclosure provides for modulating pluripotency and self-renewal property of cells. In certain embodiments, the kits maintain pluripotency of cells. In certain embodiments, the kits maintain self-renewal property of the cells. In certain embodiments, the kits prevent cells from existing pluripotent state.

In certain embodiments, the kits comprise an agent that blocks non-canonical tricarboxylic acid (TCA) cycle, and a plurality of cells.

In certain embodiments, the cells are pluripotent cells. In certain embodiments, the cells are stem cells. In certain embodiments, the cells are progenitor cells. In certain embodiments, the stem cells are selected from the group consisting of pluripotent stem cells, embryonic stem cells (ESCs), induced pluripotent stem cells (iPSCs), parthenogenetic stem cells, primordial germ cell-like pluripotent stem cells, epiblast stem cells, F-class pluripotent stem cells, somatic stem cells, cancer stem cells, any other cell capable of lineage specific differentiation, and combinations thereof. In certain embodiments, the cells are ESCs.

In certain embodiments, the cells are human cells or mouse cells. In certain embodiments, the cells are human ESCs or mouse ESCs. In certain embodiments, the cells are a combination of the cells disclosed herein.

In certain embodiments, the agent that blocks the non-canonical TCA cycle is an ACL inhibitor. In certain embodiments, the ACL inhibitor is a synthetic ACL inhibitor, a natural ACL inhibitor, or a combination thereof.

Non-limiting examples of suitable synthetic ACL inhibitors that can be used with the present invention include BMS-303141, sulfoximine, 2,2-difluorocitrate, expoxide, SB-201076, SB-204990, medica 16, 3-thiadicarboxylic acid, ETC-1002, ETC-1002-CoA, 2-hydroxy-narylbenzenesulfonamide, furan carboxylate derivatives, derivatives thereof, and combinations thereof. In certain embodiments, the ACL inhibitor is BMS-303141.

Non-limiting examples of suitable natural ACL inhibitors that can be used with the present invention include (−)-hydroxycitric acid, 2-chloro-1,3,8-trihydroxy-6-methylanthracen-9(10H)-one, antimycins A2, antimycins A8, purpurone, radicicol, cucurbitacin B, derivatives thereof, and combinations thereof.

Non-limiting examples of suitable ACL inhibitor that can be used with the present invention are disclosed in Granchi, Eur J Med Chem. 2018 Sep. 5; 157:1276-1291, the contents of which are incorporated herein by reference in their entireties.

In certain embodiments, the kits comprise instructions for modulating the pluripotency of cells. In certain embodiments, the kits comprise instructions for maintaining the pluripotency of cells. In certain embodiments, the kits comprise instructions for maintaining the self-renewal property of the cells.

In certain embodiments, the instructions comprise contacting the cell with an agent that blocks TCA cycle as described by the methods of the present disclosure (see Section 6.2 of the present disclosure).

### 6.5. Exemplary Embodiments

A1. In certain non-limiting embodiments, the present disclosure provides a method for maintaining pluripotency of cells, comprising blocking non-canonical tricarboxylic acid (TCA) cycle of the cells.

A2. In certain non-limiting embodiments, the present disclosure provides a method for maintaining self-renewal property of cells, comprising blocking non-canonical tricarboxylic acid (TCA) cycle of the cells

A3. The foregoing method of A1 or A2, wherein blocking the non-canonical TCA cycle comprises contacting the cells with an ATP citrate lyase (ACL) inhibitor.

A4. The foregoing method of A3, wherein the ACL inhibitor is a synthetic ACL inhibitor, a natural ACL inhibitor, or a combination thereof.

A5. The foregoing method of A4, wherein the synthetic ACL inhibitor is selected from the group consisting of BMS-303141, sulfoximine, 2,2-difluorocitrate, expoxide, SB-201076, SB-204990, medica 16, 3-thiadicarboxylic acid, ETC-1002, ETC-1002-CoA, 2-hydroxy-narylbenzenesulfonamide, furan carboxylate derivatives, derivatives thereof, and combinations thereof.

A6. The foregoing method of A4 or A5, wherein the synthetic ACL inhibitor is BMS-303141.

A7. The foregoing method of A4, wherein the natural ACL inhibitor is selected from the group consisting of (−)-hydroxycitric acid, 2-chloro-1,3,8-trihydroxy-6-methylanthracen-9(10H)-one, antimycins A2, antimycins A8, purpurone, radicicol, cucurbitacin B, derivatives thereof, and combinations thereof.

A8. The foregoing method of any one of A3-A7, wherein the concentration of the ACL inhibitor is between about 10 μM and about 100 μM.

A9. The foregoing method of any one of A3-A8, wherein the concentration of the ACL inhibitor is about 50 μM.

A10. The foregoing method of any one of A3-A9, wherein the cells are contacted with the ACL inhibitor for at least about 12 hours.

A11. The foregoing method of any one of A3-A10, wherein the cells are contacted with the ACL inhibitor for about 24 hours.

A12. The foregoing method of A1 or A2, wherein blocking the non-canonical TCA cycle comprises contacting the cells with acetate.

A13. The foregoing method of any one of A1-A12, wherein the cells are pluripotent cells, stem cells, progenitor cells, or a combination thereof.

A14. The foregoing method of A13, wherein the stem cells are selected from the group consisting of pluripotent stem cells, embryonic stem cells (ESCs), induced pluripotent stem cells (iPSCs), parthenogenetic stem cells, primordial germ cell-like pluripotent stem cells, epiblast stem cells, F-class pluripotent stem cells, somatic stem cells, cancer stem cells, any other cell capable of lineage specific differentiation, and combinations thereof. A15. The foregoing method of claim any one of A1-A14, wherein the cells are ESCs. A16. The foregoing method of A15, wherein the ESCs are human ESCs or mouse ESCs.

B1. In certain non-limiting embodiments, the present disclosure provides a plurality of cells, wherein pluripotency of the cells is maintained, after blocking the non-canonical tricarboxylic acid (TCA) cycle of the cells.

B2. In certain non-limiting embodiments, the present disclosure provides a plurality of cells, wherein self-renewal property of the cells is maintained, after blocking the non-canonical tricarboxylic acid (TCA) cycle of the cells.

B3. The foregoing plurality of cells of B1 or B2, wherein blocking the non-canonical TCA cycle comprises contacting the cells with an ATP citrate lyase (ACL) inhibitor.

B4. The foregoing plurality of cells of any one of B1-B3, wherein the ACL inhibitor is a synthetic ACL inhibitor, a natural ACL inhibitor, or a combination thereof.

B5. The foregoing plurality of cells of B4, wherein the synthetic ACL inhibitor is selected from the group consisting of BMS-303141, sulfoximine, 2,2-difluorocitrate, expoxide, SB-201076, SB-204990, medica 16, 3-thiadicarboxylic acid, ETC-1002, ETC-1002-CoA, 2-hydroxy-narylbenzenesulfonamide, furan carboxylate derivatives, derivatives thereof, and combinations thereof.

B6. The foregoing plurality of cells of B4 or B5, wherein the synthetic ACL inhibitor is BMS-303141.

B7. The foregoing plurality of cells of B4, wherein the natural ACL inhibitor is selected from the group consisting of (−)-hydroxycitric acid, 2-chloro-1,3,8-trihydroxy-6-methylanthracen-9 (10H)-one, antimycins A2, antimycins A8, purpurone, radicicol, cucurbitacin B, derivatives thereof, and combinations thereof.

B8. The foregoing plurality of cells of any one of B3-B7, wherein the concentration of the ACL inhibitor is between about 10 μM and about 100 μM.

B9. The foregoing plurality of cells of any one of B3-B8, wherein the concentration of the ACL inhibitor is about 50 μM.

B10. The foregoing plurality of cells of any one of B3-B9, wherein the cells are contacted with the ACL inhibitor for at least about 12 hours.

B11. The foregoing plurality of cells of any one of B3-B10, wherein the cells are contacted with the ACL inhibitor for about 24 hours.

B12. The foregoing plurality of cells of B1 or B2, wherein blocking the non-canonical TCA cycle comprises contacting the cells with acetate.

B13. The foregoing plurality of cells of any one of B1-B12, wherein the cells are pluripotent cells, stem cells, progenitor cells, or a combination thereof.

B14. The foregoing plurality of cells of B13, wherein the stem cells are selected from the group consisting of pluripotent stem cells, embryonic stem cells (ESCs), induced pluripotent stem cells (iPSCs), parthenogenetic stem cells, primordial germ cell-like pluripotent stem cells, epiblast stem cells, F-class pluripotent stem cells, somatic stem cells, cancer stem cells, any other cell capable of lineage specific differentiation, and combinations thereof.

B15. The foregoing plurality of cells of any one of B1-B14, wherein the cells are ESCs.

B16. The foregoing plurality of cells of B15, wherein the ESCs are human ESCs or mouse ESCs.

C1. In certain non-limiting embodiments, the present disclosure provides a composition comprising the cells of any one of B1-B16.

D1. In certain non-limiting embodiments, the present disclosure provides a kit for maintaining pluripotency or self-renewal property of cells, comprising: an agent that blocks non-canonical tricarboxylic acid (TCA) cycle, and a plurality of cells.

D2. The foregoing kit of D1, wherein the cells are pluripotent cells, stem cells, progenitor cells, or a combination thereof.

D3. The foregoing kit of D2, wherein the stem cells are selected from the group consisting of pluripotent stem cells, embryonic stem cells (ESCs), induced pluripotent stem cells (iPSCs), parthenogenetic stem cells, primordial germ cell-like pluripotent stem cells, epiblast stem cells, F-class pluripotent stem cells, somatic stem cells, cancer stem cells, any other cell capable of lineage specific differentiation, and combinations thereof.

D4. The foregoing kit of any one of D1-D3, wherein the cells are ESCs.

D5. The foregoing kit of D4, wherein the ESCs are human ESCs or mouse ESCs.

D6. The foregoing kit of any one of D1-D5, wherein the agent is an ATP citrate lyase (ACL) inhibitor.

D7. The foregoing kit of D6, wherein the ACL inhibitor is a synthetic ACL inhibitor, a natural ACL inhibitor, or a combination thereof.

D8. The foregoing kit of D7, wherein the synthetic ACL inhibitor is selected from the group consisting of BMS-303141, sulfoximine, 2,2-difluorocitrate, expoxide, SB-201076, SB-204990, medica 16, 3-thiadicarboxylic acid, ETC-1002, ETC-1002-CoA, 2-hydroxy-narylbenzenesulfonamide, furan carboxylate derivatives, derivatives thereof, and combinations thereof.

D9. The foregoing kit of D7 or D8, wherein the synthetic ACL inhibitor is BMS-303141.

D10. The foregoing kit of D7, wherein the natural ACL inhibitor is selected from the group consisting of (−)-hydroxycitric acid, 2-chloro-1,3,8-trihydroxy-6-methylanthracen-9(10H)-one, antimycins A2, antimycins A8, purpurone, radicicol, cucurbitacin B, derivatives thereof, and combinations thereof.

D11. The foregoing kit of any one of D6-D10, wherein the concentration of the ACL inhibitor is between about 10 μM and about 100 μM.

D12. The foregoing kit of any one of D6-D11, wherein the concentration of the ACL inhibitor is about 50 μM.

D13. The foregoing kit of any one of D6-D12, wherein the kit further comprises instructions of contacting the cells with the ACL inhibitor for at least about 12 hours.

D14. The foregoing kit of any one of D6-D13, wherein the kit further comprises instructions of contacting the cells with the ACL inhibitor for about 24 hours. D15. The foregoing kit of any one of D1-D5, wherein the agent is acetate.

### 7. EXAMPLE

The presently disclosed subject matter will be better understood by reference to the following Example, which is provided as exemplary of the presently disclosed subject matter, and not by way of limitation.

### 7.1. Summary

The tricarboxylic acid (TCA) cycle is a central hub of cellular metabolism, oxidizing nutrients to generate reducing equivalents for energy production and critical metabolites for biosynthetic reactions. Despite the importance of TCA cycle products for cell viability and proliferation, mammalian cells display diversity in TCA cycle activity. How this diversity is achieved, and whether it is critical for establishing cell fate, remains poorly understood. Here, the present disclosure identified a non-canonical TCA cycle required for changes in cell state. Genetic co-essentiality mapping revealed a cluster of genes sufficient to compose a biochemical alternative to the canonical TCA cycle, wherein mitochondrially derived citrate exported to the cytoplasm is metabolized via ATP citrate lyase (ACL), ultimately regenerating mitochondrial oxaloacetate to complete this non-canonical TCA cycle. Manipulating expression of ACL or the canonical TCA cycle enzyme aconitase 2 in mouse myoblasts and embryonic stem cells (ESCs) revealed that changes in TCA cycle configuration accompany cell fate transitions. During exit from pluripotency, ESCs switch from canonical to non-canonical TCA cycle metabolism; accordingly, blocking the non-canonical TCA cycle prevents cells from exiting pluripotency. These results established a context-dependent alternative to the traditional TCA cycle and reveal that appropriate TCA cycle engagement is required for changes in cell state.

### 7.2. Methods

Metabolic coessentiality analysis and network modeling. To obtain metabolic gene essentiality scores, the present disclosure analyzed CERES gene dependency values from the DepMap Portal Project Achilles^{46,47} 20Q2 release in which 18,119 genes were perturbed by genome-wide loss of function CRISPR screens in 769 human cancer cell lines. The present disclosure utilized two gene lists to perform our analysis. To focus on an unbiased set of metabolic genes corresponding to well-defined metabolic pathways, the present disclosure created a gene set of 122 genes derived from four gene ontology (GO) terms^{48,49}: tricarboxylic acid cycle (GO: 0006099), canonical glycolysis (GO: 0061621), 1-carbon metabolic process (GO: 0006730) and fatty-acyl-CoA metabolic process (GO: 0035337). To focus more specifically on TCA cycle-centered analysis, the present disclosure used a list of 27 core TCA cycle genes and then identified the top 10 correlates of these TCA genes using Pearson correlation coefficients from DepMap gene essentiality scores above a minimum threshold (r>0.25). Next, the present disclosure identified the top 5 correlates of this expanded list again above a minimum threshold (r>0.25), resulting in a list of 115 TCA-cycle associated genes.

To examine genetic co-dependency in these gene lists, Pearson correlation coefficients were calculated between metabolic gene essentiality scores across the 769 human cancer cell lines surveyed to generate a correlation matrix heatmap of codependent gene modules. To create the heatmap, the correlations were hierarchically clustered with the UPGMA algorithm using the scipy.cluster.hierarchy.linkage function from the SciPy Python package⁵⁰, with the method argument set to ‘average’. The heatmap was graphed using the Seaborn Python package (https://seaborn.pydata.org/citing.html). To visualize codependent gene modules as a network diagram, the present disclosure utilized the Python package NetworkX (http://networkx.org). Genes with no correlation partners or with low correlation scores (r<0.25) were filtered out, and spring model layouts were generated using the method ‘neato’ from the Python package PyGraphviz (http://pygraphviz.github.io). Graph edges were weighted according to the strength of pairwise gene correlations and the final network diagram was created using the NetworkX draw function. Gene clusters with less than 3 members were removed.

Gene expression correlation. NSCLC cell line isotope tracing data was obtained from Chen et al., 2019 (ref¹⁸). Gene expression data was obtained from the DepMap Cancer Cell Line Encyclopedia⁵¹. For the 68 cell lines present in both datasets, expression of each gene was correlated with the fractional enrichment of malate m+2 relative to citrate m+2 (Mal+2/Cit+2) derived from [U-¹³C] glucose. Genes were ranked by correlation with Mal+2/Cit+2, and gene set enrichment analysis⁵² of the gene set KEGG citric acid (TCA) cycle-associated genes (KEGG_CITRATE_CYCLE_TCA_CYCLE; M3985) was performed using GSEAPreranked version 4 with default parameters. Data were exported and graphed in GraphPad Prism version 9.

Cell culture. Mouse embryonic stem cells (ESCs) were previously generated from C57BL/6×129S4/SvJae F1 male embryos²⁹. Rex1::GFPd2 ESCs³⁸ were a kind gift from Austin Smith, University of Exeter, United Kingdom. All other cell lines were obtained from ATCC. ESCs were maintained on gelatin-coated plates in the following media: serum/LIF, serum/LIF+2i, or 2i/LIF. Serum/LIF medium contained knockout DMEM (catalog no. 10829018; Thermo Fisher Scientific) supplemented with 10% fetal bovine serum (FBS; Gemini), 0.1 mM 2-mercaptoethanol, 2 mM L-glutamine and 1,000 U mlL⁻¹ LIF (Gemini). To generate serum/LIF+2i maintenance medium, serum/LIF medium was supplemented with 3 μM CHIR99021 (Stemgent) and 1 μM PD0325901 (Stemgent) (2i). 2i/LIF medium contained a 1:1 mix of DMEM/F-12 (catalog no. 11320033; Gibco) and Neurobasal medium (catalog no. 21103049; Gibco) including N-2 supplement (catalog no. 17502048; Gibo), B-27 supplement (catalog no. 17504044; Gibco), 2-mercaptoethanol, 2 mM L-glutamine, LIF and 2i. To generate ESCs in the naïve ground state of pluripotency, serum/LIF-cultured ESCs were adapted for 3 passages to serum/LIF+2i medium or 2i/LIF medium. Adapted cells were used for a maximum of 9 passages.

For exit from naïve pluripotency, serum/LIF+2i-cultured ESC were seeded at least 24 h prior to washing with PBS and changing into medium containing a 1:1 mix of glutamine-free DMEM (catalog no. 11960051; Gibco) and Neurobasal medium including N-2 supplement, B-27 supplement, 2-mercaptoethanol and 2 mM L-glutamine at the indicated time before harvest (24 or 40 h). 2i/LIF-cultured ESCs were seeded at least 24 h prior to being washed with PBS and changed into serum-free maintenance medium without 2i or LIF at the indicated time before harvest (12, 24 or 40 h). Unless otherwise noted, cells were adapted to serum/LIF+2i culture prior to exit from naïve pluripotency.

C2C12 cells were maintained at sub-confluence as myoblasts unless otherwise noted. All myoblast experiments were performed in high glucose DMEM supplemented with 10% FBS and 4 mM L-glutamine. For differentiation into myotubes, C2C12 cells were grown to 100% confluence for three days and then washed with PBS and changed into differentiation medium composed of high glucose DMEM supplemented with 2% horse serum (catalog no. 26050070; Gibco), 4 mM L-glutamine and 100× insulin-transferrin-selenium-ethanolamine (ITS-X; catalog no. 51500056; Gibco) for the indicated length of time (3, 5 or 7 days). Differentiation medium was refreshed every day. For non-small cell lung cancer (NSCLC) cell line studies, H2170, A549, and Calu-1 cell lines were thawed and passaged in RPMI-1640 supplemented with 10% FBS before being transitioned to high glucose DMEM supplemented with 10% FBS and 4 mM L-glutamine for several passages prior to experiments. 143B cells were maintained in high glucose DMEM supplemented with sodium pyruvate, 10% FBS and penicillin-streptomycin. All cells routinely tested negative for Mycoplasma.

Generation of clonal ESC lines. Single guide (sg) RNA sequences targeting Acly, Aco2,Slc25a1, Mdh1 or a control, non-genic region on mouse chromosome 8 (chr8)⁵³ were cloned into the pSpCas9 (BB)-2A-GFP plasmid (PX458, Addgene plasmid number 48138), as previously described⁵⁴. See Supplementary Table 1 for sgRNA sequences. ESCs (4×10⁵ per condition) were electroporated using a 4D-Nucleofector (Amaxa, Lonza) with 5 μg PX458 plasmid encoding Cas9, EGFP and sgRNA sequences. After electroporation, cells were plated onto a layer of irradiated feeder mouse embryonic fibroblasts (MEFs). After 48 h, cells were dissociated with Accutase (Invitrogen) and sorted using the BD FACSAria III sorter (BD Biosciences) to enrich for GFP-positive cells. Approximately 10,000 fluorescence-activated cell sorting (FACS)-sorted GFP-positive cells per experimental condition were immediately re-seeded onto 10 cm plates (on feeder MEFs) to enable clonal growth. After 7 days, individual clones were picked and expanded (initially on feeder MEFs then on gelatin-coated tissue culture plates) and loss of target gene expression was validated by immunoblot (see below).

Generation of Tcf7l1-edited ESC lines. Cas9 cDNA from a lentiCas9-Blast plasmid (Addgene plasmid number 52962) was cloned into Piggybac (pCAGGS-IRES-Neo, a kind gift from H. Niwa, Institute of Molecular Embryology and Genetics, Kumamoto, Japan). ESCs were transfected with Piggybac plus transposase (pBase) at a 3:1 ratio using Fugene HD (catalog no. E2691; Promega). Following selection with G418 (300 μg/mL, catalog no. 10131-035, Gibco), cells were plated on feeder MEFs at single cell density to generate clonal Cas9+ ESC lines.

sgRNA sequences targeting Tcf7l1(ref⁵⁵) or a non-genic region on mouse chromosome 8 (chr8) were cloned into the pUSEPB plasmid⁵⁶ (kind gift from S. Lowe, Memorial Sloan Kettering Cancer Center, USA) as previously described⁵⁴. See Supplementary Table 1 for gRNA sequences. Lentivirus was generated by the co-transfection of sgRNA vectors with the packaging plasmids psPAX2 and pMD2.G (Addgenc) into 293T cells. Virus-containing supernatant was cleared of cellular debris by 0.45-μM filtration and was concentrated using Lenti-X (catalog no. 631231; Takara). Cas9-ESCs were exposed to concentrated viral supernatant with 6 μg/mL polybrene for 24 h before being washed, grown for 24 h in fresh medium, and subjected to antibiotic selection. Cells were expanded and loss of target gene expression was validated by immunoblot (see below).

sgRNA editing analysis. For clonal ESC lines, genomic DNA was extracted (Qiagen) and amplification of edited regions was performed from 50 ng of genomic DNA using Platinum Taq DNA Polymerase (Invitrogen), per the manufacturer's instructions. See Supplementary Table 2 for sequencing primers. Primers were optimized to produce an amplicon between 200 and 280 bp long and containing the edited locus within the first 100 bp (from either the 5′-end or 3′-end). PCR products were column purified (Qiagen) and detection of CRISPR variants from NGS reads (CRISPR sequencing) was performed by the CCIB DNA Core Facility at Massachusetts General Hospital (Cambridge, MA). Briefly, Illumina compatible adapters with unique barcodes were ligated onto each sample during library construction. Libraries were pooled in equimolar concentrations for multiplexed sequencing on the Illumina MiSeq platform with 2×150 run parameters. Upon completion of the NGS run, data were demultiplexed and subsequently entered into an automated de novo assembly pipeline, UltraCycler v1.0 (Brian Seed and Huajun Wang, unpublished). Sequenced amplicons produced for each clonal ESC line are listed in Supplementary Table 2.

Lentiviral production and infection. Renilla Luciferase-, Acly- and Aco2-targeting shRNAs were introduced into C2C12 cells to enable doxycycline-inducible expression using lentiviral LT3GEPIR⁵⁷ (see Supplementary Table 1 for shRNA sequences). Lentivirus was generated by the co-transfection of shRNA-expressing viral vectors with the packaging plasmids psPAX2 and pMD2.G (Addgenc) into 293T cells. Virus-containing supernatants were cleared of cellular debris by 0.45-μm filtration and mixed with 8 μg/mL polybrene. C2C12 cells were exposed to viral supernatants for two 24 h periods before being passaged and grown for 24 h in fresh medium and then subjected to antibiotic selection with 1 μg/mL puromycin. Cells were maintained under antibiotic selection until all cells on an uninfected control well were eliminated.

Viability assays. Serum/LIF+2i-adapted ESCs were seeded at a density of 24,000 cells per well of a 24-well plate in triplicate or quadruplicate. 24 h later, cells were washed with PBS and changed into either fresh scrum/LIF+2i medium or medium containing a 1:1 mix of glutamine-free DMEM and Neurobasal medium including N-2 supplement, B-27 supplement, 2-mercaptoethanol and 2 mM L-glutamine for 40 h. Cells were evaluated for propidium iodide (PI) on an LSRFortessa flow cytometer using FACSDiva software v8.0 (BD Biosciences). Analysis of PI exclusion was performed with FCS Express v.7.05 or FlowJo v10.8.0.

Growth curves. ESCs were seeded at a density of 40,000 cells per well of a 12-well plate. The following day, three wells of each line were counted to determine starting cell number. The remaining cells were washed with PBS and changed to either media containing serum+2i/LIF or induced to exit from naïve pluripotency as indicated above. Cells were counted 40 h later using a Beckman Coulter Multisizer 4e with a cell volume gate of 400-10,000 fl. Cell counts were normalized to starting cell number. All curves were performed at least two independent times.

Naïve pluripotency conversion growth curve. ESCs were seeded in standard culture medium (serum/LIF) in six-well plates; 48 h later, cells were counted to establish a baseline measurement of proliferation for each line under serum/LIF culturing conditions. Following this count, ESCs were seeded into serum/LIF+2i maintenance medium and passaged and counted every 48 h for 6 days (3 passages). Cumulative population doublings were assessed by summing population doublings measured at each passage. Cells were counted using a Beckman Coulter Multisizer 4e with a cell volume gate of 400-10,000 fl.

O-propargyl-puromycin (OP-puro) assay. 60 min prior to harvest all cells were washed with PBS and changed into fresh medium. For cycloheximide control samples, 10 μg/mL cycloheximide was added to wells at this time. At 30 min prior to harvest, 20 μM O-propargyl-puromycin (OP-puro, catalog no. HY-15680; MedChemExpress) was added to cells. Cells were harvested and stained with fixable viability dye (catalog no. 65-0863-14; Thermo Fisher Scientific), followed by fixation with 4% PFA in PBS and permeabilization with 0.25% Triton-X-100. Fixed and permeabilized cells were stained using Click-iT Plus Alexa Fluor 647 Picolyl Azid Toolkit (catalog no. C10643; Thermo Fisher Scientific) and AZDye 647 Picoyl Azide (catalog no. 1300-1; Click Chemistry Tools) according to manufacturer's instructions and analyzed on a LSRFortessa flow cytometer using FACSDiva software v8.0 (BD Biosciences). Analysis of OP-puro incorporation was performed with FCS Express v7.05 or FlowJo v10.8.0.

Rex1::GFPd2 analysis. On the day of analysis, cells were trypsinized and resuspended in FACS buffer (PBS+2% FBS+1 mM EDTA) containing 4,6-diamidino-2-phenylindole (DAPI, 1 μg/mL). Cells were evaluated for DAPI and GFP on an LSRFortessa flow cytometer using FACSDiva software v8.0 (BD Biosciences). Viable cells were those excluding DAPI. Rex1::GFPd2 expression was measured by GFP mean fluorescence intensity (MFI) and quantified using FCS Express v7.0.5 or FlowJo v10.8.0.

Metabolic analyses. For isotope tracing experiments, ESCs were seeded in standard culture medium in six-well plates; 24 h or 48 h later, cells were washed with PBS and changed into experimental medium containing a 1:1 mix of glutamine-free DMEM and glutamine-free Neurobasal medium including 10% dialyzed FBS, 2-mercaptoethanol, LIF and 2 mM L-glutamine with or without 2i. The next day, cells were washed with PBS and changed into medium containing a 1:1 combination of glucose-and glutamine-free DMEM and glucose-and glutamine-free Neurobasal-A medium including 10% dialyzed FBS, 2-mercaptoethanol, LIF and 2i as specified and supplemented with [¹²C] glucose (Sigma-Aldrich) and [¹²C]-glutamine (Gibco) or the labeled versions of each metabolite: [U-¹³C] glucose, [4-²H] glucose or [U-¹³C] glutamine (Cambridge Isotope Laboratories) to a final concentration of 20 mM (glucose) and 2 mM (glutamine) for 4 h prior to harvest. To analyze metabolites in serum/LIF+2i-cultured ESCs undergoing exit from pluripotency, ESCs were seeded in maintenance medium in six-well plates overnight. Either 24 or 40 h prior to harvest, cells were washed with PBS and changed into medium containing a 1:1 mix of glutamine-free DMEM and Neurobasal medium including N-2 supplement, B-27 supplement, 2-mercaptoethanol and 2 mM L-glutamine. To analyze metabolites in 2i/LIF-cultured ESCs undergoing exit from pluripotency, ESCs were seeded in serum-free maintenance medium in six-well plates overnight. Either 24 or 40 h prior to harvest, cells were washed with PBS and changed into serum-free maintenance medium without 2i or LIF. In all cases, 4 h prior to harvest, cells were washed with PBS and changed into medium containing a 1:1 combination of glucose-and glutamine-free DMEM and glucose- and glutamine-free Neurobasal-A medium including N-2 and B-27 supplements and 2-mercaptoethanol and supplemented with [¹²C] glucose and [¹²C] glutamine or the labeled versions of each metabolite to a final concentration of 20 mM (glucose) and 2 mM (glutamine).

For mass spectrometric analyses in C2C12 myoblasts and NSCLC cell lines, cells were seeded in six-well plates and medium was changed 24 h later. The next day, cells were washed with PBS and changed into medium containing glucose-and glutamine-free DMEM including 10% dialyzed FBS and supplemented with [¹²C]-glucose and [¹²C]-glutamine or the labeled versions of each metabolite to a final concentration of 20 mM (glucose) and 4 mM (glutamine) for 4 h prior to harvest. 48 h prior to harvest, myoblasts were supplemented with 1 μg/mL doxycycline to induce shRNA expression. For analysis of myotubes, cells seeded in six-well plates were grown to 100% confluence for 3 days, washed with PBS and changed to differentiation medium that was refreshed every day for 7 days. On the final day of differentiation, cells were washed with PBS and changed to experimental medium described above. For analysis of C2C12 myotube genetic hairpin lines, cells were processed as described above but medium was supplemented with 1 μg/mL doxycycline for the final four days of differentiation to induce shRNA expression. ESCs and NSCLC lines treated with inhibitors were processed as described above but medium was supplemented with DMSO, 50 μM BMS-303141 (catalog no. SML0784; Sigma), 5 mM dichloroacetate (catalog no. 3447795; Sigma) or 10 μM UK-5099 (catalog no. 4186; Tocris) for 24 h prior to harvest. At harvest, metabolites were extracted with 1 mL ice-cold 80% methanol containing 2 μM deuterated 2-hydroxyglutaratc (d-2-hydroxyglutaric-2,3,3,4,4-d₅ acid (d5-2HG)). After overnight incubation at −80° C., lysates were collected and centrifuged at 21,000 g for 20 min to remove protein. All extracts were further processed by LCMS (for analysis of NADH, lactate and pyruvate (deuterium labeling and lactate/pyruvate ratio) and succinate (deuterium labeling only) or GCMS (for all other analyses) as described below.

143B cells were plated in six-well plates; 24 h later, media was changed to DMEM supplemented with 10% dialyzed FBS, 1 mM asparagine and DMSO or 50 μM BMS-303141. After 20 h, media was changed to DMEM supplemented with 10% dialyzed FBS, 1 mM [U-¹³C]asparagine and DMSO or 50 μM BMS-303141. Cells were extracted with 300 μL 80% methanol containing Valine-D8 as an internal control. 143B extracts were further processed by LCMS, described below.

Fatty acid analyses. To analyze fatty acids in scrum/LIF+2i-cultured ESCs undergoing exit from pluripotency, ESCs were seeded in maintenance medium in six-well plates overnight. The next day, cells were washed with PBS and changed into medium containing a 1:1 mix of glutamine-free DMEM and Neurobasal medium including N-2 supplement, B-27 supplement, 2-mercaptoethanol and 2 mM L-glutamine. 24 h prior to harvest, cells were washed with PBS and changed into medium containing a 1:1 combination of glucose-and glutamine-free DMEM and glucose-and glutamine-free Neurobasal-A medium including N-2 supplement, B-27supplement, 2-mercaptoethanol, 2 mM L-glutamine and 20 mM glucose supplemented with 5 mM [1,2-¹³C] sodium acetate (Cambridge Isotope Laboratories). At harvest, lysates were collected in PBS and centrifuged at 6,800 g for 5 min to pellet cells. To isolate fatty acids, cell pellets were resuspended in 400 μl HPLC grade methanol followed by 800 μl HPLC grade chloroform and samples were vortexed for 10 min at 4° C. 300 μl HPLC grade water then added to induce phase separation. 800 μl of the bottom chloroform layer was moved to a new tube and lyophilized. Dried samples were saponified by resuspending in 1 ml of 80% methanol with 0.3 M KOH and heating at 80° C. for 1 h in a glass vial. Next, 1 ml of HPLC grade hexanes were added to the vial and briefly vortexed. 800 μl of the top hexane layer was moved to a new tube and lyophilized. Extracts were then further processed by LCMS, described below.

Gas chromatography-mass spectrometry. Extracts were dried in an evaporator (Genevac EZ-2 Elite) and resuspended by incubating with shaking at 30° C. for 2 h in 50 μl of 40 mg ml⁻¹ methoxyamine hydrochloride in pyridine. Metabolites were further derivatized by adding 80 μl of N-methyl-N-(trimethylsilyl) trifluoroacetamide+1% TCMS (Thermo Fisher Scientific) and 70 μl ethyl acetate (Sigma-Aldrich) and then incubated at 37° C. for 30 min. Samples were analyzed using an Agilent 7890A Gas Chromatograph coupled to an Agilent 5977C mass selective detector. The gas chromatograph was operated in splitless injection mode with constant helium gas flow at 1 ml min⁻¹; 1 μl of derivatized metabolites was injected onto an HP-5 ms column and the gas chromatograph oven temperature ramped from 60 to 290° C. for 25 min. Peaks representing compounds of interest were extracted and integrated using the MassHunter software v.B.08 (Agilent Technologies) and then normalized to both the internal standard (d5-2HG) peak area and protein content of triplicate samples as determined by bicinchoninic acid assay (Thermo Fisher Scientific). Steady-state metabolite pool levels were derived by quantifying the following ions: d5-2HG, 354 m/z; αKG, 304 m/z; aspartate, 334 m/z; citrate, 465 m/z; fumarate, 245 m/z; malate, 335 m/z; and succinate, 247 m/z. All peaks were manually inspected and verified relative to known spectra for each metabolite. Enrichment of [¹³C] or [²H] was assessed by quantifying the abundance of the following ions: aspartate, 334-346 m/z; citrate, 465-482 m/z; fumarate, 245-254 m/z; and malate, 335-347 m/z. Correction for natural isotope abundance was performed using IsoCor software v. 1.0 or v.2.0⁵⁸.

Liquid chromatography-mass spectrometry. Lyophilized samples were resuspended in 80% methanol in water and transferred to liquid chromatography-mass spectrometry (LCMS) vials for measurement by LCMS. Metabolite quantitation was performed using a Q Exactive HF-X Hybrid Quadrupole-Orbitrap Mass Spectrometer equipped with an Ion Max API source and H-ESI II probe, coupled to a Vanquish Flex Binary UHPLC system (Thermo Scientific). Mass calibrations were completed at a minimum of every 5 days in both the positive and negative polarity modes using LTQ Velos ESI Calibration Solution (Pierce). Polar Samples were chromatographically separated by injecting a sample volume of either 1 μL in the MS mode or 5 μL in the SIM mode into a SeQuant ZIC-PHILIC Polymeric column (2.1×150 mm 5 mM, EMD Millipore). The flow rate was set to 150 mL/min, autosampler temperature set to 10° C., and column temperature set to 30° C. Mobile Phase A consisted of 20 mM ammonium carbonate and 0.1% (v/v) ammonium hydroxide, and Mobile Phase B consisted of 100% acetonitrile. The sample was gradient eluted (% B) from the column as follows: 0-20 min.: linear gradient from 85% to 20% B; 20-24 min.: hold at 20% B; 24-24.5 min.: linear gradient from 20% to 85% B; 24.5 min.-end: hold at 85% B until equilibrated with ten column volumes. Mobile Phase was directed into the ion source with the following parameters: sheath gas=45, auxiliary gas=15, sweep gas=2, spray voltage=2.9 kV in the negative mode or 3.5 kV in the positive mode, capillary temperature=300° C., RF level=40%, auxiliary gas heater temperature=325° C. Mass detection was conducted with a resolution of 240,000 in full scan mode or 120,000 in SIM mode, with an AGC target of 3,000,000 and maximum injection time of 250 msec for the full scan mode, or 100,000 and 100 msec for the SIM mode. Metabolites were detected over mass range of 70-1050 m/z in full scan positive mode, or SIM in positive mode using a quadrupole isolation window of 0.7 m/z. Non-Polar Samples were chromatographically separated by injecting 2 μL into an Accucore Vanquish C18+column (2.1×100 mm, 1.5 μm particle size, p/n 27101-102130, Thermo Scientific). The autosampler temperature was set at 10° C. and the flow rate was 500 μL/min with the column temperature set at 50° C. The largely isocratic gradient consisted of a mixture of water/5 mM ammonium acetate as “A” and acetonitrile as “B” where between 0-6.5 min, the solvent composition was held at 60% “B”, followed by a change to 98% “B” between 6.5-6.6 min. The composition was held at 98% “B” between 6.6-9.0 min, and then returned back to starting conditions at 60% “B” between 9.0-9.1 minutes. It was then held for an additional 4.4 minutes to re-equilibrate the column for the next run. Non-polar analytes were detected in the negative polarity mode at a resolution of 240,000 in the full scan setting, using a mass range of 240-650m/z. The AGC target value was 3,000,000 with a maximum injection time of 200 ms. The chromatography peak width setting was 10 seconds (FWHM), and data were collected in profile mode. The parameters for the H-ESI source were as follows: sheath gas flow rate of 53 units, aux gas flow rate of 14 units, sweep gas flow rate of 3 units, with the spray voltage set at 3.00 kV. The funnel RF level was set at 40%, and the capillary and auxiliary gas heater temperatures were held at 300° C. and 400° C. respectively. Quantitation of all metabolites was performed using Tracefinder 4.1 (Thermo Scientific) referencing an in-house metabolite standards library using≤ 5 ppm mass error. Data from stable isotope labeling experiments includes correction for natural isotope abundance using IcoCor software v.2.2.

Oxygen consumption. Oxygen consumption rate (OCR) was measured using a Seahorse XFe96 Extracellular Flux Analyzer (Agilent Technologies). ESCs were plated on gelatin-coated tissue culture-treated XF96 96-well plates (Agilent Technologies) at 2×10⁴ cells per well in standard maintenance medium. The following day, cells were washed twice with assay medium (Seahorse XF DMEM Medium supplemented with 10 mM glucose) and changed to assay medium containing 2 mM L-glutamine for 2 h prior to the assay. Baseline measurements of OCR were obtained three times. Following the assay, protein content was determined and averaged per condition and the OCR measurements were normalized to these values. The third baseline OCR reading was averaged across all 12 replicates; averaged values from three independent experiments are shown.

Western blotting. Protein lysates were extracted in 1×RIPA buffer (Cell Signaling Technology), separated by SDS-polyacrylamide gel electrophoresis and transferred to nitrocellulose membranes (Bio-Rad). For histone blots, cell pellets were flash frozen in ethanol and resuspended in Laemmli buffer for sonication. Samples were mixed with 5% BME and 0.01% bromophenol blue prior to identical separation as for protein lysates. Membranes were blocked in 3% milk in Tris-buffered saline with 0.1% Tween 20 (TBST) or 5% BSA in TBST and incubated at 4° C. with primary antibodies overnight. After TBST washes the next day, membranes were incubated with horseradish peroxidase-conjugated secondary antibodies for at least 2 h, incubated with enhanced chemiluminescence (Thermo Fisher Scientific) and imaged with an SRX-101A X-ray Film Processor (Konica Minolta). The antibodies used (at 1:1,000) were: ACL (catalog no. 4332; Cell Signaling Technologies), ACO2 (catalog no. MA1-029; ThermoFisher), SLC25A1 (catalog no. 15235-1-AP; ProteinTech), MDH1 (catalog no. sc-166879; Santa Cruz Biotechnology), ACACA (catalog no. MAB6898; R&D Systems), AccCS1/ACSS2 (catalog no. 3658; Cell Signaling Technologies), TCF7LI (catalog no. sc-166411; Santa Cruz Biotechnology), Myogenin/MYOG (catalog no. 14-5643-82; ThermoFisher), MYH3 (catalog no. 22287-1-AP; ProteinTech), Vinculin (catalog no. V9131; Sigma), Tubulin (catalog no. T9026; Sigma-Aldrich), H3K9ac (catalog no. 9469; Cell Signaling Technology), H3K14ac (catalog no. 07-353; Millipore Sigma), H3K27ac (catalog no. 39133; Active Motif), H4K16ac, (catalog no. 39167; Active Motif), H3 (catalog no. Ab1791; Abcam), and H4 (catalog no. 07-108; Millipore Sigma). C2C12 myoblast genetic hairpin lines were maintained in medium supplemented with 1 μg/mL doxycycline for 48 h prior to protein lysate extraction to induce shRNA expression.

Colony formation assay. ESCs adapted to 2i/LIF or serum/LIF+2i were subjected to exit from pluripotency in triplicate for 12, 24 or 40 h. On the day of harvest, cells were counted and re-seeded at a density of 2,000 cells per well in technical triplicate in maintenance medium containing 2i and LIF. Medium was refreshed every 3 days. Six days after initial seeding, cells were fixed with citrate/acetone/3% formaldehyde for 30 s and stained with the Leukocyte Alkaline Phosphatase Kit (Sigma-Aldrich) according to the manufacturer's instructions. Colonies were quantified using ImageJ's particle analysis function and technical triplicates were averaged for each condition.

Quantification of gene expression. RNA was isolated from six-or twelve-well plates using TRIzol (Invitrogen) according to the manufacturer's instructions and 200 ng RNA was used for complementary DNA (cDNA) synthesis using the iScript cDNA Synthesis Kit (Bio-Rad). Quantitative real-time PCR analysis was performed in technical triplicate using QuantStudio 5 or 6 Flex (Applied Biosystems) with Power SYBR Green Master Mix (Thermo Fisher Scientific). All data were generated using cDNA from 3 independent wells for each condition. Actin was used as an endogenous control for all experiments. See Supplementary Table 1 for qRT-PCR primer sequences.

RNAseq of myoblasts and myotubes. RNA was isolated as described above and quantified using a Qubit 3.0 fluorometer. RNA-seq libraries were generated using TruSeq Stranded mRNA Library Prep Kit (catalog no. 20020594; Illumina) according to the manufacturer's instructions. Samples were pooled and sequenced at the Memorial Sloan Kettering Cancer Center Integrated Genomics Operation. RNA-seq libraries were filtered and trimmed using fastp⁵⁹ and mapped with STAR aligner⁶⁰ against the mm10 mouse genome assembly using default parameters. featureCounts⁶¹ used to calculate gene counts for input into DESeq2⁶² for quality control analysis, size normalization and variance dispersion corrections.

Statistical analyses. Prism 9 (GraphPad Software) software was used for statistical analyses except for DepMap data. Error bars, P values and statistical tests are reported in the figure legends. Statistical analyses on DepMap data were performed with Python v3.8. Experiments were performed in biological triplicate or as noted in the figure legends at least two, often many more, times.

TABLE 1
All sgRNA, shRNA and qRT-PCR primer sequences.
gRNA Sequences (complementary oligonucleotides used for cloning
sgRNAs into pX458, 5′-3′)

| | |
|---|---|
| Chr.8 control | CACCGACATTTCTTTCCCCACTGG (SEQ ID NO.: 1) |
| Chr.8 control | AAACCCAGTGGGGAAAGAAATGTC (SEQ ID NO.: 2) |
| Acly sg1 | CACCGGGGCGTACTTGAACCGGTTC (SEQ ID NO.: 3) |
| Acly sg1 | AAACGAACCGGTTCAAGTACGCCCC (SEQ ID NO.: 4) |
| Acly sg2 | CACCGGAACCGGTTCAAGTACGCCC (SEQ ID NO.: 5) |
| Acly sg2 | AAACGGGCGTACTTGAACCGGTTCC (SEQ ID NO.: 6) |
| Aco2 sg1 | CACCGGCCAACCAGGAGATCGAGCG (SEQ ID NO.: 7) |
| Aco2 sg1 | AAACCGCTCGATCTCCTGGTTGGCC (SEQ ID NO.: 8) |
| Slc25a1 sg1 | CACCGCTTCACGTATTCGGTCGGGA (SEQ ID NO.: 9) |
| Slc25a1 sg1 | AAACTCCCGACCGAATACGTGAAGC (SEQ ID NO.: 10) |
| Slc25a1 sg2 | CACCGGAGAGGACTATTGTGCGGTC (SEQ ID NO.: 11) |
| Slc25a1 sg2 | AAACGACCGCACAATAGTCCTCTCC (SEQ ID NO.: 12) |
| Mdh1 sg1 | CACCGTTGGACATCACCCCCATGAT (SEQ ID NO.: 13) |
| Mdh1 sg1 | AAACATCATGGGGGTGATGTCCAAC (SEQ ID NO.: 14) |
| Mdh1 sg2 | CACCGTCAGCCAGCTGTCGTCTTTC (SEQ ID NO.: 15) |
| Mdh1 sg2 | AAACGAAAGACGACAGCTGGCTGAC (SEQ ID NO.: 16) |
| ACACA sg1 | CACCGAAGTGTATCTGAGCTGACGG (SEQ ID NO.: 17) |
| ACACA sg1 | AAACCCGTCAGCTCAGATACACTTC (SEQ ID NO.: 18) |
| ACACA sg2 | CACCGCTCACAACGAGATTTCACTG (SEQ ID NO.: 19) |
| ACACA sg2 | AAACCAGTGAAATCTCGTTGTGAGC (SEQ ID NO.: 20) |
| sgTcf711 | CACCGCCGGGCAAGCTCATAGTATT (SEQ ID NO.: 21) |
| sgTcf711 | AAACAATACTATGAGCTTGCCCGGC (SEQ ID NO.: 22) |

XhoI/EcoRI fragment for mir30-based shRNA cloning, 5′-3′

| | |
|---|---|
| shRen | TGCTGTTGACAGTGAGCGCAGGAATTATAATGCTTAT |
| | CTATAGTGAAGCCACAGATGTATAGATAAGCATTAT |
| | AATTCCTATGCCTACTGCCTCGGA (SEQ ID NO.: 23) |
| shAcly-1 | TGCTGTTGACAGTGAGCGCCTGTATTAATCTGATTTT |
| | TAATAGTGAAGCCACAGATGTATTAAAAATCAGATT |
| | AATACAGTTGCCTACTGCCTCGGA (SEQ ID NO.: 24) |
| shAcly-2 | TGCTGTTGACAGTGAGCGCTGTAACATACAAGTGTTT |
| | AAATAGTGAAGCCACAGATGTATTTAAACACTTGTAT |
| | GTTACAATGCCTACTGCCTCGGA (SEQ ID NO.: 25) |
| shAco2-1 | TGCTGTTGACAGTGAGCGCTCACCAGATCATTCTAGA |
| | AAATAGTGAAGCCACAGATGTATTTTCTAGAATGATC |
| | TGGTGAATGCCTACTGCCTCGGA (SEQ ID NO.: 26) |
| shAco2-2 | TGCTGTTGACAGTGAGCGACAGTATGACCAAGTGATT |
| | GAATAGTGAAGCCACAGATGTATTCAATCACTTGGTC |
| | ATACTGGTGCCTACTGCCTCGGA (SEQ ID NO.: 27) |

qRT-PCR primers

| Gene | Primer (5′-to-3′) |
|---|---|
| Actin F | GCTCTTTTCCAGCCTTCCTT (SEQ ID NO.: 28) |
| Actin R | CTTCTGCATCCTGTCAGCAA (SEQ ID NO.: 29) |
| Nanog F | AAGATGCGGACTGTGTTCTC (SEQ ID NO.: 30) |
| Nanog R | CGCTTGCACTTCATCCTTTG (SEQ ID NO.: 31) |
| Esrrb F | AACAGCCCCTACCTGAACCT (SEQ ID NO.: 32) |
| Esrrb R | TGCCAATTCACAGAGAGTGG (SEQ ID NO.: 33) |
| Klf2 F | TAAAGGCGCATCTGCGTACA (SEQ ID NO.: 34) |
| Klf2 R | CGCACAAGTGGCACTGAAAG (SEQ ID NO.: 35) |
| Rex1 F | TCCATGGCATAGTTCCAACAG (SEQ ID NO.: 36) |
| Rex1 R | TAACTGATTTTCTGCCGTATGC (SEQ ID NO.: 37) |
| Klf4 F | CGGGAAGGGAGAAGACACT (SEQ ID NO.: 38) |
| Klf4 R | GAGTTCCTCACGCCAACG (SEQ ID NO.: 39) |
| Sox1 F | CCTCGGATCTCTGGTCAAGT (SEQ ID NO.: 40) |
| Sox1 R | GCAGGTACATGCTGATCATCTC (SEQ ID NO.: 41) |
| Otx2 F | GACCCGGTACCCAGACATC (SEQ ID NO.: 42) |
| Otx2 R | GCTCTTCGATTCTTAAACCATACC (SEQ ID NO.: 43) |
| Fgf5 F | AAACTCCATGCAAGTGCCAAAT (SEQ ID NO.: 44) |
| Fgf5 R | TCTCGGCCTGTCTTTTCAGTTC (SEQ ID NO.: 45) |
| Oct4 F | TGGATCCTCGAACCTGGCTA (SEQ ID NO.: 46) |
| Oct4 R | CCCTCCGCAGAACTCGTATG (SEQ ID NO.: 47) |

TABLE 2
CRISPR-sequencing primers and derived amplicons.
CRISPR-seq primers (used to amplify edited regions)

| | | |
|---|---|---|
| Acly sg1 + sg2 | F | ACCGGCAAAGAACTCCTGTACAAGT (SEQ ID NO.: 48) |
| | R | CAAGTCTGCAATCACGGCGCTC (SEQ ID NO.: 49) |
| Aco2 sg1 | F | TGCACACAGATGCTTCTGTGCCT (SEQ ID NO.: 50) |
| | R | ATAGCCATCTGGGCTGTGGCAT (SEQ ID NO.: 51) |
| Slc25al sg1 | F | TGTCCCTAGCCCCAGGCATT (SEQ ID NO.: 52) |
| | R | TTGCCGCACGCAGTCTCCTA (SEQ ID NO.: 53) |
| Slc25al sg2 | F | TTGGGATGTTCGAGTTCCTCAGC (SEQ ID NO.: 54) |
| | R | TTCCCACCTTGACAGCCTCCAT (SEQ ID NO.: 55) |
| Mdh1 sg1 | F | GCAGCTACTAAGCAAGCTATGGCA (SEQ ID NO.: 56) |
| | R | GACTTCCCAACTCACCCTGCAG (SEQ ID NO.: 57) |
| Mdh1 sg2 | F | CCAGATGTCAATCATGCCAAGGTG (SEQ ID NO.: 58) |
| | R | GTGTCATAGATCAGGTCACATCAGAC (SEQ ID NO.: 59) |
| ACACA sg1 | F | CCTGGTGAAGCTGGACCTAGAAG (SEQ ID NO.: 60) |
| | R | TCATAGAAGATGGGCCAGGGAA (SEQ ID NO.: 61) |
| ACACA sg2 | F | GCATGTCTGGCTTGCACCTAG (SEQ ID NO.: 62) |
| | R | GGAGACAGAAAGGATAGCCCTGCAT (SEQ ID NO.: 63) |

CRISPR-seq amplicons

| Line | Allele | Sequence |
|---|---|---|
| ACO2 | | |
| ACO2-1 | 1 | TGCACACAGATGCTTCTGTGCCTGCTCACTGCCACACATG |
| | | TGAGAGGGAAAAGCTGTGCCAGGCTGAGTCCCAAAGCTA |
| | | CTTCCTTCTTTGTGCCCGTAGGTTGAACCGGCCTCTTACTC |
| | | TCTCAGAGAAGATTGTATATGGACACCTGGATGACCCAGC |
| | | CAACCAGGAGATC---- |
| | | GGGGAAAGACATACCTGCGTTTACGGCCCGACCGGGTGG |
| | | CCATGCAGGATGCCACAGCCCAGATGGCTAT |
| | | (SEQ ID NO.: 64) |
| | 2 | TGCACACAGATGCTTCTGTGCCTGCTCACTGCCACACATG |
| | | TGAGAGGGAAAAGCTGTGCCAGGCTGAGTCCCAAAGCTA |
| | | CTTCCTTCTTTGTGCCCGTAGGTTGAACCGGCCTCTTACTC |
| | | TCTCAGAGAAGATTGTATATGGACACCTGGATGACCCAGC |
| | | CAACCAGGAGATC---- |
| | | AGGGGAAAGACATACCTGCGTTTACGGCCCGACCGGGTG |
| | | GCCATGCAGGATGCCACAGCCCAGATGGCTAT |
| | | (SEQ ID NO.: 65) |
| ACO2-2 | 1 | TGCACACAGATGCTTCTGTGCCTGCTCACTGCCACACATG |
| | | TGAGAGGGAAAAGCTGTGCCAGGCTGAGTCCCAAAGCTA |
| | | CTTCCTTCTTTGTGCCCGTAGGTTGAACCGGCCTCTTACTC |
| | | TCTCAGAGAAGATTGTATATGGACACCTGGATGACCCAGC |
| | | CAACCAGGA----------- |
| | | GAAAGACATACCTGCGTTTACGGCCCGACCGGGTGGCCAT |
| | | GCAGGATGCCACAGCCCAGATGGCTAT |
| | | (SEQ ID NO.: 66) |
| | 2 | TGCACACAGATGCTTCTGTGCCTGCTCACTGCCACACATG |
| | | TGAGAGGGAAAAGCTGTGCCAGGCTGAGTCCCAAAGCTA |
| | | CTTCCTTCTTTGTGCCCGTAGGTTGAACCGGCCTCTTACTC |
| | | TCTCAGAGAAGATTGTATATGGACACCTGGATGACCCAGC |
| | | CAACCAGGAGATCGAGCTGCTGGGGAAAGACATCGAGCA |
| | | GCGGGGAAAGACATACCTGCGTTTACGGCCCGACCGGGT |
| | | GGCCATGCAGGATGCCACAGCCCAGATGGCTAT |
| | | (SEQ ID NO.: 67) |
| Mdh1 | | |
| MDH1-1 | 1 | GCAGCTACTAAGCAAGCTATGGCATCTCTCTTCCATAATT |
| | | ATGACTAATTAGTGGGAACTAAATATATACAAACAAAAAT |
| | | AGGGTGGGACTTTGTGTGAGAGAGAATGCAGTAACCACC |
| | | CCGACCTTCACCATAACCGCATTGCCTGCTGTCCTTGCTCT |
| | | TTGGCAGCCCATCATTCTTGTGCTGTTGGACATCACCCCCA |
| | | TTGATGGGTGTTCTGGACGGTGTCCTGATGGAACTGCAAG |
| | | ACTGTGCCCTTCCCCTTCTGCAGGGTGAGTTGGGAAGTC |
| | | (SEQ ID NO.: 68) |
| MDH1-2 | 1 | CCAGATGTCAATCATGCCAAGGTGAAACTGCAAGGAAAG |
| | | GAAGTCGGTGTGTATGAAGCCCTG- |
| | | AAGACGACAGCTGGCTGAAGGGAGAGTTCATCACGGTAA |
| | | GAAGGATGTGAACCCTCTGAGCAGCCAGAGGAGACACAC |
| | | AGCACACTGCTGAGCTGAGTGTCTGCCAGGGCCCTGTGGG |
| | | ACCCTCAAGGACAGCTGCTGTTTTACTCTAATATATTCTTT |
| | | TGAAGTGTGCAAAAAGATGGTTTTCATTGTCTGATGTGAC |
| | | CTGATCTATGACAC |
| | | (SEQ ID NO.: 69) |
| | 2 | CCAGATGTCAATCATGCCAAGGTG----------------- |
| | | ------------------------------- |
| | | AGCTGGCTGAAGGGAGAGTTCATCACGGTAAGAAGGATG |
| | | TGAACCCTCTGAGCAGCCAGAGGAGACACACAGCACACT |
| | | GCTGAGCTGAGTGTCTGCCAGGGCCCTGTGGGACCCTCAA |
| | | GGACAGCTGCTGTTTTACTCTAATATATTCTTTTGAAGTGT |
| | | GCAAAAAGATGGTTTTCATTGTCTGATGTGACCTGATCTA |
| | | TGACAC |
| | | (SEQ ID NO.: 70) |
| Acly | | |
| ACLY-1 | 1 | CAAGTCTGCAATCACGGCGCTCCTTGAGACACCTCCAACA |
| | | AAGCAAATGCTTTCCCCTCCTCAGAGCCTGAGCTGGACAG |
| | | AAGGGCTCAGGGTGCAGGCAGAGCTTACCTGGCTGAGCA |
| | | GCCACGGGTGGTCCTGCAGCAGATGGGCCCAGTCTGTGTC |
| | | GGGAGTCACCCGGGCGTACTT----------- |
| | | GGATGGCCGAGGTGGTGCAGATGTACTTGTACAGGAGTTC |
| | | TTTGCCGGT |
| | | (SEQ ID NO.: 71) |
| | 2 | CAAGTCTGCAATCACGGCGCTCCTTGAGACACCTCCAACA |
| | | AAGCAAATGCTTTCCCCTCCTCAGAGCCTGAGCTGGACAG |
| | | AAGGGCTCAGGGTGCAGGCAGAGCTTACCTGGCTGAGCA |
| | | GCCACGGGTGGTCCTGCAGCAGATGGGCCCAGTCTGTGTC |
| | | GGGAGTCACCCGGGCGTACTTGAACCGG---- |
| | | GGATGGCCGAGGTGGTGCAGATGTACTTGTACAGGAGTTC |
| | | TTTGCCGGT |
| | | (SEQ ID NO.: 72) |
| ACLY-2 | 1 | CAAGTCTGCAATCACGGCGCTCCTTGAGACACCTCCAACA |
| | | AAGCAAATGCTTTCCCCTCCTCAGAGCCTGAGCTGGACAG |
| | | AAGGGCTCAGGGTGCAGGCAGAGCTTACCTGGCTGAGCA |
| | | GCCACGGGTGGTCCTGCAGCAGATGGGCCCAGTCTGTGTC |
| | | GGGAGTC---------------------------------- |
| | | ------------- |
| | | TACATCTGCACCACCTCGGCCATCCAGAACCGGTTCAAGT |
| | | ACTTGTACAGGAGTTCTTTGCCGGT |
| | | (SEQ ID NO.: 73) |
| Slc25a1 | | |
| SLC25A1-1 | 1 | TGTCCCTAGCCCCAGGCATTTGGTCCCCACAGGCGGCCTG |
| | | GCTGGAGGCATCGAAATCTGCATCACCTTCCCGA---- |
| | | ATACGTGAAGACGCAGCTGCAGCTAGATGAACGAGCGAA |
| | | CCCACCGCGGTACCGGGGCATCGGTAAGGAGGGACTCCC |
| | | GGGGGTGGCTAGGGGGACACTGCGGAGCAGAGGAGCTAG |
| | | GGTCTTGACCGCGCCTCCCGTAGGAGACTGCGTGCGGCAA |
| | | (SEQ ID NO.: 74) |
| | 2 | TGTCCCTAGCCCCAGGCATTTGGTCCCCACAGGCGGCCTG |
| | | GCTGGAGGCATCGAAATCTGCATCACCTTC- |
| | | GACCGAATACGTGAAGACGCAGCTGCAGCTAGATGAACG |
| | | AGCGAACCCACCGCGGTACCGGGGCATCGGTAAGGAGGG |
| | | ACTCCCGGGGGTGGCTAGGGGGACACTGCGGAGCAGAGG |
| | | AGCTAGGGTCTTGACCGCGCCTCCCGTAGGAGACTGCGTG |
| | | CGGCAA |
| | | (SEQ ID NO.: 75) |
| SLC25A1-2 | 1 | TTGGGATGTTCGAGTTCCTCAGCAACCACATGCGGGATGC |
| | | CCAAGGTAGGCTCGACAGCAGGAGAGGACTATTGT----- |
| | | ------- |
| | | GCCGGCGTGGCAGAAGCAGTGGTAGTCGTGTGCCCTATGG |
| | | AGACCATCAAGGTGAGGGATTGAGCCTTTCAGGGGTAGCC |
| | | AGGGTATCTTCGGGCTCCCAGGTTTTAATGGCATGTGCCC |
| | | CCATGGAGGCTGTCAAGGTGGGAA |
| | | (SEQ ID NO.: 76) |
| | 2 | TTGGGATGTTCGAGTTCCTCAGCAACCACATGCGGGATGC |
| | | CCAAGGTAGGCTCGACAGCAGGAGAGGAC--------- |
| | | ATTGTCTGGGTGCCGGCGTGGCAGAAGCAGTGGTAGTCGT |
| | | GTGCCCTATGGAGACCATCAAGGTGAGGGATTGAGCCTTT |
| | | CAGGGGTAGCCAGGGTATCTTCGGGCTCCCAGGTTTTAAT |
| | | GGCATGTGCCCCCATGGAGGCTGTCAAGGTGGGAA |
| | | (SEQ ID NO.: 77) |

### 7.3 Results

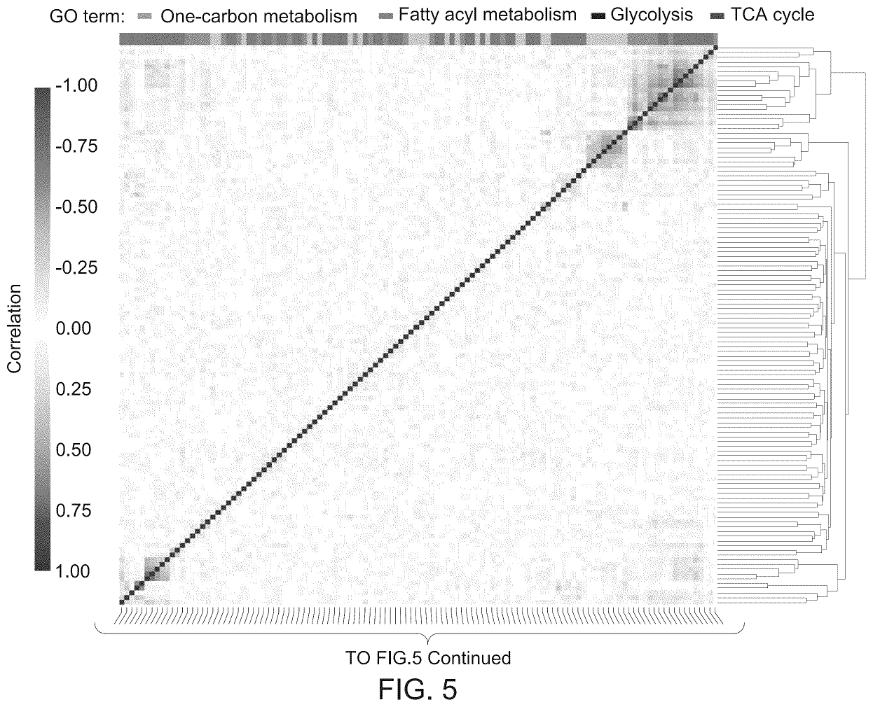

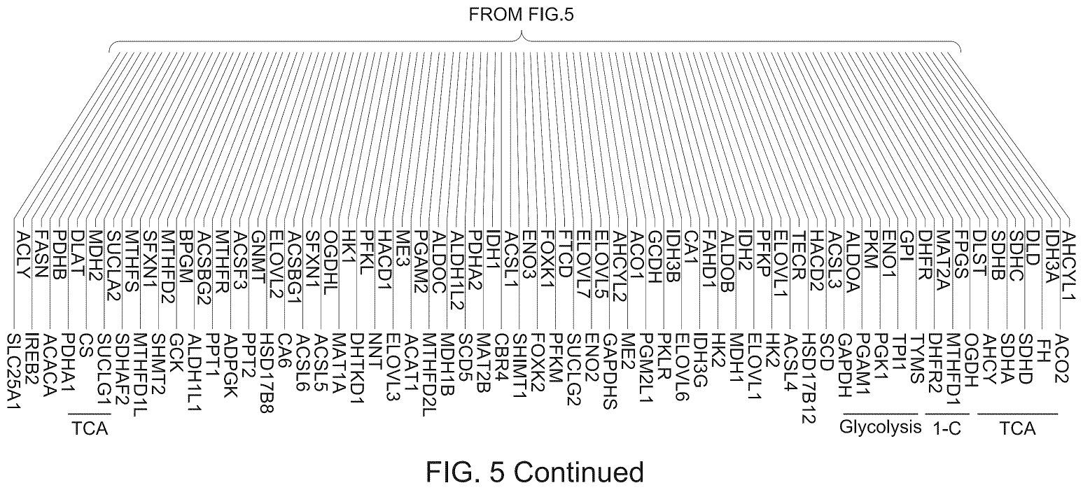

To identify metabolic networks involving TCA cycle proteins, the present disclosure analyzed gene essentiality scores generated from genome wide CRISPR screens in 769 cancer cell lines by the DepMap project¹⁵. Genes involved in glycolysis, fatty acid oxidation, 1-carbon metabolism or the TCA cycle were clustered based on pairwise correlation of gene essentiality scores (FIG. 5). The vast majority of genes were weakly correlated, perhaps reflecting inherent metabolic plasticity of cultured cells and/or variable isoform expression. Of the clusters that emerged, one contained genes involved in glycolysis and one contained genes required for 1-carbon metabolism, consistent with the notion that these genes comprise distinct functional modules (FIG. 5).

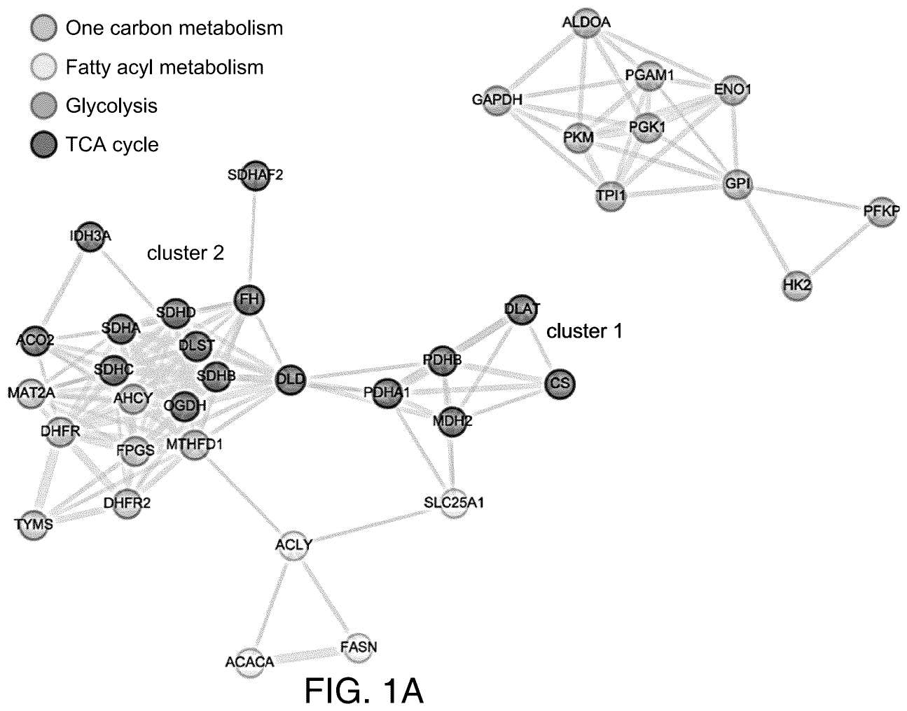

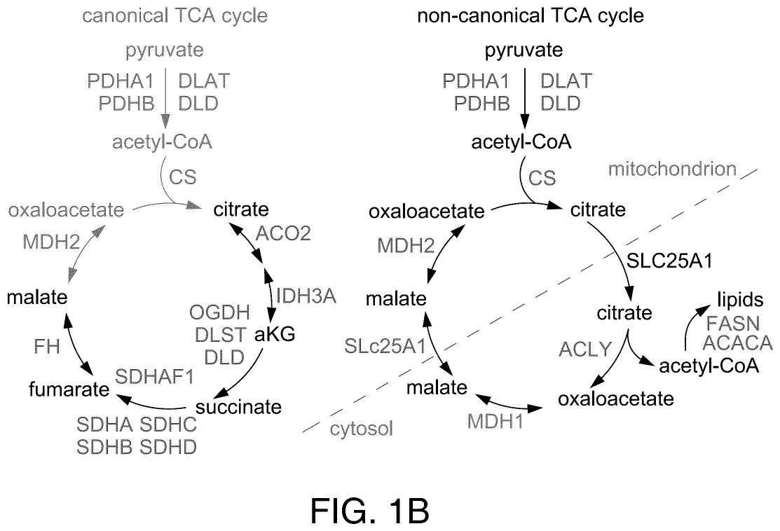

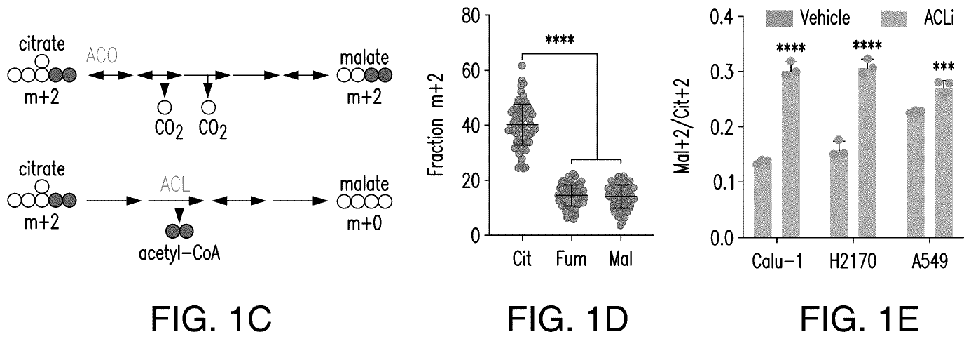

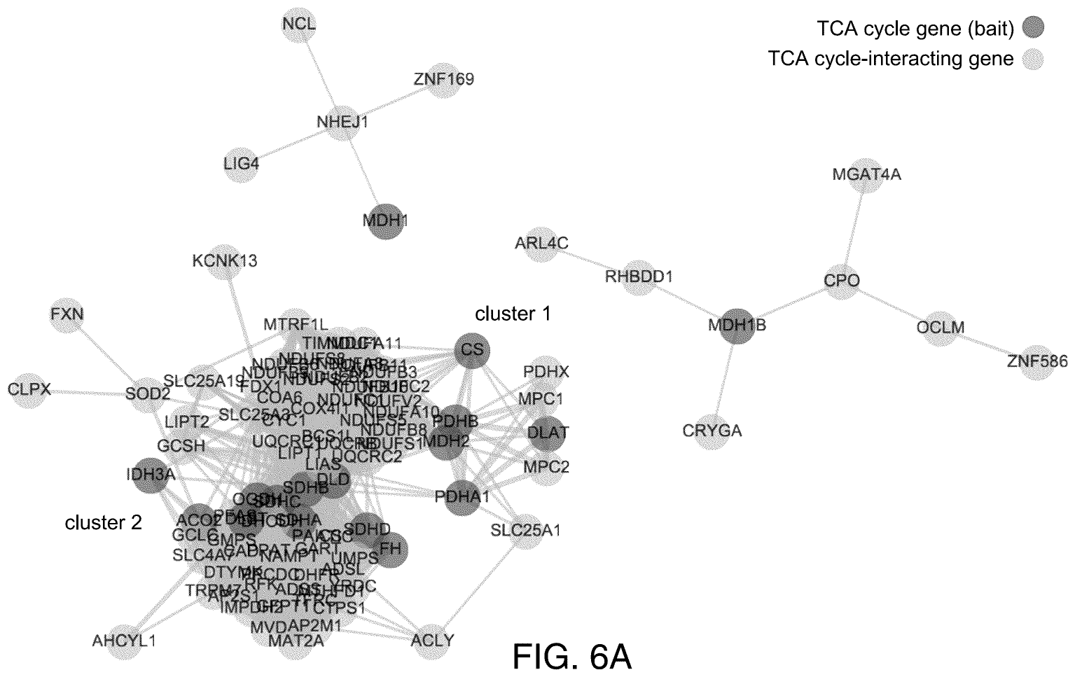

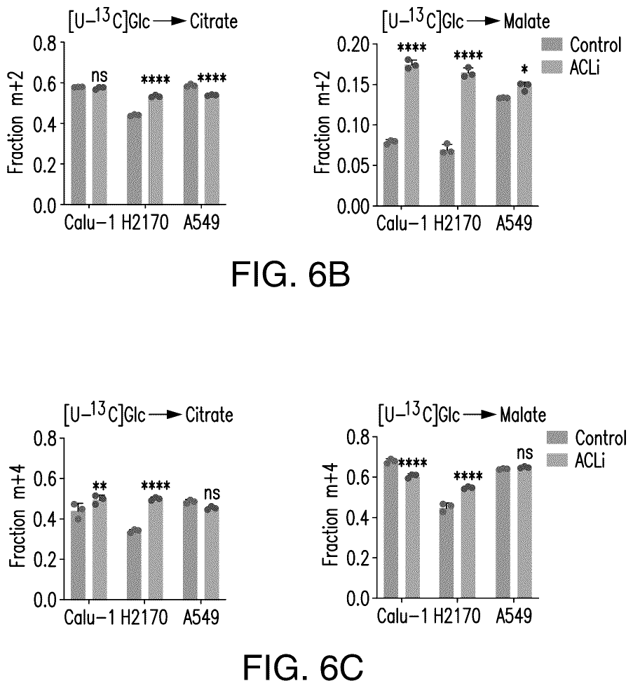

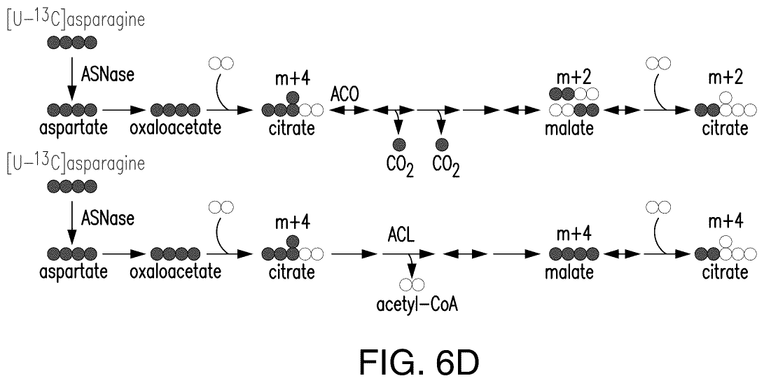

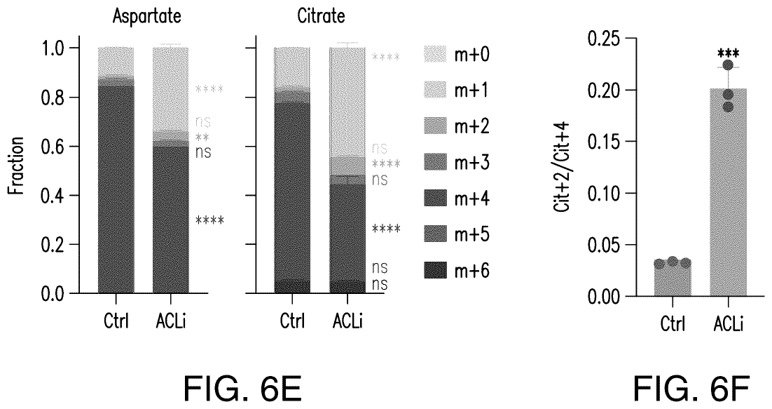

In contrast, TCA cycle-associated genes separated into two distinct clusters: one containing genes required for generation of citrate from pyruvate and oxaloacetate and one involved in the sequential oxidation of mitochondrial citrate (FIG. 5). Two-dimensional mapping of correlation distance (FIG. 1A) demonstrated that these two clusters were linked by the shared co-dependency for Dld, a subunit required for both the pyruvate dehydrogenase complex (cluster 1) and the oxoglutarate dehydrogenase complex (cluster 2). The same two clusters emerged even when analyzing the top co-dependencies of all TCA cycle genes (FIG. 6A). Further analysis of co-dependencies revealed that enzymes involved in cytosolic citrate metabolism, including the mitochondrial citrate/malate antiporter (Slc25a1) and ATP citrate lyase (Acly) correlated with enzymes involved in citrate production (FIG. 1A). The separation of genes involved in citrate production and citrate oxidation was surprising and suggested that oxidative pyruvate metabolism by the TCA cycle may not function as a single module, contrary to the canonical view of TCA cycle metabolism.

Mapping genes in each cluster onto the canonical TCA cycle pathway underscored a clear division of the TCA cycle into two segments upstream and downstream of citrate (FIG. 1B), raising the question of how cells sustain citrate production if oxidative production of oxaloacetate via the TCA cycle is not tightly linked to citrate synthesis. Strikingly, mapping genes associated with cluster 1 revealed that these enzymes comprise a coherent metabolic pathway capable of continuous oxaloacetate regeneration for citrate production (FIG. 1B). Mitochondrial citrate efflux and conversion to oxaloacetate via ACL provides the cytoplasmic acetyl-CoA required for acetylation reactions and lipid biosynthesis and is therefore well established as a critical pathway for proliferating cells^{16,17}. The co-dependency of ACL with TCA cycle enzymes suggests that ACL may also support cellular metabolic demands by forming a functional, non-canonical TCA cycle.

The route by which citrate is processed—either through aconitase in a traditional TCA cycle or through ACL in a non-canonical cycle—can be monitored by tracing of isotopically labeled glucose carbons throughout TCA cycle intermediates. Oxidative decarboxylation of glucose-derived pyruvate results in the m+2 labeled isotopologue of citrate, which when metabolized by mitochondrial aconitase (ACO2) will generate m+2 labeled isotopologues of downstream TCA cycle metabolites (FIG. 1C, top). However, if m+2 labeled citrate is exported to the cytoplasm and metabolized by ACL, then the two labeled carbons will be liberated in the form of acetyl-CoA and the remaining oxaloacetate backbone and any TCA cycle intermediates derived from it will be unlabeled (FIG. 1C, bottom). Thus, a drop off in m+2 labeling of TCA cycle intermediates downstream of citrate can reflect in part the degree to which cells engage in non-canonical TCA cycle activity.

Indeed, a striking disconnect between m+2 labeling of citrate and downstream metabolites is frequently observed in cultured cells. Isotope tracing of a panel of over 80 non-small cell lung cancer (NSCLC) lines¹⁸ revealed that while on average approximately 40% of citrate is m+2 labeled from glucose, only 15% of malate contains the m+2 label (FIG. 1D). To determine whether loss of glucose label downstream of citrate is due in part to flux through ACL, the present disclosure treated select NSCLC lines with the ACL inhibitor BMS-303141¹⁹. In all lines, ACL inhibition increased the proportion of malate containing two labeled carbons while having only a minor effect on the fraction of citrate m+2 (FIG. 6B). Glutamine anaplerosis, in which glutamine-derived carbons enter the TCA cycle as α-ketoglutarate, will also contribute to a disconnect between fractional labeling of citrate and malate²⁰. Importantly, ACL inhibition did not uniformly affect glutamine anaplerosis under these conditions, indicating that the effect of ACL on label loss downstream of citrate is not merely due to label dilution from glutamine anaplerosis (FIG. 6C). Within an individual cell line, the degree to which malate is derived from the canonical TCA cycle can be represented as the ratio of malate m+2 relative to citrate m+2 (mal+2/cit+2). ACL inhibition increased the mal+2/cit+2 ratio in all cell lines, consistent with the model that citrate metabolism via ACL represents a significant alternative to the canonical TCA cycle (FIG. 1E).

To confirm the effect of ACL inhibition on TCA cycle metabolism, the present disclosure took advantage of an orthogonal labeling strategy in which human 143B osteosarcoma cells are engineered to use asparagine as a source of TCA cycle intermediates by expression of guinea pig asparaginase 1 (gpASNase1)²¹. In this system, asparagine-derived aspartate enables production of m+4 labeled citrate, which will only lose labeled carbons if metabolized by the traditional TCA cycle (FIG. 6D). Here, ACL inhibition significantly increased the ratio of cit+2/cit+4, which reflects the degree to which citrate is regenerated through the oxidative TCA cycle (FIGS. 6E and 6F). Together, these results indicate that flux through ACL contributes to the production of TCA cycle intermediates and suggest that cancer cells employ two distinct methods for oxaloacetate recycling for continuous citrate production.

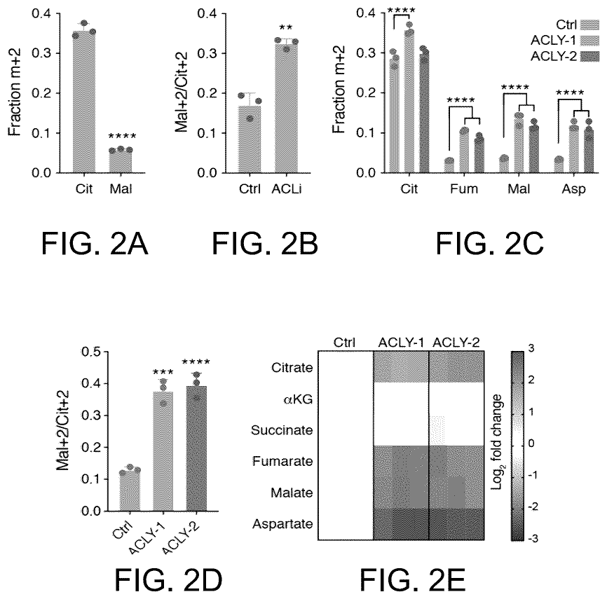

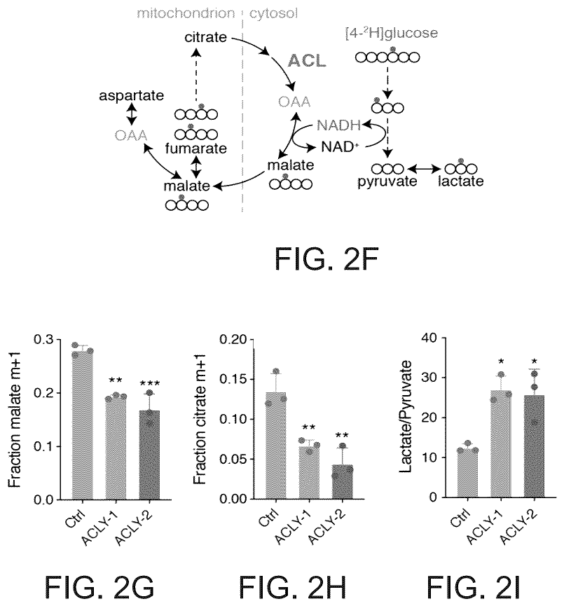

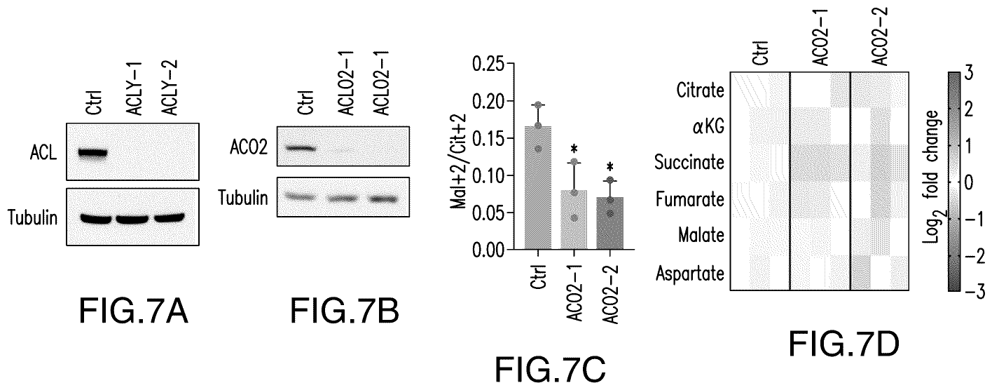

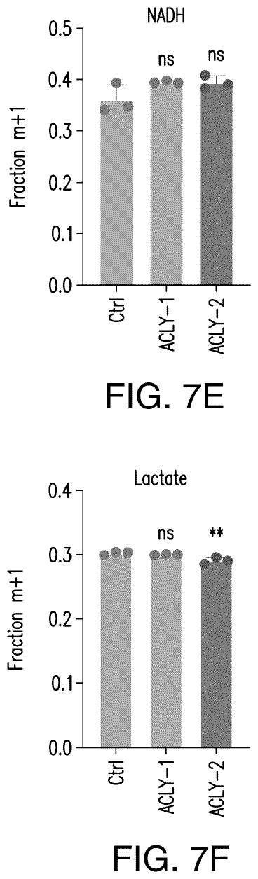

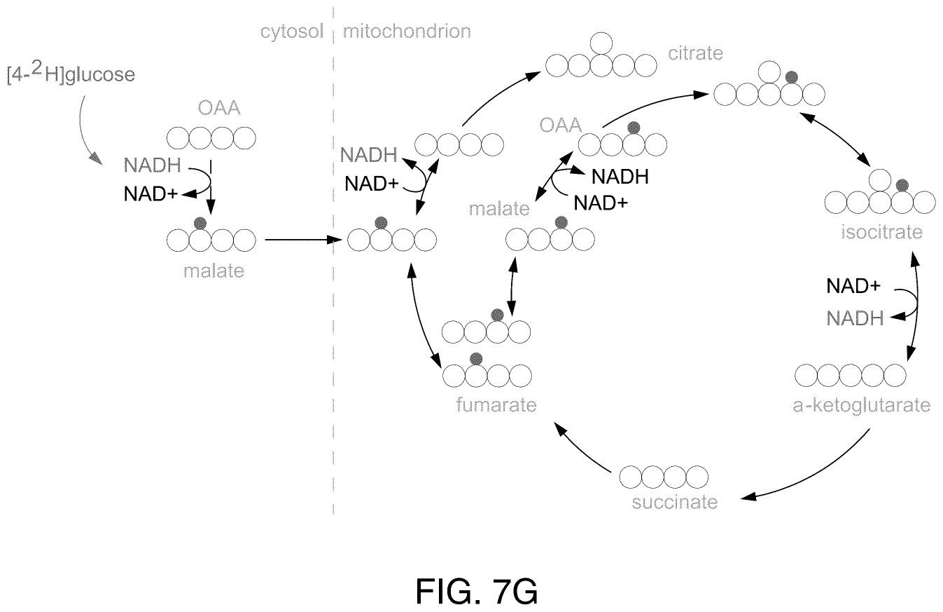

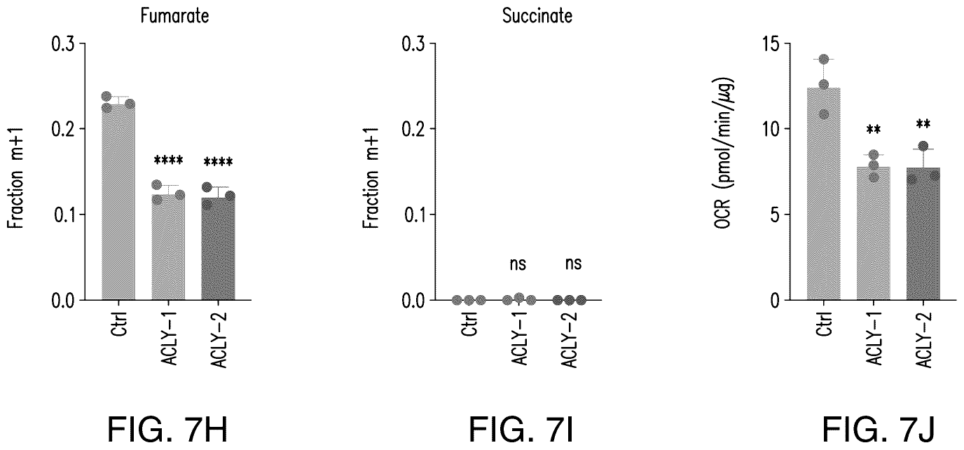

To determine whether an ACL-mediated TCA cycle exists in non-transformed cells, the present disclosure next traced the fate of uniformly labeled ¹³C-glucose in mouse embryonic stem cells (ESCs), which are rapidly proliferating primary cells capable of indefinite self-renewal when cultured in the presence of serum and leukemia inhibitory factor (LIF). Similar to NSCLC lines, ESCs show a strong decrease in the enrichment of the m+2 isotopologue of malate relative to citrate, and this disconnect is mitigated by treatment with an ACL inhibitor (FIGS. 2A and 2B). To compare the effects of ACL inhibition with disruption of the canonical TCA cycle, the present disclosure used CRISPR/Cas9 editing to generate clonal ESC lines with genetic disruption of Acly or Aco2 (FIGS. 7A and 7B). Acly disruption had no consistent effect on m+2 enrichment in citrate but consistently increased m+2 enrichment in downstream TCA cycle metabolites (fumarate, malate, aspartate) by over two-fold, resulting in significant elevation of the mal+2/cit+2 ratio in Acly-edited lines (FIGS. 2C and 2D). Conversely, Aco2 disruption reduced m+2 enrichment of downstream TCA cycle metabolites and decreased the mal+2/cit+2 ratio (FIG. 7C). Surprisingly, despite ACO2's role as a canonical TCA cycle enzyme, Aco2 disruption minimally affected steady-state levels of TCA cycle metabolites (FIG. 7D). In contrast, Acly mutation dramatically altered levels of TCA cycle metabolites specifically associated with cytosolic citrate processing (citrate, malate, aspartate, and fumarate), but not canonical TCA cycle metabolism (succinate, a-ketoglutarate) (FIGS. 1B and 2E).

The effect of Acly mutation on steady-state levels of TCA cycle intermediates is consistent with a model wherein a considerable portion of the TCA cycle flows through ACL. To test the hypothesis that ACL anchors a non-canonical TCA cycle, the present disclosure directly assessed the degree to which ACL mediates citrate recycling. Cytosolic processing of citrate to malate requires hydride donation from NADH, which can be traced by culturing cells with [4-²H] glucose to label up to half of cytosolic NADH pools (FIG. 2F)²². Acly mutation had no apparent effect on the ability of [4-²H] glucose to label NADH pools: both control and edited cells exhibit similar fractional enrichment of NADH and lactate, which becomes labeled when cytosolic pyruvate is reduced by lactate dehydrogenase (FIGS. 7E and 7F). Acly-edited clones did, however, display significantly lower m+1 enrichment of malate, suggesting that wild-type cells generate a portion of malate from the reduction of cytosolic oxaloacetate in an ACL-dependent manner (FIG. 2G). Critically, Acly mutation more than halved the fraction of citrate containing a deuterium label, indicating that cytosolic malate generated downstream of ACL is indeed recycled back into the mitochondria to drive citrate regeneration, and that this process is impaired in the absence of ACL (FIGS. 2H and 7G-7I). Consistent with impaired transfer of cytosolic reducing equivalents to the mitochondria, Acly mutation increased the cytosolic NADH/NAD+ratio, measured as the ratio of lactate/pyruvate²³, and decreased mitochondrial oxygen consumption, (FIGS. 2I and 7J). Together, these results demonstrate that a significant portion of the TCA cycle transits through the cytoplasm in an ACL-dependent manner as an alternative to the traditional TCA cycle.

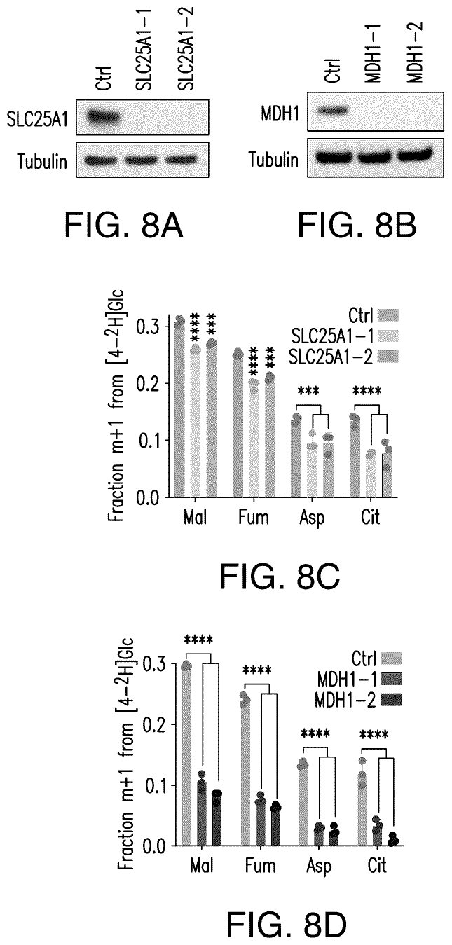

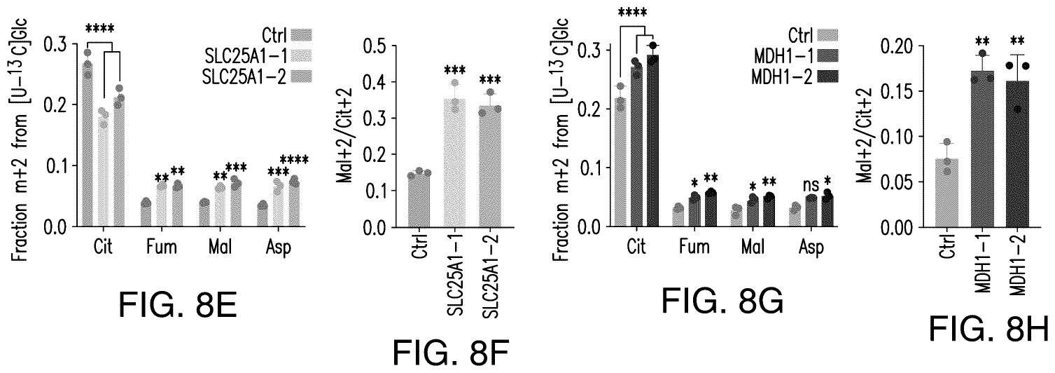

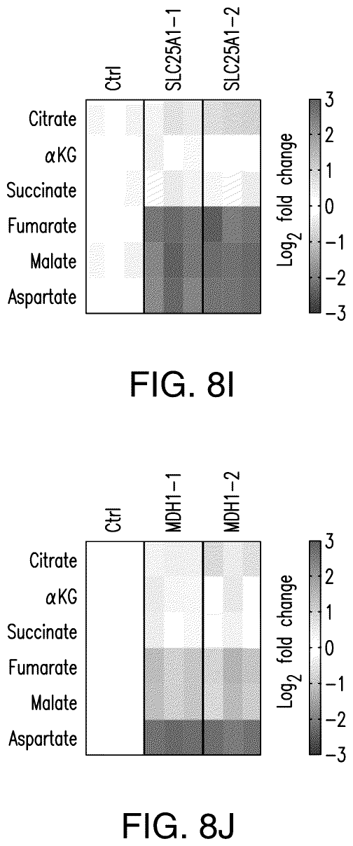

To further test whether SLC25A1, ACL and MDH1 form a non-canonical TCA cycle, the present disclosure generated clonal ESC lines deficient for SLC25A1 and MDH1 (FIGS. 8A and 8B). Deuterated glucose tracing revealed that, like ACL, both SLC25A1 and MDH1 were required for citrate regeneration from cytosolic oxaloacetate (FIGS. 8C and 8D). Accordingly, Slc25a1-and Mdh1-edited cells exhibited ¹³C-glucose labeling patterns consistent with reduced non-canonical TCA cycle activity: Slc25a1 and Mdh1 editing increased enrichment of m+2 isotopologues in TCA cycle metabolites downstream of citrate and elevated the mal+2/cit+2 ratio (FIGS. 8E-8H). Furthermore, Slc25a1 and Mdh1 mutation, like Acly mutation, reduced levels of TCA cycle metabolites malate and fumarate that are downstream of cytosolic citrate processing (FIGS. 8I and 8J). SLC25A1 loss also reduced aspartate pools, but MDH1 loss did not, consistent with its role in the malate-aspartate shuttle that consumes cytosolic aspartate²⁴. These data collectively demonstrate that SLC25A1, ACL and MDH1 coordinate a cross-compartment cycle of citrate metabolism.

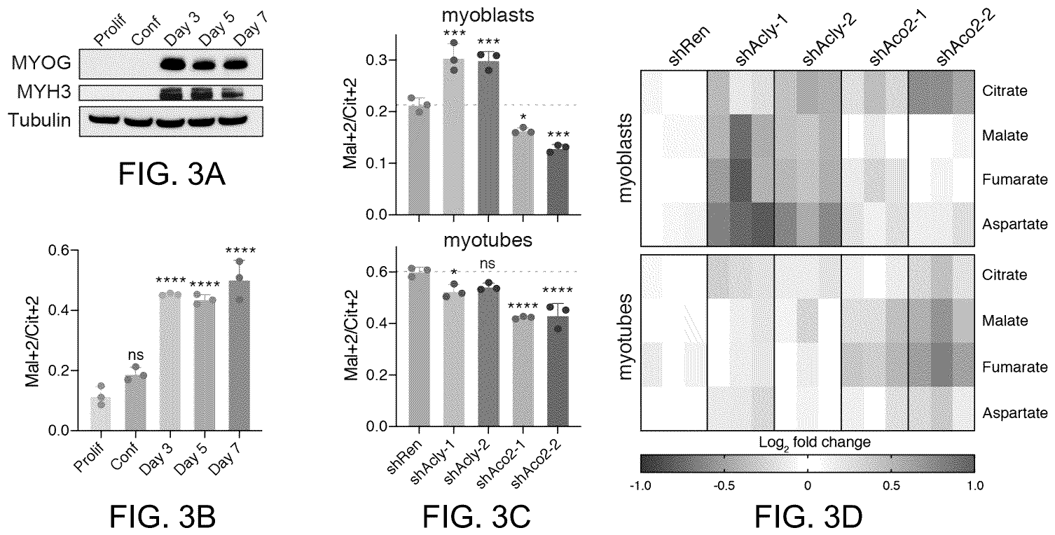

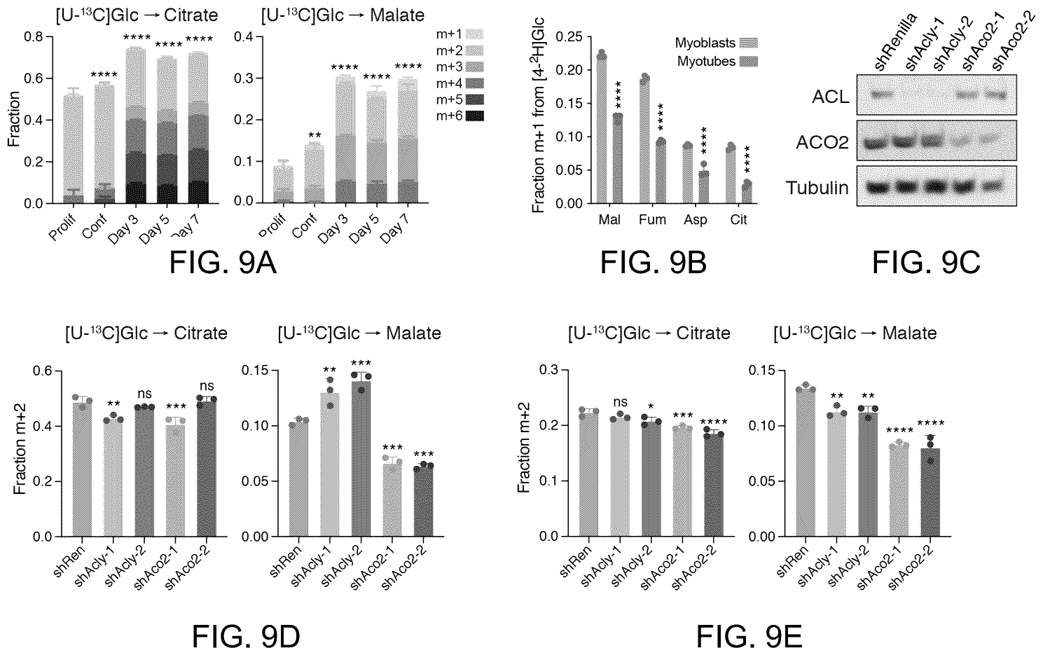

Sir Hans Krebs originally elucidated the canonical TCA cycle in pigeon breast muscle^{25,26}. The present disclosure therefore asked whether different cell types harbor different preferences for canonical versus non-canonical TCA cycle metabolism. To test this idea, the present disclosure cultured C2C12 mouse myoblasts, which can be differentiated in vitro into mature myotubes (FIG. 3A). Myogenic differentiation was accompanied by enhanced glucose incorporation into TCA cycle metabolites and increased enrichment of higher order isotopologues indicative of successive turns of the TCA cycle (FIG. 9A). Consequently, differentiated myotubes cultured with uniformly ¹³C-labeled glucose exhibited a mal+2/cit+2 ratio over five-fold higher than proliferating myoblasts, reflecting elevated glucose oxidation through the canonical TCA cycle (FIG. 3B). Consistently, [4-²H] glucose tracing revealed diminished production of malate, fumarate and citrate from cytosolic oxaloacetate in myotubes compared with myoblasts (FIG. 9B).

To determine whether myoblasts indeed carried out more non-canonical TCA cycle activity than their differentiated counterparts, the present disclosure engineered cells to express doxycycline-inducible hairpins targeting Acly or Aco2 (FIG. 9C). Similar to results observed in cancer cells and ESCs, ACL inhibition in myoblasts significantly increased the mal+2/cit+2 ratio (FIG. 3C, top and FIG. 9D). In contrast, ACL inhibition had no notable effect on the mal+2/cit+2 ratio in differentiated myotubes, suggesting that the conversion of citrate to malate is largely ACL-independent in myotubes (FIG. 3C, bottom, and FIG. 9E). Conversely, while ACO2 inhibition decreased the mal+2/cit+2 ratio in myoblasts, this effect was larger in myotubes, consistent with enhanced citrate processing through the canonical TCA cycle following differentiation (FIG. 3C). Accordingly, myotubes and myoblasts exhibited different requirements for ACO2 and ACL to maintain pools of TCA cycle metabolites. As in ESCs, ACL inhibition significantly increased citrate pools and decreased levels of fumarate, malate and aspartate, and this effect was stronger in myoblasts than in myotubes (FIG. 3D). In contrast, ACO2 inhibition had little effect on levels of TCA cycle metabolites in myoblasts and surprisingly tended to increase metabolite levels in myotubes, indicating that ACO2 loss causes greater metabolic disruption in myotubes relative to myoblasts (FIG. 3D). Collectively, these results indicate that the degree to which cells employ the canonical TCA cycle is at least partially determined by cell state.

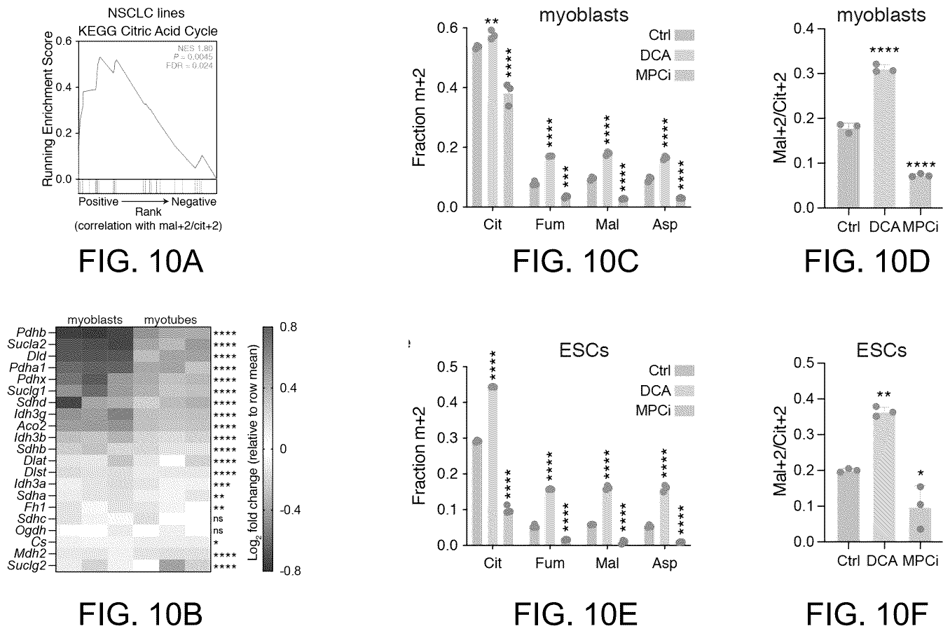

Many variables, including gene expression, post-translational modifications, substrate availability and allosteric modulators contribute to regulating flux through the TCA cycle²⁷. Comparing gene expression with TCA cycle activity in NSCLC lines revealed that genes positively correlated with the mal+2/cit+2 ratio were highly enriched for TCA cycle genes (FIG. 10A). Similarly, most TCA cycle genes were significantly induced upon myogenic differentiation, indicating that coordinated transcriptional upregulation of TCA cycle genes is a common feature of cells engaging the canonical TCA cycle (FIG. 10B). In particular, the present disclosure noted that all subunits of the pyruvate dehydrogenase complex (PDHC) that initiates carbon entry into the TCA cycle were highly upregulated upon myogenic differentiation (FIG. 10B). The present disclosure therefore tested whether increasing pyruvate entry into the TCA cycle enhanced canonical TCA cycle metabolism. Indeed, both myoblasts and ESCs treated with dichloroacetate to potentiate PDHC activity²⁸ increased incorporation of glucose-derived carbons not only into citrate but also into downstream metabolites, resulting in an elevated mal+2/cit+2 ratio consistent with enhanced canonical TCA cycle activity (FIGS. 10C-10F). Reciprocally, pharmacological inhibition of the mitochondrial pyruvate carrier reduced incorporation of glucose-derived carbons into TCA cycle metabolites and repressed the mal+2/cit+2 ratio (FIGS. 10C-10F). Thus, TCA cycle choice is at least partly determined by the amount of pyruvate captured for oxidation by PDHC.

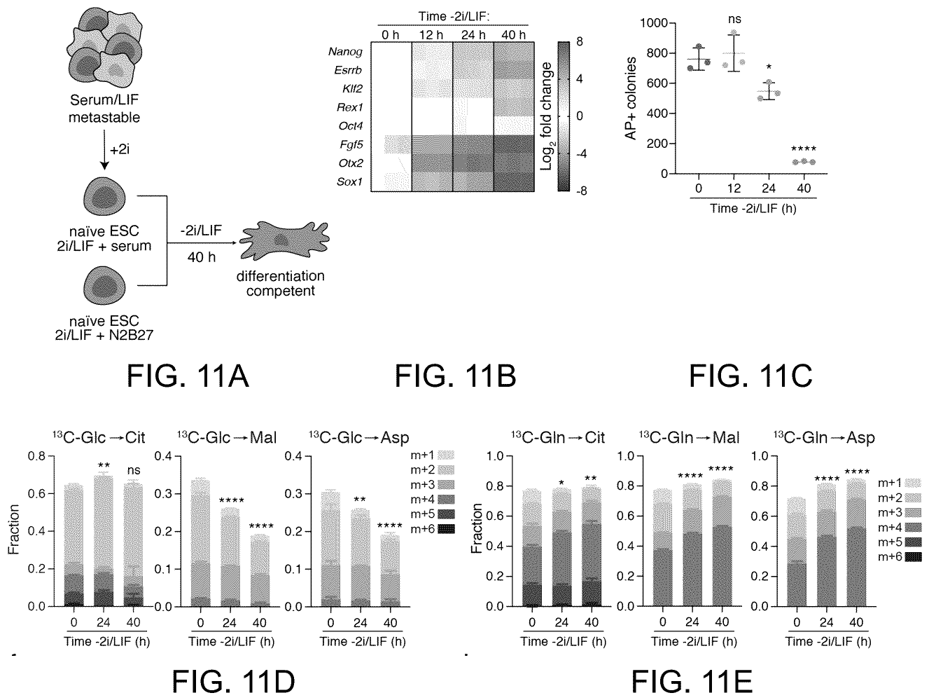

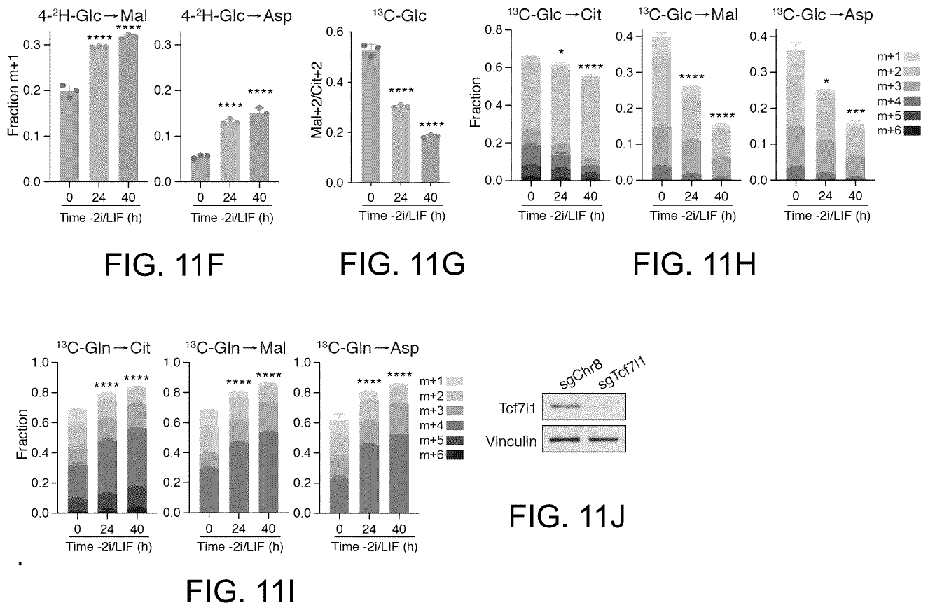

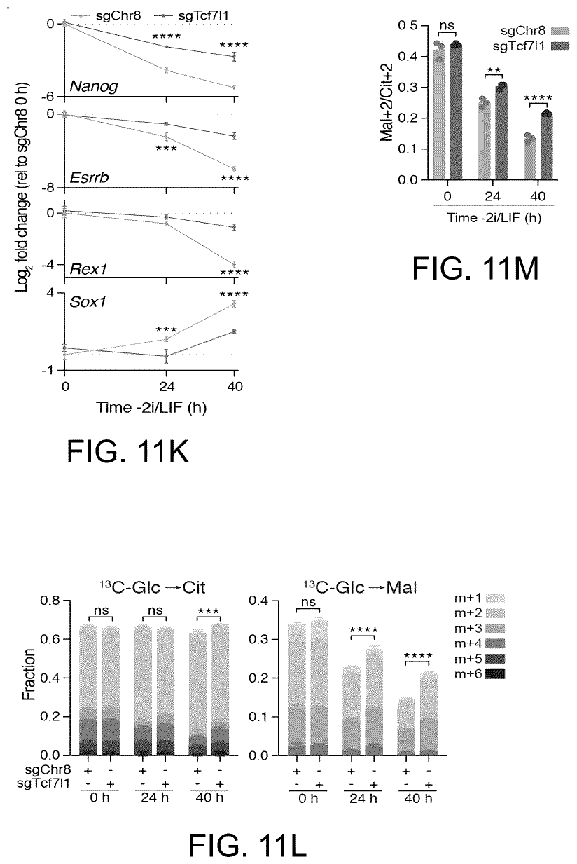

Based on the present disclosure observation that TCA cycle engagement is cell state dependent, the present disclosure next asked whether the mode of TCA cycle engagement contributes to the metabolic diversity observed in cultured cells. Stem cell commitment and differentiation are frequently marked by metabolic reprogramming and changes in TCA cycle substrate oxidation⁸⁻¹⁰. The present disclosure used ESCs as a well-defined model system for self-renewal and early lineage commitment, as many studies demonstrate that fate changes in ESCs are accompanied by notable changes in TCA cycle activity²⁹⁻³³. ESCs grown in the presence of LIF and inhibitors against GSK3β and MEK (2i) represent the naïve ground state of pluripotency reminiscent of the pre-implantation epiblast; withdrawal of these factors (−2i/LIF) allows cells to exit the naïve pluripotent state and gain differentiation competence (FIGS. 11A-11F) 34.35. Supplementing serum/LIF-cultured ESCs with 2i confirmed that ESCs in the naïve, ground state of pluripotency use both glucose and glutamine to generate TCA cycle intermediates (FIGS. 11D and 11E) as previously reported^{29,30}. Loss of naïve pluripotency triggers major changes in TCA cycle dynamics. Specifically, cells induced to exit the naïve ground state notably increase incorporation of glutamine-derived carbon (FIG. 11E), consistent with previous report demonstrating that more committed cells exhibit enhanced reliance upon exogenous glutamine³⁶.

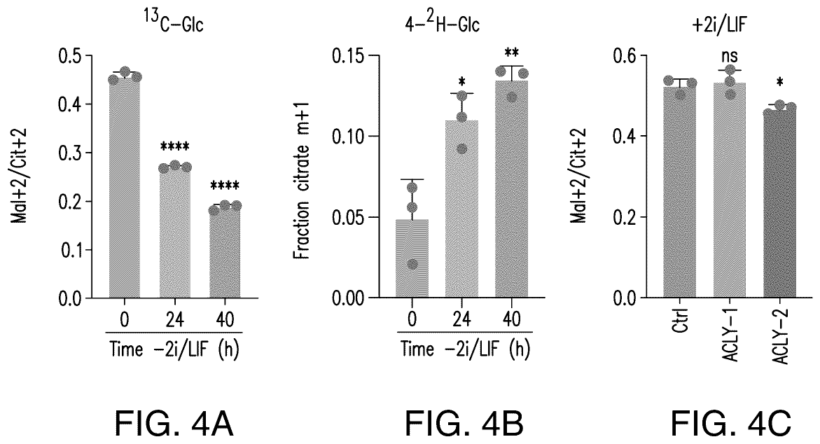

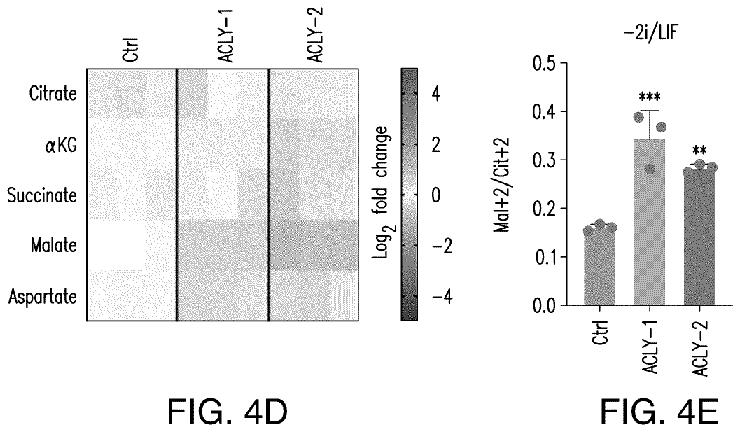

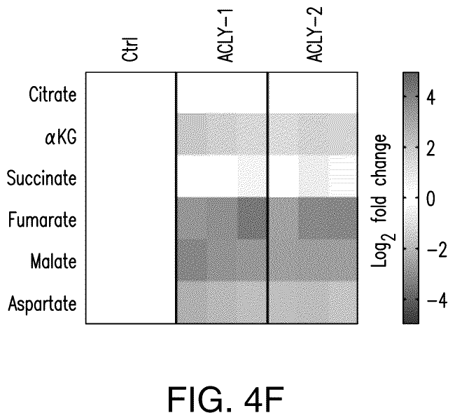

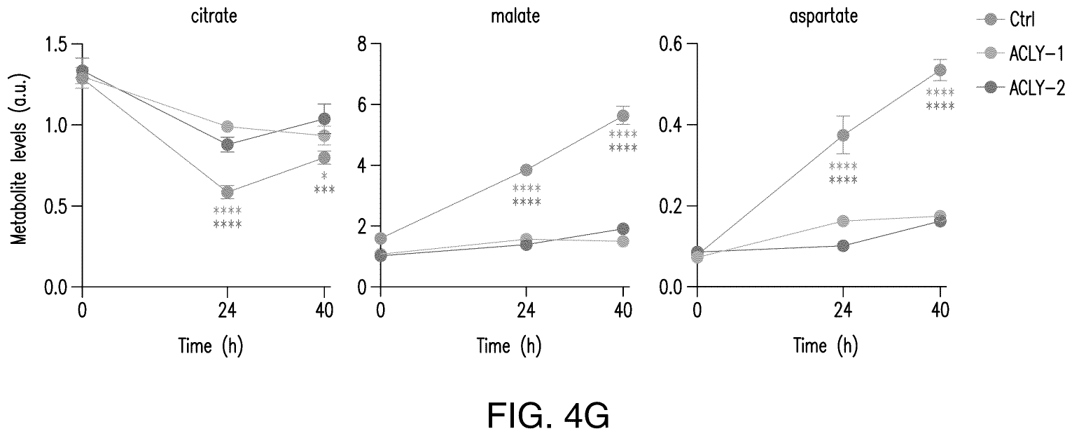

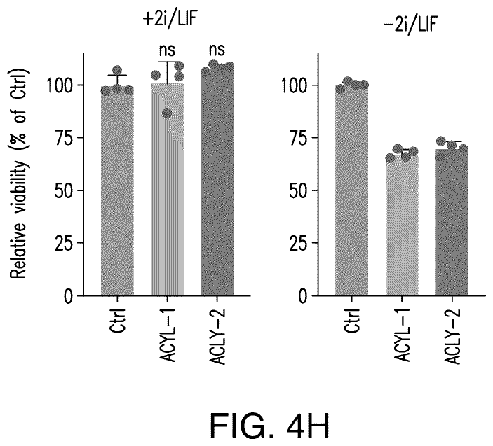

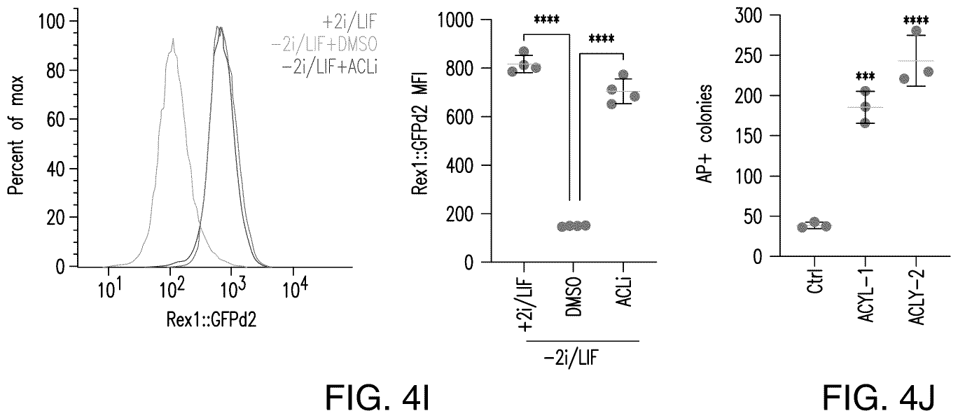

Analysis of glucose isotopologue labeling patterns revealed that naïve ESCs have a high mal+2/cit+2 ratio that is progressively lost during exit from naïve pluripotency alongside a concomitant increase in production of citrate from cytosolic intermediates (FIGS. 4A, 4B, and 11F). This metabolic shift was not due to changes in culture medium as ESCs cultured serum-free in 2i/LIF also repressed glucose utilization and decreased the mal+2/cit+2 ratio following 2i/LIF withdrawal despite continuous culture in the same serum-free medium formulation (FIGS. 11G-11I). Furthermore, cells deficient for Tcf3/Tcf7l1, a repressor of the naïve pluripotency gene network whose loss impedes exit from naïve pluripotency^{35,37}, exhibited dampened metabolic reprogramming corresponding to their delayed exit from the naïve pluripotent state (FIGS. 11J-11M).

Together, these results indicate that ESCs switch from canonical to non-canonical TCA cycle activity as they exit the naïve pluripotent state. Indeed, ACL loss had little effect on TCA cycle dynamics in naïve ESCs: Acly-edited clones had no change in the mal+2/cit+2 ratio and only minimal differences in steady-state levels of TCA cycle metabolites (FIGS. 4C and 4D). In contrast, ACL loss doubled the mal+2/cit+2 ratio and triggered large (up to 7-fold) changes in TCA cycle metabolite levels in cells grown-2i/LIF (FIGS. 4E and 4F). Collectively, these data support a model wherein ESCs increase reliance on ACL-mediated non-canonical TCA cycle activity as they lose pluripotency and acquire differentiation competence.

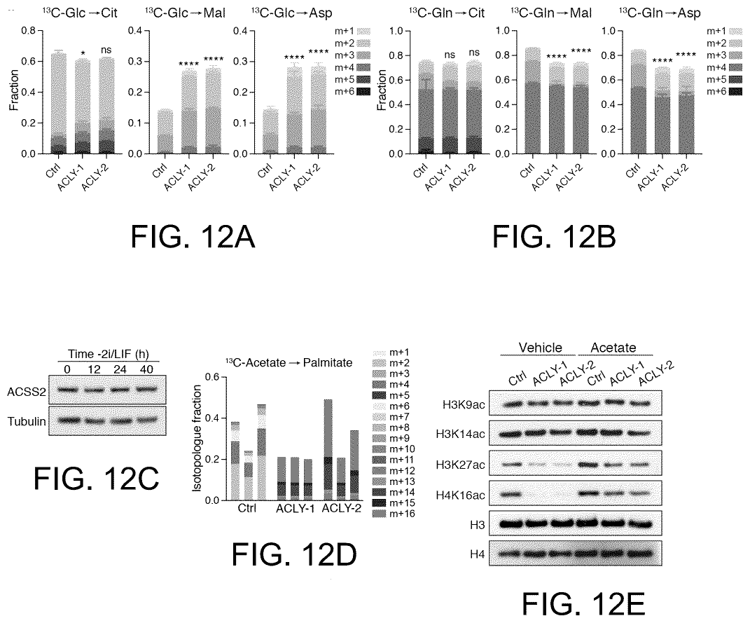

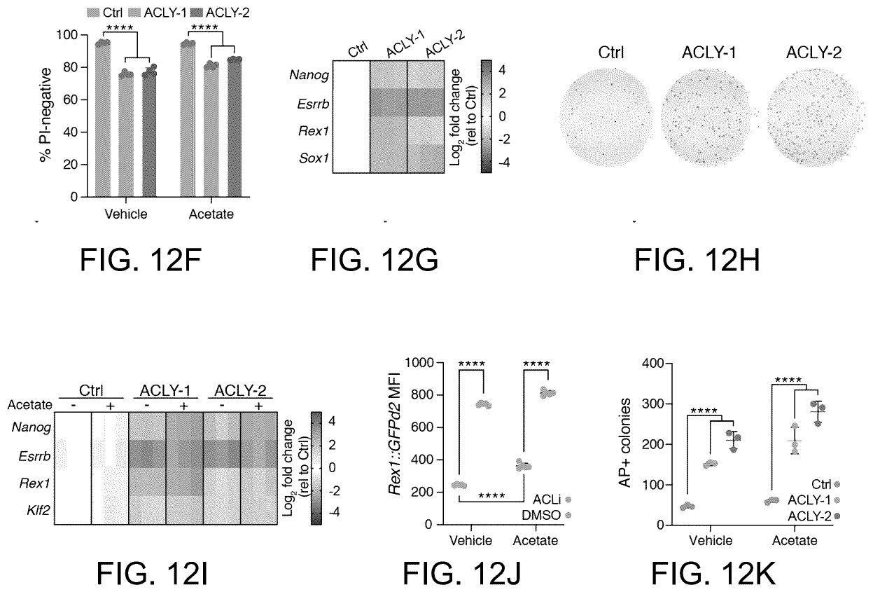

These data raise the possibility that variable use of TCA cycle pathways contributes to the establishment of metabolic identity. To test this hypothesis, the present disclosure monitored changes in TCA cycle metabolite levels in control and Acly-edited clones during exit from pluripotency. Strikingly, exit from pluripotency induced large changes in TCA cycle metabolite levels that were blunted by ACL loss (FIG. 4G). Furthermore, Acly-edited clones maintained higher incorporation of glucose-derived carbons and lower incorporation of glutamine-derived carbons than their control counterparts, indicating that ACL is required for the metabolic shifts that occur during exit from pluripotency (FIGS. 12A and 12B).

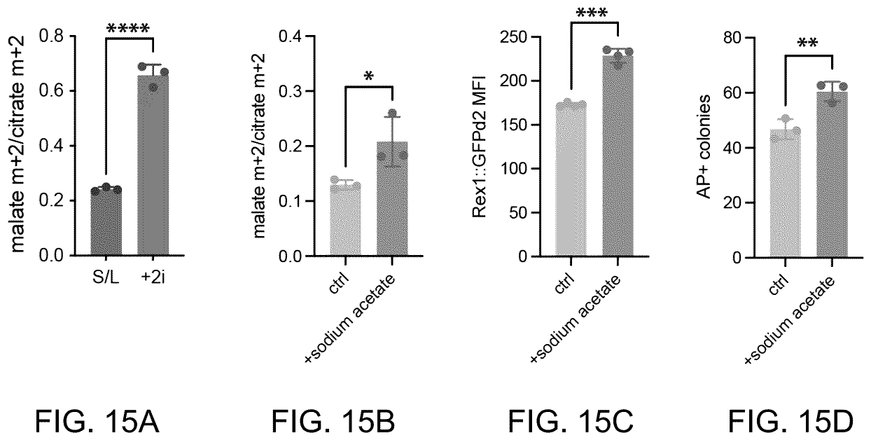

The failure of Acly-edited clones to establish appropriate metabolic identity had functional consequences. Cells without ACL exhibited impaired viability specifically upon exit from naïve pluripotency (FIG. 4H). This impaired viability was not due simply to deficient cytosolic acetyl-CoA following ACL loss: exogenous acetate restored histone acetylation and contributed to de novo lipid synthesis-two processes dependent upon cytosolic acetyl-CoA19—but only minimally rescued the decline in viability observed in Acly-edited clones (FIGS. 12C-12F). These results demonstrate that upon exit from naïve pluripotency, cells rely on the non-canonical TCA cycle to maintain TCA cycle intermediates and cell viability. To further test the idea that ACL loss spares naïve pluripotent cells and specifically affects cells that have acquired differentiation competence, the present disclosure assessed the effect of ACL loss on expression of naïve pluripotency markers following 2i/LIF withdrawal. Using ESCs harboring a reporter of naïve pluripotency (Rex1::GFPd2)³⁸, the present disclosure found that the expected downregulation of reporter activity following 2i/LIF withdrawal³⁴ was almost completely prevented by ACL inhibition (FIG. 4I). Similarly, Acly-edited clones subjected to 2i/LIF withdrawal demonstrated increased expression of naïve pluripotency genes Nanog, Esrrb and Rex1, impaired induction of the differentiation marker Sox1, and enhanced ability to form alkaline phosphatase positive (AP+) colonies when reseeded into medium containing 2i/LIF (FIGS. 4J, 12G, and 12H). None of these phenotypes were reversed by exogenous acetate: rather, exogenous acetate tended to increase markers of naïve pluripotency and colony formation ability (FIGS. 12I-12K) consistent with previous reports that acetate promotes ESC self-renewal³⁹. Collectively, these results demonstrate that upon exit from naïve pluripotency ACL loss disrupts TCA cycle dynamics and provides a selective disadvantage that is not the result of impaired generation of cytosolic acetyl-CoA. The present disclosure further showed that acetate supplementation enhances processivity in mouse embryonic stem cells (FIGS. 15A-15D).

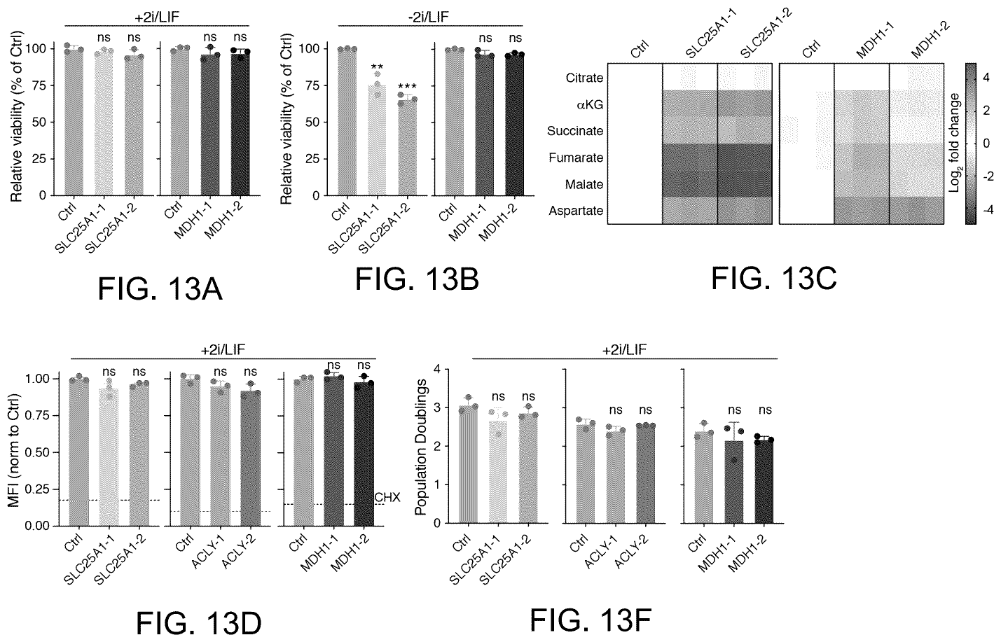

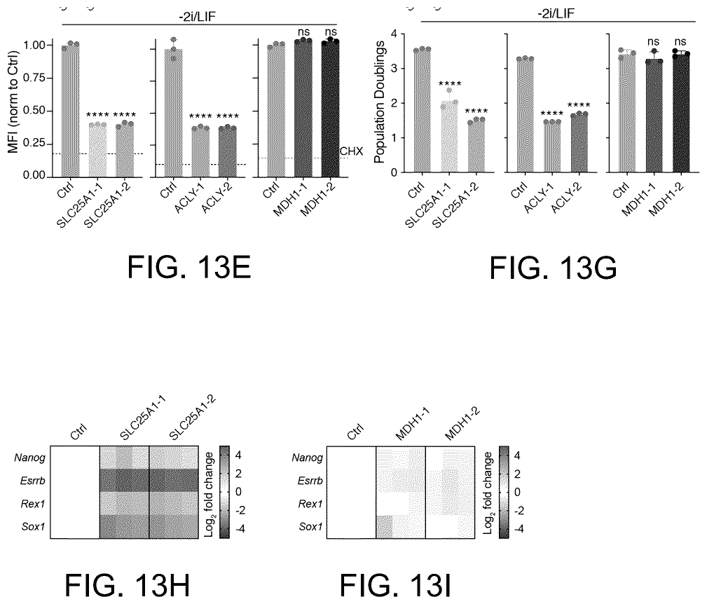

The present disclosure therefore next tested whether other components of the non-canonical TCA cycle, SLC25A1 and MDH1, were likewise selectively engaged during exit from naïve pluripotency. Like ACL, neither SLC25A1 nor MDH1 was required for viability of naïve pluripotent ESCs (FIG. 13A). Moreover, like ACL, SLC25A1 loss specifically impaired viability of cells exiting the naïve pluripotent state (FIG. 13B). In contrast, MDH1 was dispensable for viability during exit from naïve pluripotency (FIG. 13B). Notably, while ACL, SLC25A1 and MDH1 were all required to sustain TCA cycle metabolites fumarate and malate during exit from naïve pluripotency, only SLC25A1 and ACL were required to maintain aspartate pools (FIG. 13E). Aspartate, which contributes to protein and nucleotide biosynthesis, is a critical output of the TCA cycle in proliferating cells^{24,40}. Consistently, when induced to exit naïve pluripotency, both Acly-and Slc25a1-edited cells had impaired protein translation and reduced proliferation, whereas Mdh1-edited cells did not (FIGS. 13D-13G). Accordingly, while Acly-and Slc25a1-edited cells preserved naïve pluripotency gene signatures, this effect was blunted in Mdh1-edited cells (FIGS. 13H and 13I). Together, these results demonstrate that SLC25A1, ACL and MDH1 contribute to the establishment of metabolic identity as cells exit the naïve pluripotent state, and cells that are unable to activate ACL-dependent non-canonical TCA cycle metabolism exhibit compromised viability.

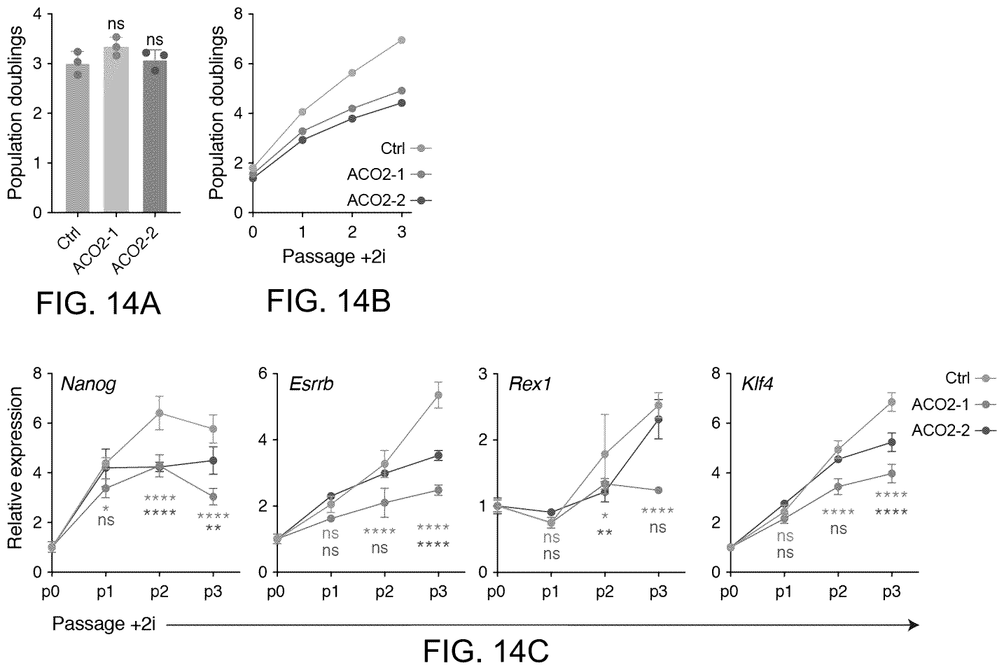

To further test the model that appropriate TCA cycle engagement underlies establishment of cell identity, the present disclosure tested whether efficient induction of the naïve, ground state of pluripotency required canonical TCA cycle metabolism. Aco2 disruption had no effect on proliferation in cells cultured in serum/LIF, which exhibit significant ACL-mediated non-canonical TCA cycle activity (FIGS. 2A-2I and 14A). In contrast, supplementing 2i to initiate conversion into the naïve, ground state of pluripotency specifically slowed proliferation of Aco2-edited clones, but not their control counterparts (FIG. 14B). Consistent with impairing transition to the naïve, ground state of pluripotency, Aco2 loss delayed induction of naïve pluripotency markers upon conversion to medium containing 2i (FIG. 14C). Collectively, these results underscore the role of TCA cycle configuration in facilitating cell state transitions.

Here, the present disclosure identifies a non-canonical TCA cycle active in both normal and transformed cells. The possibility of a similar citrate-malate shuttle has been proposed in the past but never directly demonstrated^{16,41}. A disconnect between glucose labeling of citrate and downstream TCA cycle metabolites was also observed in activated natural killer cells⁴², and cytosolic NAD⁺ regeneration enabled by the conversion of citrate to malate was linked to maintenance of cytosolic redox in ethanol-perfused rat livers⁴³. These observations raise the possibility that a wide array of mammalian cell types engage in citrate-malate shuttling, but whether this shuttling constitutes a true cycle in which oxaloacetate produced from cytosolic citrate is indeed recycled in the mitochondrion, and whether this recycling contributes to the disconnect in glucose labeling between TCA cycle intermediates, remained unknown. By combining isotope tracing with genetic manipulation of ACL, SLC25A1 and MDH1, the present disclosure provides direct evidence that the proposed citrate-malate shuttle indeed represents a bona fide cycle with differential activity across mammalian cell states.

There are numerous potential advantages to engaging in this alternative to the canonical TCA cycle. While the canonical TCA cycle results in loss of two molecules of reduced carbon as CO₂ at each turn, citrate metabolism via ACL enables cells to retain reduced carbon for biosynthesis, providing cytosolic acetyl-CoA for protein acetylation and lipid synthesis. Furthermore, because production of pyruvate through glycolysis reduces an oxidized NAD⁺, each molecule of pyruvate metabolized in the mitochondrion causes a redox imbalance in the cytosol. This problem is solved by the non-canonical TCA cycle, which generates oxidized NAD⁺ through the reduction of cytosolic oxaloacetate to malate. Finally, increasing evidence suggests that overproduction of mitochondrial NADH relative to ATP demand is a restraint on cell proliferation⁴⁴. By circumventing several steps of the canonical mitochondrial TCA cycle, the non-canonical TCA cycle may relieve cells of this reductive challenge while sustaining oxaloacetate production for citrate regeneration.

The present disclosure further demonstrates that the method of TCA cycle engagement is a determinant of cellular identity. The present disclosure find that cancer cells and stem cells exhibit significant heterogeneity in the degree to which they employ the non-canonical TCA cycle and that the switch from canonical to non-canonical TCA cycle metabolism is a required component of the metabolic rewiring that occurs during exit from pluripotency. Consequently, ESCs that cannot switch their mode of TCA cycle metabolism are blocked from exiting the naïve ground state of pluripotency, indicating that changes in TCA cycle configuration are required for changes in cell fate. While the intra-or extracellular drivers of TCA cycle choice remain to be fully elucidated, the present disclosure suggests that the mode of TCA cycle metabolism contributes to the metabolic diversity observed in cultured cells. Notably, in contrast to their in vitro counterparts, cancer cells growing in vivo exhibit little loss of glucose label between citrate and malate^{6,7}, suggesting that differential engagement of the canonical TCA cycle may contribute to the observed disconnect between in vitro and in vivo metabolic phenotypes. Intriguingly, while a recent study demonstrated overall similarity in metabolic gene essentiality in pancreatic cancer cells cultured in vitro or engrafted in vivo, cancer cells were more reliant on Acly in vitro and were conversely more dependent upon Aco2 in vivo⁴⁵. Collectively, these studies underscore the diversity of metabolic strategies that support cellular bioenergetics and reveal that TCA cycle behavior is dynamic and entwined with cell state.

Although the presently disclosed subject matter and its advantages have been described in detail, it should be understood that various changes, substitutions and alterations can be made herein without departing from the spirit and scope of the invention. Moreover, the scope of the present application is not intended to be limited to the particular embodiments of the process, machine, manufacture, and composition of matter, means, methods and steps described in the specification. As one of ordinary skill in the art will readily appreciate from the invention of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Various patents, patent applications, publications, product descriptions, protocols, and sequence accession numbers are cited throughout this application, the inventions of which are incorporated herein by reference in their entireties for all purposes.

### 7.4 REFERENCES

1 DeBerardinis, R. J. & Chandel, N. S. Fundamentals of cancer metabolism. Sci Adv 2, c1600200, doi: 10.1126/sciadv.1600200 (2016).

2 Kim, J. & DeBerardinis, R. J. Mechanisms and Implications of Metabolic Heterogeneity in Cancer. Cell metabolism 30, 434-446, doi: 10.1016/j.cmet.2019.08.013 (2019).

3 Muir, A., Danai, L. V. & Vander Heiden, M. G. Microenvironmental regulation of cancer cell metabolism: implications for experimental design and translational studies. Dis Model Mech 11, doi: 10.1242/dmm.035758 (2018).

4 Faubert, B., Solmonson, A. & DeBerardinis, R. J. Metabolic reprogramming and cancer progression. Science 368, doi: 10.1126/science.aaw5473 (2020).

5 Hui, S. et al. Glucose feeds the TCA cycle via circulating lactate. Nature 551, 115-118, doi: 10.1038/nature24057 (2017).

6 Davidson, S. M. et al. Environment Impacts the Metabolic Dependencies of Ras-Driven Non-Small Cell Lung Cancer. Cell metabolism 23, 517-528, doi: 10.1016/j.cmet.2016.01.007 (2016).

7 Marin-Valencia, I. et al. Analysis of tumor metabolism reveals mitochondrial glucose oxidation in genetically diverse human glioblastomas in the mouse brain in vivo. Cell metabolism 15, 827-837, doi: 10.1016/j.cmet.2012.05.001 (2012).

8 Shapira, S. N. & Christofk, H. R. Metabolic Regulation of Tissue Stem Cells. Trends Cell Biol 30, 566-576, doi: 10.1016/j.tcb.2020.04.004 (2020).

9 Intlekofer, A. M. & Finley, L. W. S. Metabolic signatures of cancer cells and stem cells. Nature Metabolism 1, 177-188, doi: 10.1038/s42255-019-0032-0 (2019).

10 Wei, P., Dove, K. K., Bensard, C., Schell, J. C. & Rutter, J. The Force Is Strong with This One: Metabolism (Over) powers Stem Cell Fate. Trends Cell Biol 28, 551-559, doi: 10.1016/j.tcb.2018.02.007 (2018).

11 Wang, T. et al. Gene Essentiality Profiling Reveals Gene Networks and Synthetic Lethal Interactions with Oncogenic Ras. Cell 168, 890-903 e815, doi: 10.1016/j.cell.2017.01.013 (2017).

12 Wainberg, M. et al. A genome-wide almanac of co-essential modules assigns function to uncharacterized genes. bioRxiv, 827071, doi: 10.1101/827071 (2019).

13 DeBerardinis, R. J. et al. Beyond acrobic glycolysis: transformed cells can engage in glutamine metabolism that exceeds the requirement for protein and nucleotide synthesis. Proceedings of the National Academy of Sciences of the United States of America 104, 19345-19350, doi: 10.1073/pnas.0709747104 (2007).

14 Bayraktar, E. C. et al. Metabolic coessentiality mapping identifies C12orf49 as a regulator of SREBP processing and cholesterol metabolism. Nat Metab 2, 487-498, doi: 10.1038/s42255-020-0206-9 (2020).

15 Tsherniak, A. et al. Defining a Cancer Dependency Map. Cell 170, 564-576 c516, doi: 10.1016/j.cell.2017.06.010 (2017).

16 Hatzivassiliou, G. et al. ATP citrate lyase inhibition can suppress tumor cell growth. Cancer cell 8, 311-321, doi: 10.1016/j.ccr.2005.09.008 (2005).

17 Bauer, D. E., Hatzivassiliou, G., Zhao, F., Andreadis, C. & Thompson, C. B. ATP citrate lyase is an important component of cell growth and transformation. Oncogene 24, 6314-6322, doi: 10.1038/sj.onc.1208773 (2005).

18 Chen, P. H. et al. Metabolic Diversity in Human Non-Small Cell Lung Cancer Cells. Molecular cell 76, 838-851 e835, doi: 10.1016/j.molcel.2019.08.028 (2019).

19 Zhao, S. et al. ATP-Citrate Lyase Controls a Glucose-to-Acetate Metabolic Switch. Cell Rep 17, 1037-1052, doi: 10.1016/j.celrep.2016.09.069 (2016).

20 Alves, T. C. et al. Integrated, Step-Wise, Mass-Isotopomeric Flux Analysis of the TCA Cycle. Cell metabolism 22, 936-947, doi: 10.1016/j.cmet.2015.08.021 (2015).

21 Sullivan, L. B. et al. Aspartate is an endogenous metabolic limitation for tumour growth. Nat Cell Biol 20, 782-788, doi: 10.1038/s41556-018-0125-0 (2018).

22 Lewis, C. A. et al. Tracing compartmentalized NADPH metabolism in the cytosol and mitochondria of mammalian cells. Molecular cell 55, 253-263, doi: 10.1016/j.molcel.2014.05.008 (2014).

23 Williamson, D. H., Lund, P. & Krebs, H. A. The redox state of free nicotinamide-adenine dinucleotide in the cytoplasm and mitochondria of rat liver. The Biochemical journal 103, 514-527, doi: 10.1042/bj1030514 (1967).

24 Birsoy, K. et al. An Essential Role of the Mitochondrial Electron Transport Chain in Cell Proliferation Is to Enable Aspartate Synthesis. Cell 162, 540-551, doi: 10.1016/j.cell.2015.07.016 (2015).

25 Krebs, H. A. & Eggleston, L. V. The oxidation of pyruvate in pigeon breast muscle. The Biochemical journal 34, 442-459, doi: 10.1042/bj0340442 (1940).

26 Krebs, H. A. & Johnson, W. A. The role of citric acid in intermediate metabolism in animal tissues. FEBS Lett 117 Suppl, K1-10, doi: 10.4159/harvard.9780674366701.c143 (1980).

27 Berg, J. M., Tymoczko, J. L., Stryer, L. & Stryer, L. Biochemistry. 5th edn, (W. H. Freeman., 2002).

28 Stacpoole, P. W. The pharmacology of dichloroacetate. Metabolism 38, 1124-1144, doi: 10.1016/0026-0495 (89) 90051-6 (1989).

29 Carey, B. W., Finley, L. W., Cross, J. R., Allis, C. D. & Thompson, C. B. Intracellular alpha-ketoglutarate maintains the pluripotency of embryonic stem cells. Nature 518, 413-416, doi: 10.1038/nature13981 (2015).

30 Zhang, J. et al. LIN28 Regulates Stem Cell Metabolism and Conversion to Primed Pluripotency. Cell stem cell 19, 66-80, doi: 10.1016/j.stem.2016.05.009 (2016).

31 Zhou, W. et al. HIF1alpha induced switch from bivalent to exclusively glycolytic metabolism during ESC-to-EpiSC/hESC transition. EMBO J 31, 2103-2116, doi: 10.1038/emboj.2012.71 (2012).

32 Gu, W. et al. Glycolytic Metabolism Plays a Functional Role in Regulating Human Pluripotent Stem Cell State. Cell stem cell 19, 476-490, doi: 10.1016/j.stem.2016.08.008 (2016).

33 Zhang, J. et al. UCP2 regulates energy metabolism and differentiation potential of human pluripotent stem cells. EMBO J 30, 4860-4873, doi: 10.1038/emboj.2011.401 (2011).

34 Kalkan, T. et al. Tracking the embryonic stem cell transition from ground state pluripotency. Development 144, 1221-1234, doi: 10.1242/dev.142711 (2017).

35 Betschinger, J. et al. Exit from pluripotency is gated by intracellular redistribution of the bHLH transcription factor Tfe3. Cell 153, 335-347, doi: 10.1016/j.cell.2013.03.012 (2013).

36 Vardhana, S. A. et al. Glutamine independence is a selectable feature of pluripotent stem cells. Nature Metabolism 1, 676-687, doi: 10.1038/s42255-019-0082-3 (2019).

37 Pereira, L., Yi, F. & Merrill, B. J. Repression of Nanog gene transcription by Tcf3 limits embryonic stem cell self-renewal. Molecular and cellular biology 26, 7479-7491, doi: 10.1128/MCB.00368-06 (2006).

38 Wray, J. et al. Inhibition of glycogen synthase kinase-3 alleviates Tcf3 repression of the pluripotency network and increases embryonic stem cell resistance to differentiation. Nat Cell Biol 13, 838-845, doi: 10.1038/ncb2267 (2011).

39 Moussaieff, A. et al. Glycolysis-mediated changes in acetyl-CoA and histone acetylation control the early differentiation of embryonic stem cells. Cell metabolism 21, 392-402, doi: 10.1016/j.cmet.2015.02.002 (2015).

40 Sullivan, L. B. et al. Supporting Aspartate Biosynthesis Is an Essential Function of Respiration in Proliferating Cells. Cell 162, 552-563, doi: 10.1016/j.cell.2015.07.017 (2015).

41 Borst, P. The malate-aspartate shuttle (Borst cycle): How it started and developed into a major metabolic pathway. IUBMB Life 72, 2241-2259, doi: 10.1002/iub.2367 (2020).

42 Assmann, N. et al. Srebp-controlled glucose metabolism is essential for NK cell functional responses. Nat Immunol 18, 1197-1206, doi: 10.1038/ni.3838 (2017).

43 Brunengraber, H. & Lowenstein, J. M. Effect of (−)-hydroxycitrate on ethanol metabolism. FEBS Lett 36, 130-132, doi: 10.1016/0014-5793 (73) 80353-9 (1973).

44 Luengo, A. et al. Increased demand for NAD(+) relative to ATP drives aerobic glycolysis. Molecular cell, doi: 10.1016/j.molcel.2020.12.012 (2020).

45 Zhu, X. G. et al. Functional Genomics In Vivo Reveal Metabolic Dependencies of Pancreatic Cancer Cells. Cell metabolism 33, 211-221 e216, doi: 10.1016/j.cmet.2020.10.017 (2021).

46 Dempster, J. M. et al. Extracting Biological Insights from the Project Achilles Genome-Scale CRISPR Screens in Cancer Cell Lines. bioRxiv, 720243, doi: 10.1101/720243 (2019).

47 Meyers, R. M. et al. Computational correction of copy number effect improves specificity of CRISPR-Cas9 essentiality screens in cancer cells. Nature genetics 49, 1779-1784, doi: 10.1038/ng.3984 (2017).

48 Ashburner, M. et al. Gene ontology: tool for the unification of biology. The Gene Ontology Consortium. Nature genetics 25, 25-29, doi: 10.1038/75556 (2000).

49 The Gene Ontology, C. The Gene Ontology Resource: 20 years and still Going strong. Nucleic Acids Res 47, D330-D338, doi: 10.1093/nar/gky 1055 (2019).

50 Virtanen, P. et al. SciPy 1.0: fundamental algorithms for scientific computing in Python. Nat Methods 17, 261-272, doi: 10.1038/s41592-019-0686-2 (2020).

51 Ghandi, M. et al. Next-generation characterization of the Cancer Cell Line Encyclopedia. Nature 569, 503-508, doi: 10.1038/s41586-019-1186-3 (2019).

52 Subramanian, A. et al. Gene set enrichment analysis: a knowledge-based approach for interpreting genome-wide expression profiles. Proceedings of the National Academy of Sciences of the United States of America 102, 15545-15550, doi: 10.1073/pnas.0506580102 (2005).

53 Dow, L. E. et al. Inducible in vivo genome editing with CRISPR-Cas9. Nat Biotechnol 33, 390-394, doi: 10.1038/nbt.3155 (2015).

54 Ran, F. A. et al. Genome engineering using the CRISPR-Cas9 system. Nature protocols 8, 2281-2308, doi: 10.1038/nprot.2013.143 (2013).

55 MacDougall, M. S., Clarke, R. & Merrill, B. J. Intracellular Ca(2+) Homeostasis and Nuclear Export Mediate Exit from Naive Pluripotency. Cell stem cell 25, 210-224 e216, doi: 10.1016/j.stem.2019.04.015 (2019).

56 Morris, J. P. t. et al. alpha-Ketoglutarate links p53 to cell fate during tumour suppression. Nature, doi: 10.1038/s41586-019-1577-5 (2019).

57 Fellmann, C. et al. An optimized microRNA backbone for effective single-copy RNAi. Cell Rep 5, 1704-1713, doi: 10.1016/j.celrep.2013.11.020 (2013).

58 Millard, P. et al. IsoCor: isotope correction for high-resolution MS labeling experiments. Bioinformatics 35, 4484-4487, doi: 10.1093/bioinformatics/btz209 (2019).

59 Chen, S., Zhou, Y., Chen, Y. & Gu, J. fastp: an ultra-fast all-in-one FASTQ preprocessor. Bioinformatics 34, i884-1890, doi: 10.1093/bioinformatics/bty560 (2018).

60 Dobin, A. et al. STAR: ultrafast universal RNA-seq aligner. Bioinformatics 29, 15-21, doi: 10.1093/bioinformatics/bts635 (2013).

61 Liao, Y., Smyth, G. K. & Shi, W. featureCounts: an efficient general purpose program for assigning sequence reads to genomic features. Bioinformatics 30, 923-930, doi: 10.1093/bioinformatics/btt656 (2014).

62 Love, M. I., Huber, W. & Anders, S. Moderated estimation of fold change and dispersion for RNA-seq data with DESeq2. Genome biology 15, 550, doi: 10.1186/s13059-014-0550-8 (2014).

1.-48. (canceled)

49. A method for maintaining pluripotency of cells, comprising blocking non-canonical tricarboxylic acid (TCA) cycle of the cells, wherein the blocking comprises contacting the cells with an ATP citrate lyase (ACL) inhibitor.

50. The method of claim 49, wherein the ACL inhibitor is a synthetic ACL inhibitor, a natural ACL inhibitor, or a combination thereof.

51. The method of claim 49, wherein the concentration of the ACL inhibitor is between about 10 μM and about 100 μM.

52. The method of claim 49, wherein the cells are contacted with the ACL inhibitor for at least about 12 hours.

53. The method of claim 49, wherein the cells are contacted with the ACL inhibitor for about 24 hours.

54. A method for maintaining self-renewal property of cells, comprising blocking non-canonical tricarboxylic acid (TCA) cycle of the cells, wherein the blocking comprises contacting the cells with an ATP citrate lyase (ACL) inhibitor.

55. The method of claim 54, wherein the ACL inhibitor is a synthetic ACL inhibitor, a natural ACL inhibitor, or a combination thereof.

56. The method of claim 54, wherein the concentration of the ACL inhibitor is between about 10 μM and about 100 μM.

57. The method of claim 54, wherein the cells are contacted with the ACL inhibitor for at least about 12 hours.

58. The method of claim 54, wherein the cells are contacted with the ACL inhibitor for about 24 hours.

59. A plurality of cells, wherein pluripotency of the cells is maintained, after blocking the non-canonical tricarboxylic acid (TCA) cycle of the cells, wherein blocking the non-canonical TCA cycle comprises contacting the cells with an ATP citrate lyase (ACL) inhibitor.

60. The plurality of cells of claim 59, wherein the ACL inhibitor is a synthetic ACL inhibitor, a natural ACL inhibitor, or a combination thereof.

61. The plurality of cells of claim 59, wherein the concentration of the ACL inhibitor is between about 10 μM and about 100 μM.

62. The plurality of cells of claim 59, wherein the cells were contacted with the ACL inhibitor for at least about 12 hours.

63. he plurality of cells of claim 59, wherein the cells were contacted with the ACL inhibitor for about 24 hours.

64. A plurality of cells, wherein self-renewal property of the cells is maintained, after blocking the non-canonical tricarboxylic acid (TCA) cycle of the cells, wherein blocking the non-canonical TCA cycle comprises contacting the cells with an ATP citrate lyase (ACL) inhibitor.

65. The plurality of cells of claim 64, wherein the ACL inhibitor is a synthetic ACL inhibitor, a natural ACL inhibitor, or a combination thereof.

66. The plurality of cells of claim 64, wherein the concentration of the ACL inhibitor is between about 10 μM and about 100 μM.

67. The plurality of cells of claim 64, wherein the cells were contacted with the ACL inhibitor for at least about 12 hours.

68. The plurality of cells of claim 64, wherein the cells were contacted with the ACL inhibitor for about 24 hours.
